(19) United States
(12) Patent Application Publication
(10) Pub. No.: US 2022/0135971 A1
(43) Pub. Date: May 5, 2022

(54) CORE MASTER REGULATORS OF GLIOBLASTOMA STEM CELLS

(71) Applicant: UNIVERSITY OF FLORIDA RESEARCH FOUNDATION, INCORPORATED, Gainesville, FL (US)

(72) Inventors: David Tran, Gainesville, FL (US); Son Bang Le, Gainesville, FL (US)

(21) Appl. No.: 17/428,722

(22) PCT Filed: Feb. 6, 2020
(86) PCT No.: PCT/US20/17090

§ 371 Date: Aug. 5, 2021

Related U.S. Application Data
(60) Provisional application No. 62/802,554, filed on Feb. 7, 2019.

(51) Int. Cl. C12N 15/113; C12N 15/86; A61P 35/00; A61K 31/7088

(52) U.S. Cl. CPC C12N 15/113; C12N 15/86; A61P 35/00; A61K 31/7088; C12N 2320/32; C12N 2310/14; C12N 2310/122; C12N 2310/531; C12N 2740/15043

(57) ABSTRACT

Provided methods of inhibiting a glioblastoma stem-like cell (GSC), methods of treating a subject with glioblastoma, and methods of reprogramming an astrocyte to a glioblastoma stem-like cell (GSC).

### FEDERALLY SPONSORED RESEARCH OR DEVELOPMENT

This invention was made with government support K08CA160824 awarded by National Institute of Health (NIH). The government has certain rights in the invention.

### BACKGROUND

GBMs are heterogeneous tumors that arise from astrocytes—the star-shaped cells that make up the “glue-like” or supportive tissue of the brain. Glioblastomas usually contain a mix of cell types. It is not unusual for these tumors to contain cystic mineral, calcium deposits, blood vessels, or a mixed grade of cells, and are nourished by an ample blood supply. Recent advances in treatment for patients with glioblastoma (GBM) have produced only a modest survival benefit with few long-term survivors. New effective and safe therapies are urgently needed to enhance outcomes for GBM patients.

### BRIEF SUMMARY

Provided are compositions and methods for treating cancer. In one aspect, the cancer is a glioblastoma (GBM).

In one embodiment, a method of reprogramming an astrocyte to a glioblastoma stem-like cell (GSC) by introducing at least one master regulator selected from the group consisting of: NKX2-2, ETV4, MLXIPL, MEOX2, PRKCB, DDN, or OTP into a cell.

In another embodiment, a method of inhibiting a glioblastoma stem-like cell (GSC) by administering an immunotherapy composition that inhibits or reduces the expression of at least one master regulator selected from the group consisting of: NKX2-2, ETV4, MLXIPL, MEOX2, PRKCB, DDN, or OTP.

In another aspect, a method of treating a subject for glioblastoma by administering an immunotherapy composition that inhibits or reduces the expression of at least one master regulator selected from the group consisting of: NKX2-2, ETV4, MLXIPL, MEOX2, PRKCB, DDN, or OTP.

In another embodiment, an immunotherapy composition for treating a subject with a glioblastoma, comprising an inhibitor of at least one of NKX2-2, ETV4, MLXIPL, MEOX2, PRKCB, DDN, or OTP.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 illustrates master regulators of GSCs identified using GeneRep-nSCORE.

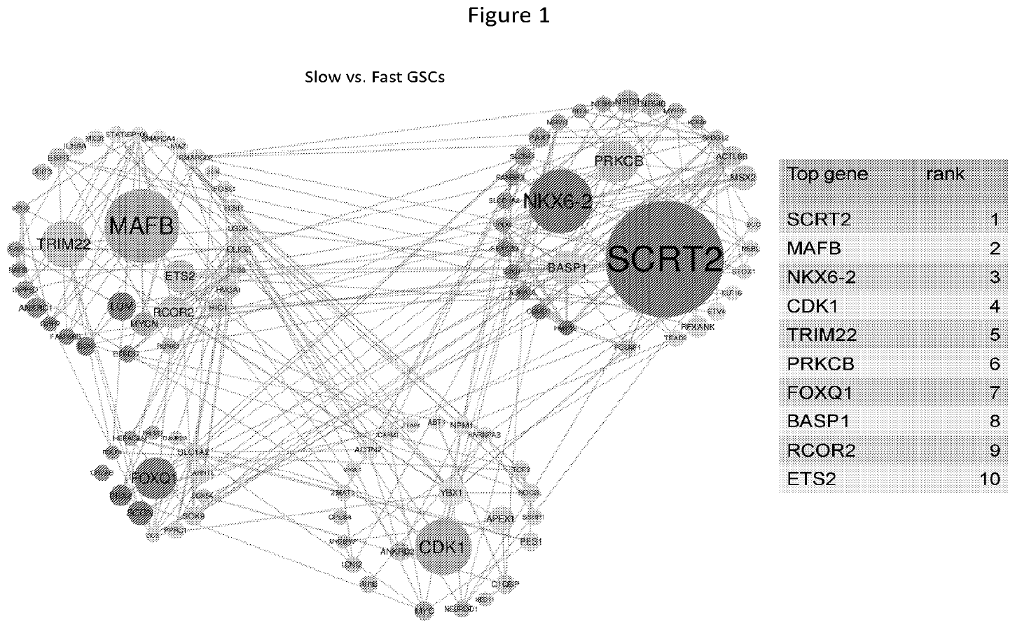

FIG. 2 illustrates that NKX6-2 is preferentially expressed in slow cycling GSCs compared to fast cycling GSCs. ASCL1 is expressed in both GSC populations. Using fast-cycling and slow-cycling GSCs as model we explored the function of ASCL1 & NKX6.2 in GSCs proliferation and survival.

FIG. 3 illustrates NKX6.2 is essential for slow cycling, but not fast cycling GSCs.

FIG. 4 illustrates partial reprogramming of astrocytes to GSCs with ASCL1, BASP1, MYCN, SOX8 (ABMNS).

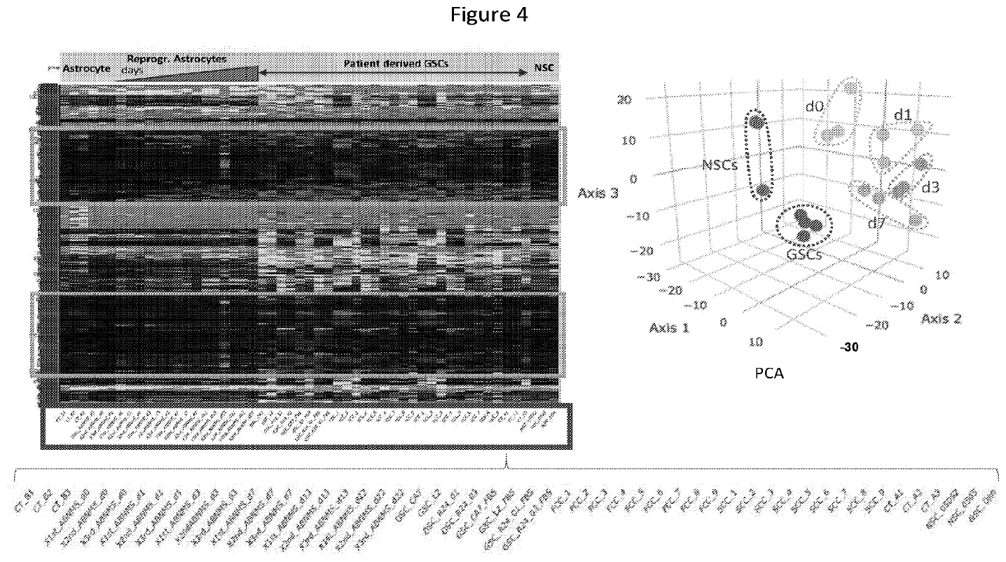

FIG. 5 illustrates master regulators to reprogram astrocytes to GSCs.

FIG. 6 illustrates expression of master regulators in GSCs.

FIG. 7 illustrates knockdown of master regulators leads to GSC death.

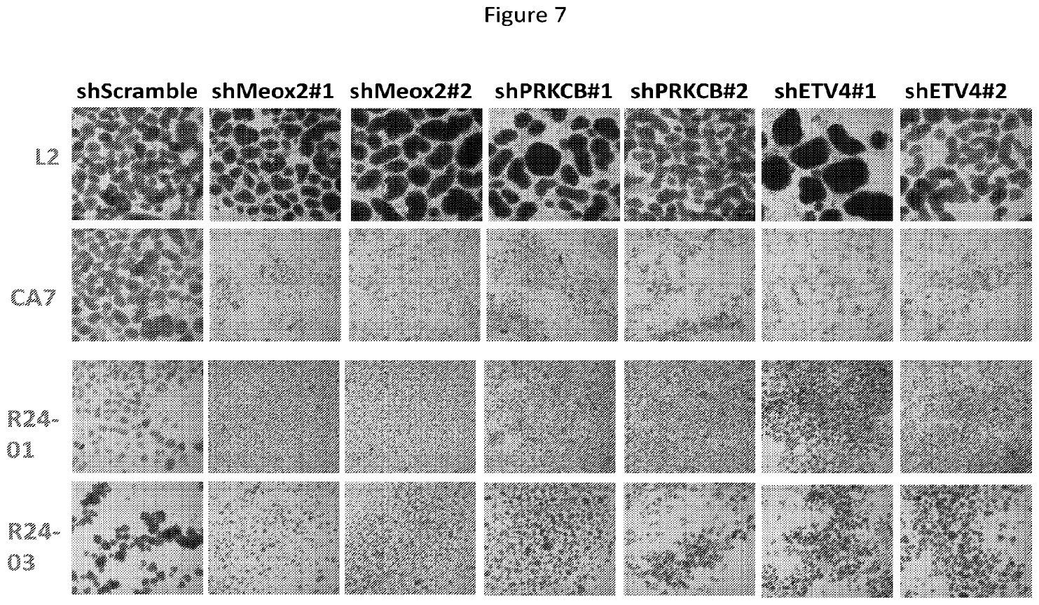

FIG. 8 illustrates knockdown of master regulators leads to GSC death.

FIG. 9 illustrates knockdown of master regulators leads to GSC death.

FIG. 10 illustrates double knockdown of master regulators leads to GSC death.

FIG. 11 illustrates single and double knockdown of master regulators leads to GSC death.

FIG. 12 illustrates survival curves in mice administered GSC cells with partial knockdown of mater regulators.

### DEFINITIONS

A “master regulator” or “cancer master regulator” is a gene or protein that acts to drive one or more intermediary gene or proteins in a pathway or network important in initiating or maintaining a cancerous state or initiating or maintaining one or more deleterious cancerous behaviors. Some master regulators are involved in pathways in the transition to a cancer state.

A “master regulator network” refers to a master regulator and one or more genes downstream of the master regulator whose transcription level is dependent on or affected by the master regulator.

The terms “protein,” “polypeptide,” and “peptide,” used interchangeably herein, refer to polymeric forms of amino acids of any length, including coded and non-coded amino acids and chemically or biochemically modified or derivatized amino acids. The terms include polymers that have been modified, such as polypeptides having modified peptide backbones.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” refers to a process of modifying a nucleic acid sequence for enhanced expression in particular host cells by replacing at least one codon of the native sequence with a codon that is more frequently or most frequently used in the genes of the host cell while maintaining the native amino acid sequence. For example, a polynucleotide encoding a fusion polypeptide can be modified to substitute codons having a higher frequency of usage in a given host cell as compared to the naturally occurring nucleic acid sequence. Codon usage tables are readily available, for example, at the “Codon Usage Database.” The optimal codons utilized by L. monocytogenes for each amino acid are shown US 2007/0207170, herein incorporated by reference in its entirety for all purposes. These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Asp artic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “fragment” when referring to a protein means a protein that is shorter or has fewer amino acids than the full length protein. The term “fragment” when referring to a nucleic acid means a nucleic acid that is shorter or has fewer nucleotides than the full length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### DETAILED DESCRIPTION

Various embodiments of the inventions now will be described more fully hereinafter, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level.

Glioblastoma (GBM) is the most common and lethal form of adult human brain cancers. GBMs are formed by GBM stem-like cells (GSCs)—a major contributor to tumor recurrence and a natural focus for therapeutic development. There are two main reasons responsible for treatment failure: 1) high cellular and molecular heterogeneity; 2) GSCs have multiple redundant pathways requiring simultaneous targeting.

Details regarding various embodiments are described herein. By way of background, GBM is enriched in GBM stem-like cells (GSCs), a major contributor to tumor recurrence. Both GSCs and normal neuronal precursor cells (NPC) have the ability to form neurospheres when cultured in stem cell conditions. However, only GSCs can regenerate all cancer cells in the tumor when implanted in vivo (e.g., in vivo tumorigenicity). GSCs also can differentiate into other cells of the brain, however these cells are often not functional compared to those produced by NPCs. In a mouse model of GBM, elimination of self-renewal by genetic means led to a loss of GSCs and prolonged survival. However, as with other cancers, targeting GSCs has been a challenge because of the dearth of master regulators specific only to GSCs and not to NPCs or normal brain cells. The cell origin of GSCs remains unclear; both NPCs and normal astrocytes (NA) have been shown to contribute to GSCs. As a result, several survival and growth signals in GSCs share parallels in NPCs and NAs, increasing potential toxicity for therapies that target these pathways. Many of these targets are downstream signaling nodes with overlapping functions, allowing them to compensate for one another's blockade. Another challenge is the high intra- and inter-tumor heterogeneity in the GSC compartment, which necessitates the development of therapies that can target most, if not all, fractions of different subclones within and across many tumors. Recent genomics studies suggest that like other cancers, GBM originates from a founding GSC clone that emerged after sustaining a series of initiating and cooperative alterations that are passed on such that all subclones contain the founding alterations (i.e., the core common master regulators) and hence are targetable. As the number of potential founding alterations is surprisingly small, many founding alterations are expected to be common across different tumors of the same type or even of different types.

Founding alterations may produce “imprints” on the global gene regulatory network that may persist as the founding clone morphs into subclones and may be traceable across subclones. However, understanding the biological implications of these genomic alterations requires novel analytic tools that interrogate large-scale gene expression profiles to provide information on cancer cell's behaviors caused by interactions between the founding alterations and the tumor microenvironment. Gene expression profiles can then be used to infer the global and local networks that control such behaviors. This can be achieved using reverse engineering tools such as ARACNe (Algorithm for the Reconstruction of Accurate Cellular Networks), designed to scale up to the complexity of mammalian cells. ARACNe applies a theoretical information approach to infer gene networks using gene expression data, by calculating Mutual Information (MI).

In some embodiments, two computational engines GeneRep and nSCORE are applied to optimize the use of ARACNe and to quantitatively rank master regulators in any network, respectively. This strategy is greatly enhanced by the coupling with a multi-pronged compound-screening scheme.

### Identification of Master Regulators of Gene Networks

GeneRep and nSCORE address two difficulties in computational biology: how to set a threshold cutoff level to maximize sensitivity while minimizing the false discovery rate (FDR) and how to incorporate various ranking parameters known individually to influence network hierarchy. GeneRep employs innovative coupling of bootstrapping with a random networks generation procedure from the real data. Networks generated at the gene level by GeneRep contain 20,000 nodes, while those generated at the transcript level contain 50,000 nodes. The number of edges ranges from 300,000 to 1 million, far higher than what is often obtained with current methods. nSCORE creates an automated node importance scoring framework that incorporates limitless sets of existing parameters and thus can be applied to any type of networks and node statistics inputs. GeneRep-nSCORE is described in WO-2018/069891, which is incorporated by reference in its entirety.

The master regulator identification and targeting workflow integrates key aspects to optimize success: GeneRep-nSCORE to rapidly identity GSC-specific master regulators at apices of signaling networks; intra- and inter-tumor heterogeneity analyses to identify master regulators common among GSC subclones; mutational and survival analyses to capture additional relevant master regulators; a two-pronged compound screening platform combining in silico and ultra-high throughput functional screens; evaluation of the clinical timeframe from surgery to drug identification; and development of a quantitative, network-based predictive biomarker for treatment response in GSCs.

We previously elucidated the roles of BASP1, NKX6.2, STOX2, MYCN, SOX8, OLIG2, HES6, and ASCL1 in reprogramming AST to GSC in WO 2018/211409, which is incorporated herein in its entirety.

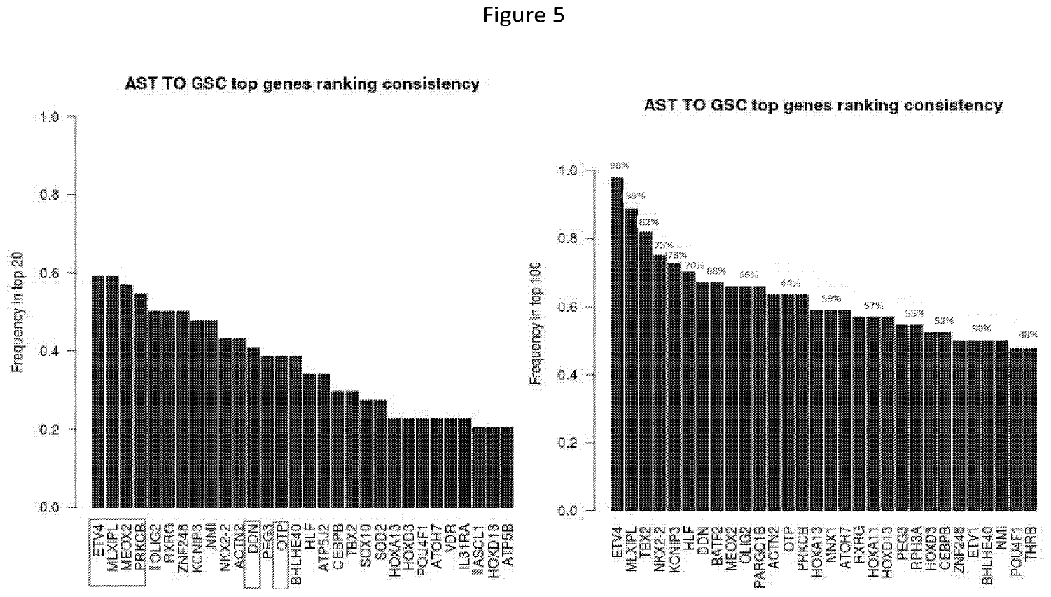

Here we disclose further genes that play a role in reprogramming AST to GSC. FIG. 5 shows top ranking genes:ETV4, MLXIPL, MEOX2, PRKCB, OLIG2, RXRG, ZNF248, KCNIP3, NMI, NKX2-2, ACTN2, DDN, PEG3, OTP, BHLHE40, HLF, ATP5J2, CEBPB, TBX2, SOX10, SOD2, HOXA13, HOXD3, POU4F1, ATOH7, VDR, IL31RA, ASCL1, HOXD13, ATP5B, BATF2, PARGC1B, HOXA11, RPH3A, ETV1, THRB, and MNX1.

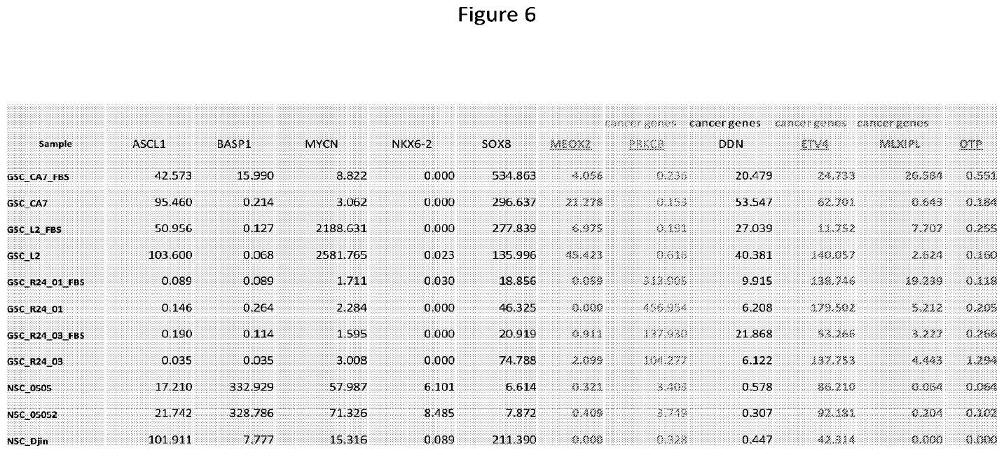

Additionally, we take a closer look at a subset of master regulators involved in reprograming astrocytes to GSCs, i.e., MEOX2, PRKCB, DDN, ETV4, MLXIPL, and OTP in combination with ASCL1, BASP1, MYCN, NKX6-2, and SOX8 (FIG. 6). These master regulators were selected either because they have the largest fold change between GSCs and GBM differentiating cells (GDCs) or because they have the highest frequency occurring in top ranked genes across multiple samples and patients.

NKX2-2 (NK2 Homeobox 2) encodes a protein that contains a homeobox domain and may be involved in the morphogenesis of the central nervous system. Diseases associated with NKX2-2 include Maturity-Onset Diabetes Of The Young and Cranial Nerve Malignant Neoplasm. Among its related pathways are Developmental Biology and Embryonic and Induced Pluripotent Stem Cell Differentiation Pathways and Lineage-specific Markers.

MEOX2 (Mesenchyme Homeobox 2) is a protein coding gene. Diseases associated with MEOX2 include Female Stress Incontinence and Low Compliance Bladder. Gene Ontology (GO) annotations related to this gene include DNA-binding transcription factor activity and RNA polymerase II proximal promoter sequence-specific DNA binding.

PRKCB (Protein Kinase C Beta) is a member of the protein kinase C (PKC) family of serine- and threonine-specific protein kinases that can be activated by calcium and second messenger diacylglycerol. PKC family members phosphorylate a wide variety of protein targets and are known to be involved in diverse cellular signaling pathways. PKC family members also serve as major receptors for phorbol esters, a class of tumor promoters. Each member of the PKC family has a specific expression profile and is believed to play a distinct role in cells. The protein encoded by this gene is one of the PKC family members. This protein kinase has been reported to be involved in many different cellular functions, such as B cell activation, apoptosis induction, endothelial cell proliferation, and intestinal sugar absorption. Studies in mice also suggest that this kinase may also regulate neuronal functions and correlate fear-induced conflict behavior after stress.

DDN (Dendrin) is a protein coding gene. The DDN protein has been associated with promoting apoptosis of kidney glomerular podocytes.

ETV4 (ETS Variant Transcription Factor 4) is a protein coding gene. Diseases associated with ETV4 include Ewing Sarcoma and Extraosseous Ewing Sarcoma. Among its related pathways are RET signaling and Transcriptional misregulation in cancer.

MLXIPL (MLX Interacting Protein Like) encodes a basic helix-loop-helix leucine zipper transcription factor of the Myc/Max/Mad superfamily. This protein forms a heterodimeric complex and binds and activates, in a glucose-dependent manner, carbohydrate response element (ChoRE) motifs in the promoters of triglyceride synthesis genes. The gene is deleted in Williams-Beuren syndrome, a multisystem developmental disorder caused by the deletion of contiguous genes at chromosome 7q11.23.

OTP (Orthopedia Homeobox) encodes a member of the homeodomain (HD) family. HD family proteins are helix-turn-helix transcription factors that play key roles in the specification of cell fates.

In embodiments, a method of reprogramming an astrocyte to a glioblastoma stem-like cell (GSC) by introducing at least one master regulator selected from the group consisting of: NKX2-2, ETV4, MLXIPL, MEOX2, PRKCB, DDN, or OTP into a cell. In embodiments, a method of reprogramming an astrocyte to a glioblastoma stem-like cell (GSC) by introducing at least one master regulator selected from the group consisting of: NKX2-2, ETV4, MLXIPL, MEOX2, PRKCB, DDN, or OTP and further comprising introducing at least one master regulator from the group consisting of: BASP1, NKX6.2, STOX2, MYCN, SOX8, OLIG2, HES6, and ASCL1 into a cell.

In embodiments, a method of reprogramming an astrocyte to a glioblastoma stem-like cell (GSC) by introducing at least two master regulators selected from the group consisting of: NKX2-2, ETV4, MLXIPL, MEOX2, PRKCB, DDN, or OTP and further comprising introducing at least one master regulator from the group consisting of: BASP1, NKX6.2, STOX2, MYCN, SOX8, OLIG2, HES6, and ASCL1 into a cell. In embodiments, a method of reprogramming an astrocyte to a glioblastoma stem-like cell (GSC) by introducing at least 3, 4, 5, 6, or 7 master regulators selected from the group consisting of: NKX2-2, ETV4, MLXIPL, MEOX2, PRKCB, DDN, or OTP and further comprising introducing at least one master regulator from the group consisting of: BASP1, NKX6.2, STOX2, MYCN, SOX8, OLIG2, HES6, and ASCL1 into a cell.

In embodiments, a method of reprogramming an astrocyte to a glioblastoma stem-like cell (GSC) by introducing at least one master regulator selected from the group consisting of: NKX2-2, ETV4, MLXIPL, MEOX2, PRKCB, DDN, or OTP and further comprising introducing at least two master regulators from the group consisting of: BASP1, NKX6.2, STOX2, MYCN, SOX8, OLIG2, HES6, and ASCL1 into a cell. In embodiments, a method of reprogramming an astrocyte to a glioblastoma stem-like cell (GSC) by introducing at least one master regulator selected from the group consisting of: NKX2-2, ETV4, MLXIPL, MEOX2, PRKCB, DDN, or OTP and further comprising introducing at least 3, 4, 5, 6, 7, or 8 master regulators from the group consisting of: BASP1, NKX6.2, STOX2, MYCN, SOX8, OLIG2, HES6, and ASCL1 into a cell.

In embodiments, a method of reprogramming an astrocyte to a glioblastoma stem-like cell (GSC) by introducing NKX2-2, ETV4, MLXIPL, MEOX2, PRKCB, DDN, or OTP and further comprising introducing BASP1, NKX6.2, STOX2, MYCN, SOX8, OLIG2, HES6, and ASCL1 into a cell.

### Methods of Treatment

The presently disclosed subject matter provides master regulators, such as NKX2-2, MEOX2, PRKCB, DDN, ETV4, MLXIPL, and OTP that when inhibited, can reduce or inhibit GSCs. In some embodiments, inhibition of at least one of these master regulators can be used to inhibit GSCs. In some embodiments, inhibition of a combination of at least two of these master regulators can be used to inhibit GSCs. In some embodiments, inhibition of at least one of these master regulators can be used to treat a subject with glioblastoma. In some embodiments, a combination of inhibition of at least two of these master regulators can be used to treat a subject with glioblastoma. In some embodiments, the presently disclosed subject matter provides a method of reprogramming normal human astrocytes to GSCs by introducing a combination of the master regulators disclosed herein into a cell. In some embodiments, inhibition of a combination of the master regulators NKX2-2, MEOX2, PRKCB, DDN, ETV4, MLXIPL, and OTP can be used to inhibit GSCs or in therapeutic methods for treating glioblastoma.

In some embodiments, a method of inhibiting GSCs or treating glioblastoma comprising using or administering an immunotherapy composition against individual or combinations of the master regulators disclosed herein. Also provided are immunotherapy compositions that target at least one of the master regulators disclosed herein In one embodiment, the immunotherapy composition comprises a peptide formulation derived from at least one of the master regulators disclosed herein. In one embodiment, the immunotherapy composition comprises nanoparticle or dendritic cell containing peptides derived from at least one of the master regulators disclosed herein. In one embodiment, the immunotherapy composition comprises RNAs coding for at least one of the master regulators disclosed herein. In one embodiment, the immunotherapy composition comprises nanoparticles or dendritic cells containing RNAs coding for at least one master regulator disclosed herein. In one embodiment, the RNAs coding for master regulators are electroporated into dendritic cells.

Also provided are pharmaceutical compositions that inhibit at least one master regulator disclosed herein. In one embodiment, the inhibitor is a RNA interference agent or a small molecule.

In one embodiment, delivery of the composition is by direct injection into the brain. In one embodiment, delivery is by gene therapy, for example by adeno-associated virus (AAV) or retroviral replication vector (RRV) vector. In one embodiment, delivery is by systemic intravenous delivery.

In some embodiments, we describe methods of treating cancer comprising inhibiting one or more master regulators. Inhibiting one or more master regulators can comprise using or administering one or more master regulator antagonists or inhibitors. A master regulator can be inhibited at the gene level, such as by using or administering RNA interference agents or antisense oligonucleotides to inhibit expression of the gene. The master regulators can be inhibited at the protein level, such as by using or administering an immunotherapy composition that binds to the master regulator protein and inhibits activity of the protein or by using or administering a small molecule drug known to inhibit activity of the master regulator protein. In some embodiments, we described methods of treating cancer comprising using or administering an immunotherapy composition against a master regulator protein or a combination of master regulator proteins. An immunotherapy composition can comprise one or more antibodies having affinity for one or more master regulators. An antibody can be, but is not limited to, an immunoglobulin, an immunoglobulin fragment having affinity for the master regulator, a chimeric antibody, a bispecific antibody, an antibody conjugate, or the like.

In some embodiments, an immunotherapy composition comprises a peptide formulation derived from a master regulator. The peptide can be an immunogenic fragment of a master regulator protein. The peptide can be combined with an immune stimulating adjuvant. The immunotherapy composition can be administered locally (e.g., subcutaneously) or systemically (e.g., intravenously) with or without the presence of adjuvant. The immunotherapy composition can be used to stimulate the immune system to develop an immune reaction specifically against the master regulator. Development of an immune reaction can eliminate or aid in eliminating cancer cells expressing the master regulator.

In some embodiments, we describe methods of treating cancer comprising using or administering one or more small molecule drugs to inhibit activity of a master regulator protein or a combination of master regulator proteins. In embodiments, the method comprises administering immunotherapy compositions, small molecules, RNA interference agents, antisense oligonucleotides, or combinations thereof that target one or more of the master regulators associated with the cancer.

In some embodiments, we describe methods of treating cancer comprising using or administering one or more antisense oligonucleotides or RNA interference agents to knock down expression of a master regulator gene or a combination of master regulator genes. An antisense oligonucleotide is a single-stranded oligonucleotide having a nucleobase sequence that permits hybridization to a corresponding region or segment of a target nucleic acid. An RNA interference agent is an oligonucleotide that mediates the targeted cleavage of an RNA transcript in a sequence specific manner via an RNA-induced silencing complex (RISC) pathway.

In some embodiments, we describe methods of treating cancer comprising using or administering a combination of one or more master regulator antagonists or inhibitors.

In one embodiment, the master regulator is NKX2-2. In one embodiment, NKX2-2 has the sequence of SEQ ID No: 2 or NG 042186.1. In one embodiment, a method of treating a cancer or tumor by administering and inhibitor of NKX2-2 to a subject in need thereof. In one embodiment, the inhibitor that targets NKX2-2 targets SEQ ID No: 2 or NG 042186.1 or a fragment thereof.

In one embodiment, the master regulator is MLXIPL. In one embodiment, MLXIPL has the sequence of SEQ ID Nos: 4, 6, 8, 10, or NG 009307.1. In one embodiment, a method of treating a cancer or tumor by administering and inhibitor of MLXIPL to a subject in need thereof. In one embodiment, the inhibitor that targets MLXIPL targets SEQ ID Nos: 4, 6, 8, 10, NG 009307.1, or a fragment thereof.

In one embodiment, the master regulator is ETV4. In one embodiment, ETV4 has the sequence of SEQ ID No: 12, 14, 16, 18, 20, or NC_000017.11. In one embodiment, a method of treating a cancer or tumor by administering and inhibitor of ETV4 to a subject in need thereof. In one embodiment, the inhibitor that targets ETV4 targets SEQ ID No: 12, 14, 16, 18, 20, NC_000017.11, or a fragment thereof.

In one embodiment, the master regulator is MEOX2. In one embodiment, MEOX2 has the sequence of SEQ ID No: 22 or NG_032988.1. In one embodiment, a method of treating a cancer or tumor by administering and inhibitor of MEOX2 to a subject in need thereof. In one embodiment, the inhibitor that targets MEOX2 targets SEQ ID No: 22 or NG_032988.1 or a fragment thereof.

In one embodiment, the master regulator is PRKCB. In one embodiment, PRKCB has the sequence of SEQ ID No: 24 or 26 or NG_029003.2. In one embodiment, a method of treating a cancer or tumor by administering and inhibitor of PRKCB to a subject in need thereof. In one embodiment, the inhibitor that targets PRKCB targets SEQ ID No: 24 or 26 or NG_029003.2 or a fragment thereof.

In one embodiment, the master regulator is DDN. In one embodiment, DDN has the sequence of SEQ ID No: 28 or NC_000012.12. In one embodiment, a method of treating a cancer or tumor by administering and inhibitor of DDN to a subject in need thereof. In one embodiment, the inhibitor that targets DDN targets SEQ ID No: 28 or NC_000012.12 or a fragment thereof.

In one embodiment, the master regulator is OTP. In one embodiment, OTP has the sequence of SEQ ID No: 30 or NC_000005.10. In one embodiment, a method of treating a cancer or tumor by administering and inhibitor of OTP to a subject in need thereof. In one embodiment, the inhibitor that targets OTP targets SEQ ID No: 30 or NC_000005.10 or fragment thereof.

In one embodiment, a method of treating a subject with a cancer or tumor comprising administering a composition comprising an inhibitor of at least one master regulator disclosed herein. In one embodiment, the master regulator is selected from the group consisting of NKX2-2, MEOX2, PRKCB, DDN, ETV4, MLXIPL, and OTP.

In one embodiment, a method of treating a subject with a cancer or tumor. In one embodiment, the cancer or tumor is a glioblastoma. In one embodiment, the tumor is a glioma. In one embodiment, the tumor is from brain. In one embodiment, the cancer or tumor is non-small cell lung cancer or cancer where the cell type of origin are from neurodectoderm.

### EXAMPLES

### Example 1: Identification of Master Regulators for Slow-Cycling GSCs Versus Fast-Cycling GSCs

There are two different populations of GSCs, slow-cycling and fast-cycling. Slow-cycling GSCs are slow-dividing but they give rise to fast-cycling GSCs, which are fast-dividing. Fast-cycling GSC are more susceptible to therapeutics since they are target fast-dividing. Therefore, targeting the slow-cycling GSCs will destroy the tumor since slow-cycling GSCs replenish the fast-dividing GSCs which are dying off due to cancer therapeutics.

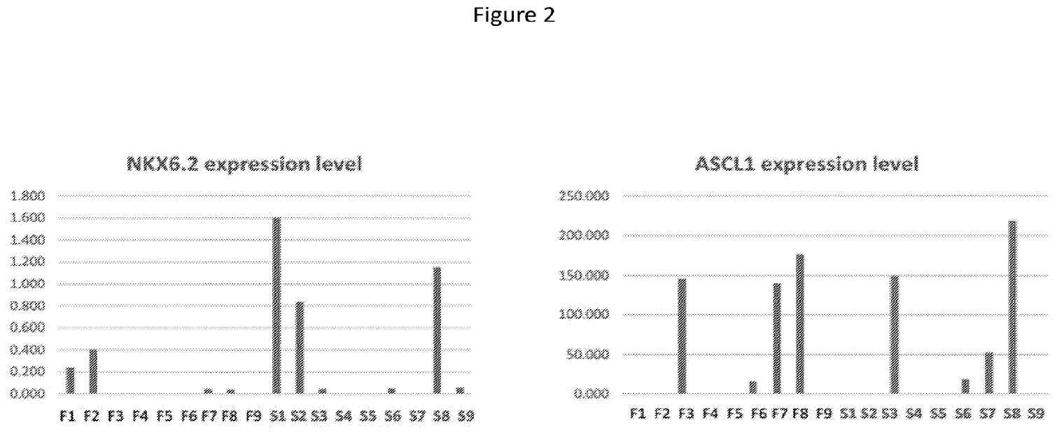

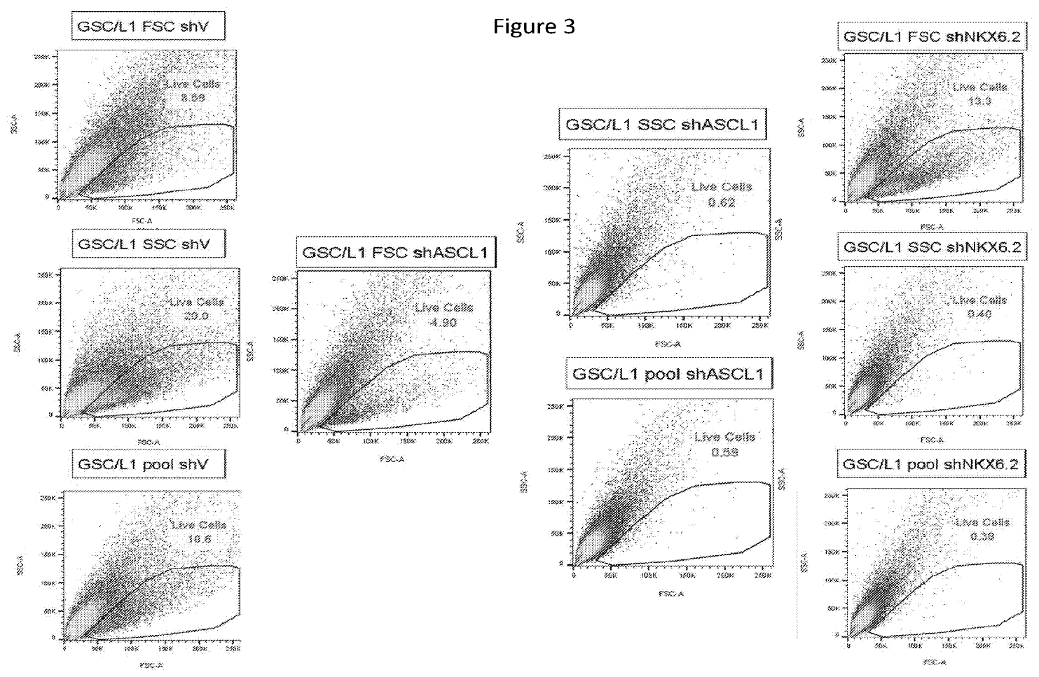

Here, we explore the GSCs master regulators NKX6.2 and ASCL1 and whether expression is specific to regulating slow-cycling GSCs or fast-cycling GSCs or both. We show that NKX6.2 preferentially expressed and is essential for slow cycling GSCs, but not fast cycling GSCs (FIGS. 2 and 3). Since slow-cycling GSCs give rise to fast-cycling GSCs and are necessary for tumor growth and maintenance, NKX6.2 is a promising target for treating GBM by specifically targeting slow-cycling GSCs. For example, inhibiting the expression of NKX6.2, e.g., either by genetic means (si/shRNA) or small molecule inhibitors, may have significant therapeutic potential as a treatment of GBM that specifically targets slow-cycling GSCs, and possibly for other cancers whose stem cells share similar regulatory pathways.

Master regulators are genes at the top of a gene network which can alter the expression of downstream genes in a network. Applying the tandem computational platform GeneRep-nSCORE that integrates large-scale gene expression profiles with genomic changes to identify common founding master regulators of GSCs spanning across most, if not all, GSC clones, we discovered set of common master regulators in GCSs that are outstanding targets for clinical development.

### Example 2: In Vitro Single and Double Knockdown Experiments

We applied the GeneRep-nSCORE platform to gene expression profiles of GSCs and GBM differentiating cells (GDC), normal neuronal precursor cells (NPC), and normal human astrocytes (NHA) and predicted top genes involved in fate conversions between these cell types.

Here, we take a closer look at a subset of the master regulators: MEOX2, PRKCB, ETV4, along with NKX6-2.

We used lentiviruses encoding for shRNA specific for one or two master regulators and transduced 3 independent patient-derived GSC lines. These results confirmed that effective inhibition of one or two master regulators, either by genetic means (si/shRNA) or perhaps small molecule inhibitors, would have significant therapeutic potential as a GSC-specific treatment of GBM, and possibly for other cancers whose stem cells share similar regulatory pathways.

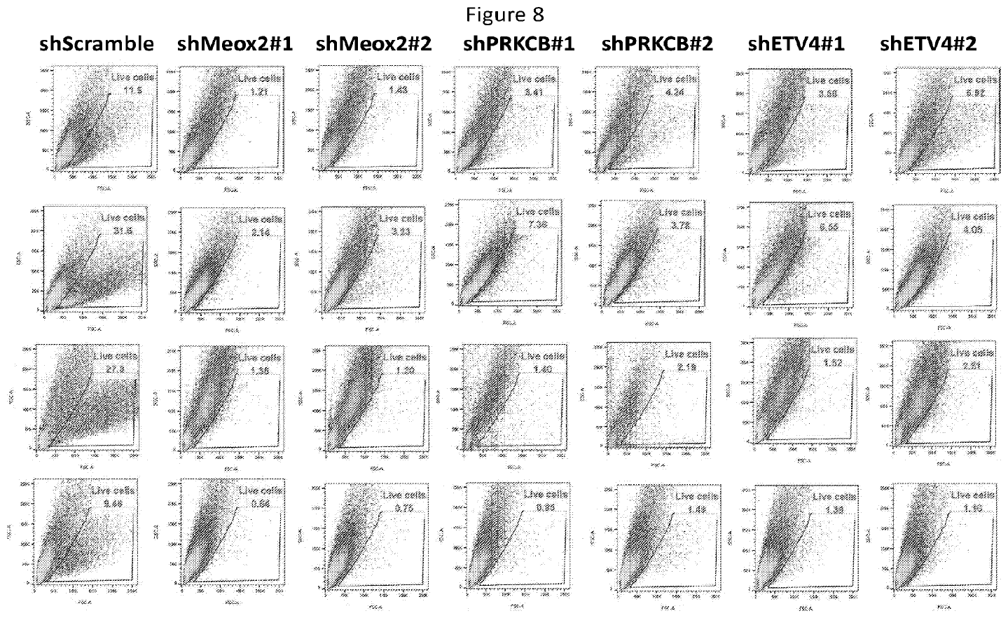

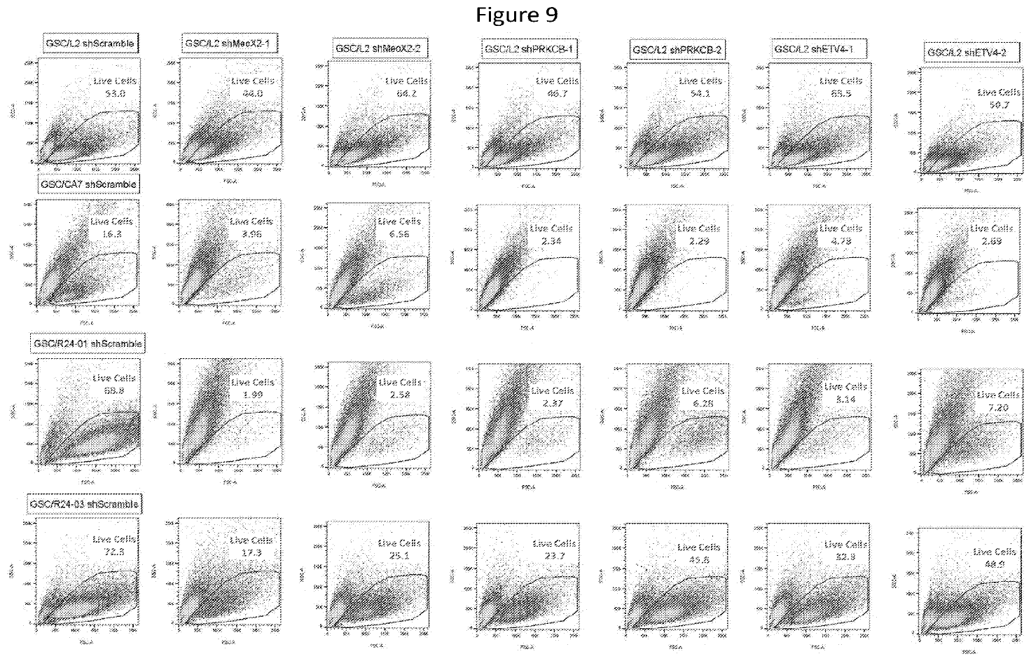

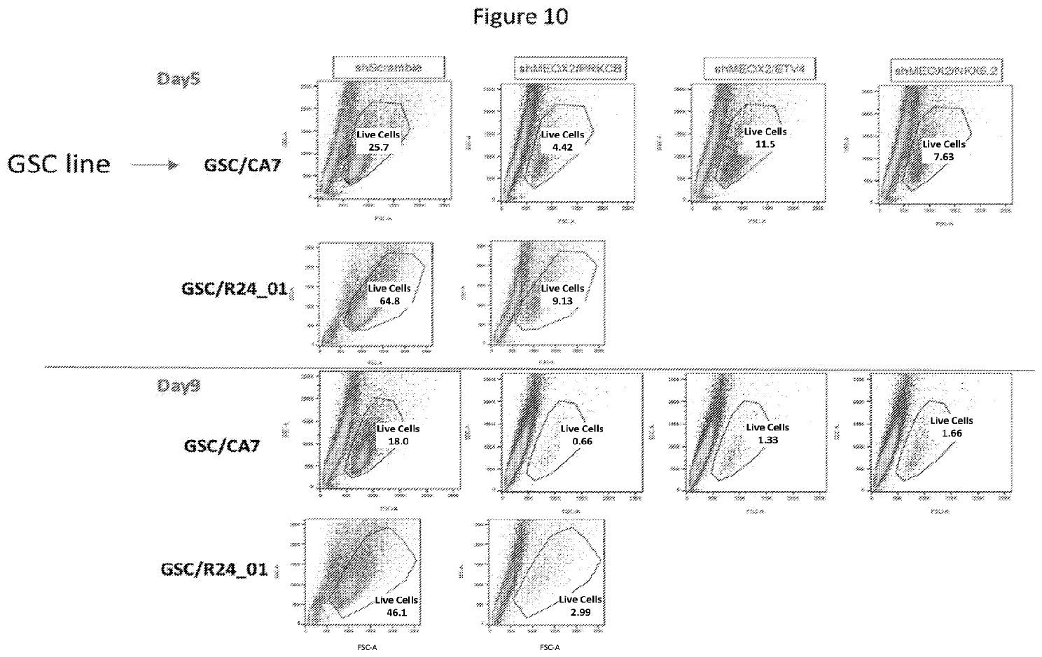

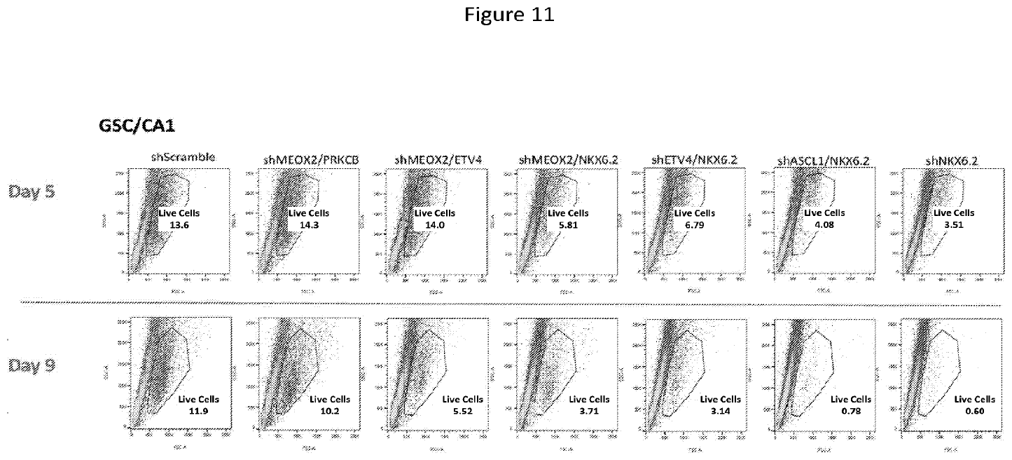

FIGS. 8 through 11 show the results of the in vitro knockdown experiments. FIGS. 8 and 9 shows that single knockdown of MEOX2, PRKCB, or ETV4 leads to GSC death. FIGS. 10 and 11 show that double knockdown of MEOX/PRKCB, MEOX/ETV4, or MEOX/NKX6-2 leads to GSC death.

These experiments were performed in 3 individual patient derived GSC cell lines (CA7, R24-03, or R24-01) and to the same result. Together, these findings show that these master regulators may serve as important pharmacologically targets that and may reduce tumorigenicity (i.e., reduced tumor size or number of tumors).

### Example 3: In Vivo Experiments in Mice

Combinations of MEOX2 and PRKCB, MEOX2 and ETV4, MEOX2 and NKX6.2, and ASCL1 and NKX6.2 were tested in vivo in mice.

We depleted different combinations of master regulators using lentiviral shRNA in xenograft tumors in mice. The control shRNA contained a scrambled sequence. These xenografts were derived from several GSC lines that have been labeled with a bioluminescent.

Recurrent tumors in experimental mice grew from cells that did not have master regulators depleted. This shows that efficient depletion is crucial.

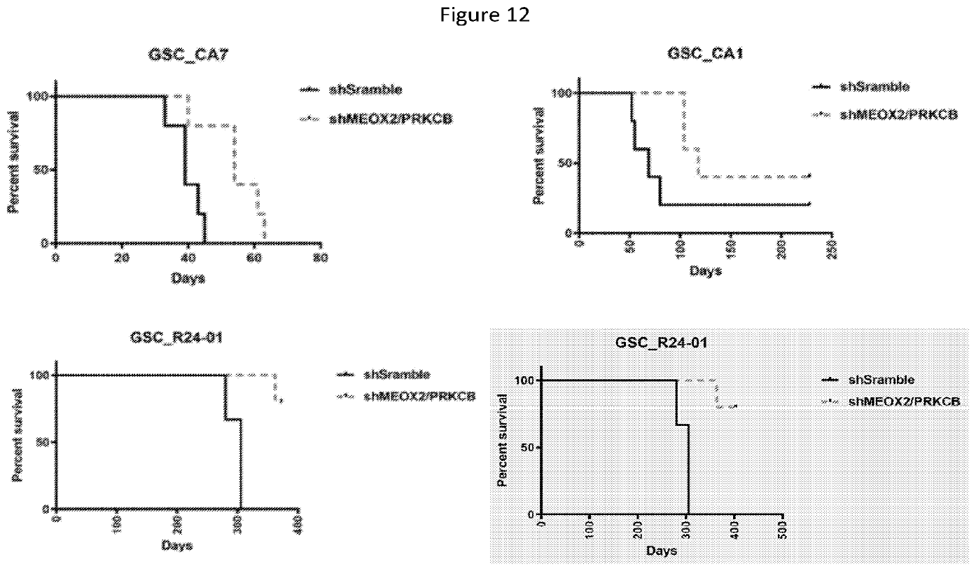

Results are shown in FIG. 12. MEOX2 and PRKCB showed increased survival. R24-01 is ongoing with all surviving mice showing no evidence of disease up to Day 450.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

NKX2-2 RNA
(SEQ ID NO: 1)
ccattttttc ctcgccacca gccgccaccg cgcgccgagc ggccgccgga gcccgagctg
acgccgcctt ggcacccctc ctggagttag aaactaaggc cggggcccgc ggcgctcggc
gcgcaggccg cccggcttcc tgcgtccatt tccgcgtgct ttcaaagaag acagagagag
gcactgggtt gggcttcatt tttttcctcc ccatccccag tttctttctc tttttaaaaa
taataattat cccaataatt aaagccaatt cccccctccc ctcccccagt ccctcccccc
aactcccccc tcccccgccc gccggggcag gggagcgcca cgaattgacc aagtgaagct
acaactttgc gacataaatt ttggggtctc gaaccatgtc gctgaccaac acaaagacgg
ggttttcggt caaggacatc ttagacctgc cggacaccaa cgatgaggag ggctctgtgg
ccgaaggtcc ggaggaagag aacgaggggc ccgagccagc caagagggcc gggccgctgg
ggcagggcgc cctggacgcg gtgcagagcc tgcccctgaa gaaccccttc tacgacagca
gcgacaaccc gtacacgcgc tggctggcca gcaccgaggg ccttcagtac tccctgcacg
gtctggctgc cggggcgccc cctcaggact caagctccaa gtccccggag ccctcggccg
acgagtcacc ggacaatgac aaggagaccc cgggcggcgg gggggacgcc ggcaagaagc
gaaagcggcg agtgcttttc tccaaggcgc agacctacga gctggagcgg cgctttcggc
agcagcggta cctgtcggcg cccgagcgcg aacacctggc cagcctcatc cgcctcacgc
ccacgcaggt caagatctgg ttccagaacc accgctacaa gatgaagcgc gcccgggccg
agaaaggtat ggaggtgacg cccctgccct cgccgcgccg ggtggccgtg cccgtcttgg
tcagggacgg caaaccatgt cacgcgctca aagcccagga cctggcagcc gccaccttcc
aggcgggcat tcccttttct gcctacagcg cgcagtcgct gcagcacatg cagtacaacg
cccagtacag ctcggccagc accccccagt acccgacagc acaccccctg gtccaggccc
agcagtggac ttggtgagcg ccgccccaac gagactcgcg gccccaggcc caggccccac
cccggcggcg gtggcggcga ggaggcctcg gtccttatgg tggttattat tattattata
attattatta tggagtcgag ttgactctcg gctccactag ggaggcgccg ggaggttgcc
tgcgtctcct tggagtggca gattccaccc acccagctct gcccatgcct ctccttctga
accttgggag agggctgaac tctacgccgt gtttacagaa tgtttgcgca gcttcgcttc
tttgcctctc cccgggggga ccaaaccgtc ccagcgttaa tgtcgtcact tgaaaacgag
aaaaagaccg accccccacc cctgctttcg tgcattttgt aaaatatgtt tgtgtgagta
gcgatattgt cagccgtctt ctaaagcaag tggagaacac tttaaaaata cagagaattt
cttccttttt ttaaaaaaaa ataagaaaat gctaaatatt tatggccatg taaacgttct
gacaactggt ggcagatttc gcttttcgtt gtaaatatcg gtggtgattg ttgccaaaat
gaccttcagg accggcctgt ttcccgtctg ggtccaactc ctttctttgt ggcttgtttg
ggtttgtttt ttgttttgtt tttgtttttg cgttttcccc tgctttcttc ctttctcttt
ttattttatt gtgcaaacat ttctcaaata tggaaaagaa aaccctgtag gcagggagcc
ctctgccctg tcctccgggc cttcagcccc gaacttggag ctcagctatt cggcgcggtt
ccccaacagc gccgggcgca gaaagctttc gattttttaa ataagaattt taataaaaat
cctgtgttta aaaaagaaaa aaa
NKX2-2 PROTEIN
(SEQ ID NO: 2)
MSLTNTKTGF SVKDILDLPD TNDEEGSVAE GPEEENEGPE PAKRAGPLGQ GALDAVQSLP
LKNPFYDSSD NPYTRWLAST EGLQYSLHGL AAGAPPQDSS SKSPEPSADE SPDNDKETPG
GGGDAGKKRK RRVLFSKAQT YELERRFRQQ RYLSAPEREH LASLIRLTPT QVKIWFQNHR
YKMKRARAEK GMEVTPLPSP RRVAVPVLVR DGKPCHALKA QDLAAATFQA GIPFSAYSAQ
SLQHMQYNAQ YSSASTPQYP TAHPLVQAQQ WTW
MLXIPL RNA Isoform alpha
(SEQ ID NO: 3)
agggaccagg cggttgcggc ggcgacagcc atggccggcg cgctggcagg tctggccgcg
ggcttgcagg tcccgcgggt cgcgcccagc ccagactcgg actcggacac agactcggag
gacccgagtc tccggcgcag cgcgggcggc ttgctccgct cgcaggtcat ccacagcggt
cacttcatgg tgtcgtcgcc gcacagcgac tcgctgcccc ggcggcgcga ccaggagggg
tccgtggggc cctccgactt cgggccgcgc agtatcgacc ccacactcac acgcctcttc
gagtgcttga gcctggccta cagtggcaag ctggtgtctc ccaagtggaa gaatttcaaa
ggcctcaagc tgctctgcag agacaagatc cgcctgaaca acgccatctg gagggcctgg
tatatccagt atgtgaagcg gaggaagagc cccgtgtgtg gcttcgtgac ccccctgcag
gggcctgagg ctgatgcgca ccggaagccg gaggccgtgg tcctggaggg gaactactgg
aagcggcgca tcgaggtggt gatgcgggaa taccacaagt ggcgcatcta ctacaagaag
cggctccgta agcccagcag ggaagatgac ctcctggccc ctaagcaggc ggaaggcagg
tggccgccgc cggagcaatg gtgcaaacag ctcttctcca gtgtggtccc cgtgctgctg
ggggacccag aggaggagcc gggtgggcgg cagctcctgg acctcaattg ctttttgtcc
gacatctcag acactctctt caccatgact cagtccggcc cttcgcccct gcagctgccg
cctgaggatg cctacgtcgg caatgctgac atgatccagc cggacctgac gccactgcag
ccaagcctgg atgacttcat ggacatctca gatttcttta ccaactcccg cctcccacag
ccgcccatgc cttcaaactt cccagagccc cccagcttca gccccgtggt tgactccctc
ttcagcagtg ggaccctggg cccagaggtg cccccggctt cctcggccat gacccacctc
tctggacaca gccgtctgca ggctcggaac agctgccctg gccccttgga ctccagcgcc
ttcctgagtt ctgatttcct ccttcctgaa gaccccaagc cccggctccc accccctcct
gtacccccac ctctgctgca ttaccctccc cctgccaagg tgccaggcct ggagccctgc
cccccacctc ccttccctcc catggcacca cccactgctt tgctgcagga agagcctctc
ttctctccca ggtttccctt ccccaccgtc cctcctgccc caggagtgtc tccgctgcct
gctcctgcag ccttcccacc caccccacag tctgtcccca gcccagcccc cacccccttc
cccatagagc ttctaccctt ggggtattcg gagcctgcct ttgggccttg cttctccatg
cccagaggca agccccccgc cccatcccct aggggacaga aagccagccc ccctacctta
gcccctgcca ctgccagtcc ccccaccact gcggggagca acaacccctg cctcacacag
ctgctcacag cagctaagcc ggagcaagcc ctggagccac cacttgtatc cagcaccctc
ctccggtccc cagggtcccc gcaggagaca gtccctgaat tcccctgcac attccttccc
ccgaccccgg cccctacacc gccccggcca cctccaggcc cggccacatt ggccccttcc
aggcccctgc ttgtccccaa agcggagcgg ctctcacccc cagcgcccag cggcagtgaa
cggcggctgt caggggacct cagctccatg ccaggccctg ggactctgag cgtccgtgtc
tctcccccgc aacccatcct cagccggggc cgtccagaca gcaacaagac cgagaaccgg
cgtatcacac acatctccgc ggagcagaag cggcgcttca acatcaagct ggggtttgac
acccttcatg ggctcgtgag cacactcagt gcccagccca gcctcaaggt gagcaaagct
accacgctgc agaagacagc tgagtacatc cttatgctac agcaggagcg tgcgggcttg
caggaggagg cccagcagct gcgggatgag attgaggagc tcaatgccgc cattaacctg
tgccagcagc agctgcccgc cacaggggta cccatcacac accagcgttt tgaccagatg
cgagacatgt ttgatgacta cgtccgaacc cgtacgctgc acaactggaa gttctgggtg
ttcagcatcc tcatccggcc tctgtttgag tccttcaacg ggatggtgtc cacggcaagt
gtgcacaccc tccgccagac ctcactggcc tggctggacc agtactgctc tctgcccgct
ctccggccaa ctgtcctgaa ctccctacgc cagctgggca catctaccag tatcctgacc
gacccgggcc gcatccctga gcaagccaca cgggcagtca cagagggcac ccttggcaaa
cctttatagt cctggccaga ccctgctgct cactcagctg ccctgggggc tgctttccct
gggcacgggc tccagggatc atctctgggc actcccttcc tgccccaggc cctggctctg
cccttccctg gggggtggag cagggtccag gtttcacact tgccacctcc tggaggtcaa
gaagagcaga gtccccgtcc ctgctctgcc actgtgctcc agcaccgtga ccttgggtga
ctcgtccgct gtctttggac cgctgtgttt caatctgcaa aatggggatg gggaaggttc
aatcagcaga tgacccccag gccttggcag ctgtgacatt gggggcctag gctggcaact
ccgggggctc aacggtggaa agaggaggat gctgtttctc tgtcacctcc acttgctccc
cgacaggtgg ggcacagacc tctgttcctg agcagagaag cagaaaagga ggttccctct
ctctgctcct tcactgctga cccagagggg ctgcaggatg gtttcccctg ggagaggcca
ggagggcctg atcccaggag acaccagggc cagagtgacc acagcagggc aggcatcatg
tgtgtgtgtg tgtgtggatg tgtgtgtgtg ggttttgtaa agaattcttg accaataaaa
gcaaaaactg tc
MLXIPL Protein Isoform alpha
(SEQ ID NO: 4)
MAGALAGLAAGLQVPRVAPSPDSDSDTDSEDPSLRRSAGGLLRS
QVIHSGHFMVSSPHSDSLPRRRDQEGSVGPSDFGPRSIDPTLTRLFECLSLAYSGKLV
SPKWKNFKGLKLLCRDKIRLNNAIWRAWYIQYVKRRKSPVCGFVTPLQGPEADAHRKP
EAVVLEGNYWKRRIEVVMREYHKWRIYYKKRLRKPSREDDLLAPKQAEGRWPPPEQWC
KQLFSSVVPVLLGDPEEEPGGRQLLDLNCFLSDISDTLFTMTQSGPSPLQLPPEDAYV
GNADMIQPDLTPLQPSLDDFMDISDFFTNSRLPQPPMPSNFPEPPSFSPVVDSLFSSG
TLGPEVPPASSAMTHLSGHSRLQARNSCPGPLDSSAFLSSDFLLPEDPKPRLPPPPVP
PPLLHYPPPAKVPGLEPCPPPPFPPMAPPTALLQEEPLFSPRFPFPTVPPAPGVSPLP
APAAFPPTPQSVPSPAPTPFPIELLPLGYSEPAFGPCFSMPRGKPPAPSPRGQKASPP
TLAPATASPPTTAGSNNPCLTQLLTAAKPEQALEPPLVSSTLLRSPGSPQETVPEFPC
TFLPPTPAPTPPRPPPGPATLAPSRPLLVPKAERLSPPAPSGSERRLSGDLSSMPGPG
TLSVRVSPPQPILSRGRPDSNKTENRRITHISAEQKRRFNIKLGFDTLHGLVSTLSAQ
PSLKVSKATTLQKTAEYILMLQQERAGLQEEAQQLRDEIEELNAAINLCQQQLPATGV
PITHQRFDQMRDMFDDYVRTRTLHNWKFWVFSILIRPLFESFNGMVSTASVHTLRQTS
LAWLDQYCSLPALRPTVLNSLRQLGTSTSILTDPGRIPEQATRAVTEGTLGKPL
MLXIPL RNA Isoform beta
(SEQ ID NO: 5)
agggaccagg cggttgcggc ggcgacagcc atggccggcg cgctggcagg tctggccgcg
ggcttgcagg tcccgcgggt cgcgcccagc ccagactcgg actcggacac agactcggag
gacccgagtc tccggcgcag cgcgggcggc ttgctccgct cgcaggtcat ccacagcggt
cacttcatgg tgtcgtcgcc gcacagcgac tcgctgcccc ggcggcgcga ccaggagggg
tccgtggggc cctccgactt cgggccgcgc agtatcgacc ccacactcac acgcctcttc
gagtgcttga gcctggccta cagtggcaag ctggtgtctc ccaagtggaa gaatttcaaa
ggcctcaagc tgctctgcag agacaagatc cgcctgaaca acgccatctg gagggcctgg
tatatccagt atgtgaagcg gaggaagagc cccgtgtgtg gcttcgtgac ccccctgcag
gggcctgagg ctgatgcgca ccggaagccg gaggccgtgg tcctggaggg gaactactgg
aagcggcgca tcgaggtggt gatgcgggaa taccacaagt ggcgcatcta ctacaagaag
cggctccgta agcccagcag ggaagatgac ctcctggccc ctaagcaggc ggaaggcagg
tggccgccgc cggagcaatg gtgcaaacag ctcttctcca gtgtggtccc cgtgctgctg
ggggacccag aggaggagcc gggtgggcgg cagctcctgg acctcaattg ctttttgtcc
gacatctcag acactctctt caccatgact cagtccggcc cttcgcccct gcagctgccg
cctgaggatg cctacgtcgg caatgctgac atgatccagc cggacctgac gccactgcag
ccaagcctgg atgacttcat ggacatctca gatttcttta ccaactcccg cctcccacag
ccgcccatgc cttcaaactt cccagagccc cccagcttca gccccgtggt tgactccctc
ttcagcagtg ggaccctggg cccagaggtg cccccggctt cctcggccat gacccacctc
tctggacaca gccgtctgca ggctcggaac agctgccctg gccccttgga ctccagcgcc
ttcctgagtt ctgatttcct ccttcctgaa gaccccaagc cccggctccc accccctcct
gtacccccac ctctgctgca ttaccctccc cctgccaagg tgccaggcct ggagccctgc
cccccacctc ccttccctcc catggcacca cccactgctt tgctgcagga agagcctctc
ttctctccca ggtttccctt ccccaccgtc cctcctgccc caggagtgtc tccgctgcct
gctcctgcag ccttcccacc caccccacag tctgtcccca gcccagcccc cacccccttc
cccatagagc ttctaccctt ggggtattcg gagcctgcct ttgggccttg cttctccatg
cccagaggca agccccccgc cccatcccct aggggacaga aagccagccc ccctacctta
gcccctgcca ctgccagtcc ccccaccact gcggggagca acaacccctg cctcacacag
ctgctcacag cagctaagcc ggagcaagcc ctggagccac cacttgtatc cagcaccctc
ctccggtccc cagggtcccc gcaggagaca gtccctgaat tcccctgcac attccttccc
ccgaccccgg cccctacacc gccccggcca cctccaggcc cggccacatt ggccccttcc
aggcccctgc ttgtccccaa agcggagcgg ctctcacccc cagcgcccag cggcagtgaa
cggcggctgt caggggacct cagctccatg ccaggccctg ggactctgag cgtccgtgtc
tctcccccgc aacccatcct cagccggggc cgtccagaca gcaacaagac cgagaaccgg
cgtatcacac acatctccgc ggagcagaag cggcgcttca acatcaagct ggggtttgac
acccttcatg ggctcgtgag cacactcagt gcccagccca gcctcaagga gcgtgcgggc
ttgcaggagg aggcccagca gctgcgggat gagattgagg agctcaatgc cgccattaac
ctgtgccagc agcagctgcc cgccacaggg gtacccatca cacaccagcg ttttgaccag
atgcgagaca tgtttgatga ctacgtccga acccgtacgc tgcacaactg gaagttctgg
gtgttcagca tcctcatccg gcctctgttt gagtccttca acgggatggt gtccacggca
agtgtgcaca ccctccgcca gacctcactg gcctggctgg accagtactg ctctctgccc
gctctccggc caactgtcct gaactcccta cgccagctgg gcacatctac cagtatcctg
accgacccgg gccgcatccc tgagcaagcc acacgggcag tcacagaggg cacccttggc
aaacctttat agtcctggcc agaccctgct gctcactcag ctgccctggg ggctgctttc
cctgggcacg ggctccaggg atcatctctg ggcactccct tcctgcccca ggccctggct
ctgcccttcc ctggggggtg gagcagggtc caggtttcac acttgccacc tcctggaggt
caagaagagc agagtccccg tccctgctct gccactgtgc tccagcaccg tgaccttggg
tgactcgtcc gctgtctttg gaccgctgtg tttcaatctg caaaatgggg atggggaagg
ttcaatcagc agatgacccc caggccttgg cagctgtgac attgggggcc taggctggca
actccggggg ctcaacggtg gaaagaggag gatgctgttt ctctgtcacc tccacttgct
ccccgacagg tggggcacag acctctgttc ctgagcagag aagcagaaaa ggaggttccc
tctctctgct ccttcactgc tgacccagag gggctgcagg atggtttccc ctgggagagg
ccaggagggc ctgatcccag gagacaccag ggccagagtg accacagcag ggcaggcatc
atgtgtgtgt gtgtgtgtgg atgtgtgtgt gtgggttttg taaagaattc ttgaccaata
aaagcaaaaa ctgtc
MLXIPL protein Isoform beta
(SEQ ID NO: 6)
MAGALAGLAAGLQVPRVAPSPDSDSDTDSEDPSLRRSAGGLLRS
QVIHSGHFMVSSPHSDSLPRRRDQEGSVGPSDFGPRSIDPTLTRLFECLSLAYSGKLV
SPKWKNFKGLKLLCRDKIRLNNAIWRAWYIQYVKRRKSPVCGFVTPLQGPEADAHRKP
EAVVLEGNYWKRRIEVVMREYHKWRIYYKKRLRKPSREDDLLAPKQAEGRWPPPEQWC
KQLFSSVVPVLLGDPEEEPGGRQLLDLNCFLSDISDTLFTMTQSGPSPLQLPPEDAYV
GNADMIQPDLTPLQPSLDDFMDISDFFTNSRLPQPPMPSNFPEPPSFSPVVDSLFSSG
TLGPEVPPASSAMTHLSGHSRLQARNSCPGPLDSSAFLSSDFLLPEDPKPRLPPPPVP
PPLLHYPPPAKVPGLEPCPPPPFPPMAPPTALLQEEPLFSPRFPFPTVPPAPGVSPLP
APAAFPPTPQSVPSPAPTPFPIELLPLGYSEPAFGPCFSMPRGKPPAPSPRGQKASPP
TLAPATASPPTTAGSNNPCLTQLLTAAKPEQALEPPLVSSTLLRSPGSPQETVPEFPC
TFLPPTPAPTPPRPPPGPATLAPSRPLLVPKAERLSPPAPSGSERRLSGDLSSMPGPG
TLSVRVSPPQPILSRGRPDSNKTENRRITHISAEQKRRFNIKLGFDTLHGLVSTLSAQ
PSLKERAGLQEEAQQLRDEIEELNAAINLCQQQLPATGVPITHQRFDQMRDMFDDYVR
TRTLHNWKFWVFSILIRPLFESFNGMVSTASVHTLRQTSLAWLDQYCSLPALRPTVLN
SLRQLGTSTSILTDPGRIPEQATRAVTEGTLGKPL
MLXIPL RNA Isoform gamma
(SEQ ID NO: 7)
agggaccagg cggttgcggc ggcgacagcc atggccggcg cgctggcagg tctggccgcg
ggcttgcagg tcccgcgggt cgcgcccagc ccagactcgg actcggacac agactcggag
gacccgagtc tccggcgcag cgcgggcggc ttgctccgct cgcaggtcat ccacagcggt
cacttcatgg tgtcgtcgcc gcacagcgac tcgctgcccc ggcggcgcga ccaggagggg
tccgtggggc cctccgactt cgggccgcgc agtatcgacc ccacactcac acgcctcttc
gagtgcttga gcctggccta cagtggcaag ctggtgtctc ccaagtggaa gaatttcaaa
ggcctcaagc tgctctgcag agacaagatc cgcctgaaca acgccatctg gagggcctgg
tatatccagt atgtgaagcg gaggaagagc cccgtgtgtg gcttcgtgac ccccctgcag
gggcctgagg ctgatgcgca ccggaagccg gaggccgtgg tcctggaggg gaactactgg
aagcggcgca tcgaggtggt gatgcgggaa taccacaagt ggcgcatcta ctacaagaag
cggctccgta agcccagcag ggaagatgac ctcctggccc ctaagcaggc ggaaggcagg
tggccgccgc cggagcaatg gtgcaaacag ctcttctcca gtgtggtccc cgtgctgctg
ggggacccag aggaggagcc gggtgggcgg cagctcctgg acctcaattg ctttttgtcc
gacatctcag acactctctt caccatgact cagtccggcc cttcgcccct gcagctgccg
cctgaggatg cctacgtcgg caatgctgac atgatccagc cggacctgac gccactgcag
ccaagcctgg atgacttcat ggacatctca gatttcttta ccaactcccg cctcccacag
ccgcccatgc cttcaaactt cccagagccc cccagcttca gccccgtggt tgactccctc
ttcagcagtg ggaccctggg cccagaggtg cccccggctt cctcggccat gacccacctc
tctggacaca gccgtctgca ggctcggaac agctgccctg gccccttgga ctccagcgcc
ttcctgagtt ctgatttcct ccttcctgaa gaccccaagc cccggctccc accccctcct
gtacccccac ctctgctgca ttaccctccc cctgccaagg tgccaggcct ggagccctgc
cccccacctc ccttccctcc catggcacca cccactgctt tgctgcagga agagcctctc
ttctctccca ggtttccctt ccccaccgtc cctcctgccc caggagtgtc tccgctgcct
gctcctgcag ccttcccacc caccccacag tctgtcccca gcccagcccc cacccccttc
cccatagagc ttctaccctt ggggtattcg gagcctgcct ttgggccttg cttctccatg
cccagaggca agccccccgc cccatcccct aggggacaga aagccagccc ccctacctta
gcccctgcca ctgccagtcc ccccaccact gcggggagca acaacccctg cctcacacag
ctgctcacag cagctaagcc ggagcaagcc ctggagccac cacttgtatc cagcaccctc
ctccggtccc cagggtcccc gcaggagaca gtccctgaat tcccctgcac attccttccc
ccgaccccgg cccctacacc gccccggcca cctccaggcc cggccacatt ggccccttcc
aggcccctgc ttgtccccaa agcggagcgg ctctcacccc cagcgcccag cggcagtgaa
cggcggctgt caggggacct cagctccatg ccaggccctg ggactctgag cgtccgtgtc
tctcccccgc aacccatcct cagccggggc cgtccagaca gcaacaagaa ccggcgtatc
acacacatct ccgcggagca gaagcggcgc ttcaacatca agctggggtt tgacaccctt
catgggctcg tgagcacact cagtgcccag cccagcctca aggtgagcaa agctaccacg
ctgcagaaga cagctgagta catccttatg ctacagcagg agcgtgcggg cttgcaggag
gaggcccagc agctgcggga tgagattgag gagctcaatg ccgccattaa cctgtgccag
cagcagctgc ccgccacagg ggtacccatc acacaccagc gttttgacca gatgcgagac
atgtttgatg actacgtccg aacccgtacg ctgcacaact ggaagttctg ggtgttcagc
atcctcatcc ggcctctgtt tgagtccttc aacgggatgg tgtccacggc aagtgtgcac
accctccgcc agacctcact ggcctggctg gaccagtact gctctctgcc cgctctccgg
ccaactgtcc tgaactccct acgccagctg ggcacatcta ccagtatcct gaccgacccg
ggccgcatcc ctgagcaagc cacacgggca gtcacagagg gcacccttgg caaaccttta
tagtcctggc cagaccctgc tgctcactca gctgccctgg gggctgcttt ccctgggcac
gggctccagg gatcatctct gggcactccc ttcctgcccc aggccctggc tctgcccttc
cctggggggt ggagcagggt ccaggtttca cacttgccac ctcctggagg tcaagaagag
cagagtcccc gtccctgctc tgccactgtg ctccagcacc gtgaccttgg gtgactcgtc
cgctgtcttt ggaccgctgt gtttcaatct gcaaaatggg gatggggaag gttcaatcag
cagatgaccc ccaggccttg gcagctgtga cattgggggc ctaggctggc aactccgggg
gctcaacggt ggaaagagga ggatgctgtt tctctgtcac ctccacttgc tccccgacag
gtggggcaca gacctctgtt cctgagcaga gaagcagaaa aggaggttcc ctctctctgc
tccttcactg ctgacccaga ggggctgcag gatggtttcc cctgggagag gccaggaggg
cctgatccca ggagacacca gggccagagt gaccacagca gggcaggcat catgtgtgtg
tgtgtgtgtg gatgtgtgtg tgtgggtttt gtaaagaatt cttgaccaat aaaagcaaaa
actgtc
MLXIPL Protein Isoform gamma
(SEQ ID NO: 8)
MAGALAGLAAGLQVPRVAPSPDSDSDTDSEDPSLRRSAGGLLRS
QVIHSGHFMVSSPHSDSLPRRRDQEGSVGPSDFGPRSIDPTLTRLFECLSLAYSGKLV
SPKWKNFKGLKLLCRDKIRLNNAIWRAWYIQYVKRRKSPVCGFVTPLQGPEADAHRKP
EAVVLEGNYWKRRIEVVMREYHKWRIYYKKRLRKPSREDDLLAPKQAEGRWPPPEQWC
KQLFSSVVPVLLGDPEEEPGGRQLLDLNCFLSDISDTLFTMTQSGPSPLQLPPEDAYV
GNADMIQPDLTPLQPSLDDFMDISDFFTNSRLPQPPMPSNFPEPPSFSPVVDSLFSSG
TLGPEVPPASSAMTHLSGHSRLQARNSCPGPLDSSAFLSSDFLLPEDPKPRLPPPPVP
PPLLHYPPPAKVPGLEPCPPPPFPPMAPPTALLQEEPLFSPRFPFPTVPPAPGVSPLP
APAAFPPTPQSVPSPAPTPFPIELLPLGYSEPAFGPCFSMPRGKPPAPSPRGQKASPP
TLAPATASPPTTAGSNNPCLTQLLTAAKPEQALEPPLVSSTLLRSPGSPQETVPEFPC
TFLPPTPAPTPPRPPPGPATLAPSRPLLVPKAERLSPPAPSGSERRLSGDLSSMPGPG
TLSVRVSPPQPILSRGRPDSNKNRRITHISAEQKRRFNIKLGFDTLHGLVSTLSAQPS
LKVSKATTLQKTAEYILMLQQERAGLQEEAQQLRDEIEELNAAINLCQQQLPATGVPI
THQRFDQMRDMFDDYVRTRTLHNWKFWVFSILIRPLFESFNGMVSTASVHTLRQTSLA
WLDQYCSLPALRPTVLNSLRQLGTSTSILTDPGRIPEQATRAVTEGTLGKPL
MLXIPL RNA Isoform delta
(SEQ ID NO: 9)
agggaccagg cggttgcggc ggcgacagcc atggccggcg cgctggcagg tctggccgcg
ggcttgcagg tcccgcgggt cgcgcccagc ccagactcgg actcggacac agactcggag
gacccgagtc tccggcgcag cgcgggcggc ttgctccgct cgcaggtcat ccacagcggt
cacttcatgg tgtcgtcgcc gcacagcgac tcgctgcccc ggcggcgcga ccaggagggg
tccgtggggc cctccgactt cgggccgcgc agtatcgacc ccacactcac acgcctcttc
gagtgcttga gcctggccta cagtggcaag ctggtgtctc ccaagtggaa gaatttcaaa
ggcctcaagc tgctctgcag agacaagatc cgcctgaaca acgccatctg gagggcctgg
tatatccagt atgtgaagcg gaggaagagc cccgtgtgtg gcttcgtgac ccccctgcag
gggcctgagg ctgatgcgca ccggaagccg gaggccgtgg tcctggaggg gaactactgg
aagcggcgca tcgaggtggt gatgcgggaa taccacaagt ggcgcatcta ctacaagaag
cggctccgta agcccagcag ggaagatgac ctcctggccc ctaagcaggc ggaaggcagg
tggccgccgc cggagcaatg gtgcaaacag ctcttctcca gtgtggtccc cgtgctgctg
ggggacccag aggaggagcc gggtgggcgg cagctcctgg acctcaattg ctttttgtcc
gacatctcag acactctctt caccatgact cagtccggcc cttcgcccct gcagctgccg
cctgaggatg cctacgtcgg caatgctgac atgatccagc cggacctgac gccactgcag
ccaagcctgg atgacttcat ggacatctca gatttcttta ccaactcccg cctcccacag
ccgcccatgc cttcaaactt cccagagccc cccagcttca gccccgtggt tgactccctc
ttcagcagtg ggaccctggg cccagaggtg cccccggctt cctcggccat gacccacctc
tctggacaca gccgtctgca ggctcggaac agctgccctg gccccttgga ctccagcgcc
ttcctgagtt ctgatttcct ccttcctgaa gaccccaagc cccggctccc accccctcct
gtacccccac ctctgctgca ttaccctccc cctgccaagg tgccaggcct ggagccctgc
cccccacctc ccttccctcc catggcacca cccactgctt tgctgcagga agagcctctc
ttctctccca ggtttccctt ccccaccgtc cctcctgccc caggagtgtc tccgctgcct
gctcctgcag ccttcccacc caccccacag tctgtcccca gcccagcccc cacccccttc
cccatagagc ttctaccctt ggggtattcg gagcctgcct ttgggccttg cttctccatg
cccagaggca agccccccgc cccatcccct aggggacaga aagccagccc ccctacctta
gcccctgcca ctgccagtcc ccccaccact gcggggagca acaacccctg cctcacacag
ctgctcacag cagctaagcc ggagcaagcc ctggagccac cacttgtatc cagcaccctc
ctccggtccc cagggtcccc gcaggagaca gtccctgaat tcccctgcac attccttccc
ccgaccccgg cccctacacc gccccggcca cctccaggcc cggccacatt ggccccttcc
aggcccctgc ttgtccccaa agcggagcgg ctctcacccc cagcgcccag cggcagtgaa
cggcggctgt caggggacct cagctccatg ccaggccctg ggactctgag cgtccgtgtc
tctcccccgc aacccatcct cagccggggc cgtccagaca gcaacaagaa ccggcgtatc
acacacatct ccgcggagca gaagcggcgc ttcaacatca agctggggtt tgacaccctt
catgggctcg tgagcacact cagtgcccag cccagcctca aggagcgtgc gggcttgcag
gaggaggccc agcagctgcg ggatgagatt gaggagctca atgccgccat taacctgtgc
cagcagcagc tgcccgccac aggggtaccc atcacacacc agcgttttga ccagatgcga
gacatgtttg atgactacgt ccgaacccgt acgctgcaca actggaagtt ctgggtgttc
agcatcctca tccggcctct gtttgagtcc ttcaacggga tggtgtccac ggcaagtgtg
cacaccctcc gccagacctc actggcctgg ctggaccagt actgctctct gcccgctctc
cggccaactg tcctgaactc cctacgccag ctgggcacat ctaccagtat cctgaccgac
ccgggccgca tccctgagca agccacacgg gcagtcacag agggcaccct tggcaaacct
ttatagtcct ggccagaccc tgctgctcac tcagctgccc tgggggctgc tttccctggg
cacgggctcc agggatcatc tctgggcact cccttcctgc cccaggccct ggctctgccc
ttccctgggg ggtggagcag ggtccaggtt tcacacttgc cacctcctgg aggtcaagaa
gagcagagtc cccgtccctg ctctgccact gtgctccagc accgtgacct tgggtgactc
gtccgctgtc tttggaccgc tgtgtttcaa tctgcaaaat ggggatgggg aaggttcaat
cagcagatga cccccaggcc ttggcagctg tgacattggg ggcctaggct ggcaactccg
ggggctcaac ggtggaaaga ggaggatgct gtttctctgt cacctccact tgctccccga
caggtggggc acagacctct gttcctgagc agagaagcag aaaaggaggt tccctctctc
tgctccttca ctgctgaccc agaggggctg caggatggtt tcccctggga gaggccagga
gggcctgatc ccaggagaca ccagggccag agtgaccaca gcagggcagg catcatgtgt
gtgtgtgtgt gtggatgtgt gtgtgtgggt tttgtaaaga attcttgacc aataaaagca
aaaactgtc
MLXIPL Protein Isoform delta
(SEQ ID NO: 10)
MAGALAGLAAGLQVPRVAPSPDSDSDTDSEDPSLRRSAGGLLRS
QVIHSGHFMVSSPHSDSLPRRRDQEGSVGPSDFGPRSIDPTLTRLFECLSLAYSGKLV
SPKWKNFKGLKLLCRDKIRLNNAIWRAWYIQYVKRRKSPVCGFVTPLQGPEADAHRKP
EAVVLEGNYWKRRIEVVMREYHKWRIYYKKRLRKPSREDDLLAPKQAEGRWPPPEQWC
KQLFSSVVPVLLGDPEEEPGGRQLLDLNCFLSDISDTLFTMTQSGPSPLQLPPEDAYV
GNADMIQPDLTPLQPSLDDFMDISDFFTNSRLPQPPMPSNFPEPPSFSPVVDSLFSSG
TLGPEVPPASSAMTHLSGHSRLQARNSCPGPLDSSAFLSSDFLLPEDPKPRLPPPPVP
PPLLHYPPPAKVPGLEPCPPPPFPPMAPPTALLQEEPLFSPRFPFPTVPPAPGVSPLP
APAAFPPTPQSVPSPAPTPFPIELLPLGYSEPAFGPCFSMPRGKPPAPSPRGQKASPP
TLAPATASPPTTAGSNNPCLTQLLTAAKPEQALEPPLVSSTLLRSPGSPQETVPEFPC
TFLPPTPAPTPPRPPPGPATLAPSRPLLVPKAERLSPPAPSGSERRLSGDLSSMPGPG
TLSVRVSPPQPILSRGRPDSNKNRRITHISAEQKRRFNIKLGFDTLHGLVSTLSAQPS
LKERAGLQEEAQQLRDEIEELNAAINLCQQQLPATGVPITHQRFDQMRDMFDDYVRTR
TLHNWKFWVFSILIRPLFESFNGMVSTASVHTLRQTSLAWLDQYCSLPALRPTVLNSL
RQLGTSTSILTDPGRIPEQATRAVTEGTLGKPL
ETV4 RNA isoform 1
(SEQ ID NO: 11)
gctcacaact gtctgctgcg cccgaaaaac aagtcggtgc gctggggacc cggggccggg
gccgccttac tccggcctag ccccgcggcc ctcggtgcgg gctccagggc atgctcggga
ccccccgcgg ctccagccca gacgccccgg cctcaggtct cggcccccgc ttggggcccc
ggccgtgcgg ccggagggag cggccggatg gagcggagga tgaaagccgg atacttggac
cagcaagtgc cctacacctt cagcagcaaa tcgcccggaa atgggagctt gcgcgaagcg
ctgatcggcc cgctggggaa gctcatggac ccgggctccc tgccgcccct cgactctgaa
gatctcttcc aggatctaag tcacttccag gagacgtggc tcgctgaagc tcaggtacca
gacagtgatg agcagtttgt tcctgatttc cattcagaaa acctagcttt ccacagcccc
accaccagga tcaagaagga gccccagagt ccccgcacag acccggccct gtcctgcagc
aggaagccgc cactccccta ccaccatggc gagcagtgcc tttactccag tgcctatgac
ccccccagac aaatcgccat caagtcccct gcccctggtg cccttggaca gtcgccccta
cagccctttc cccgggcaga gcaacggaat ttcctgagat cctctggcac ctcccagccc
caccctggcc atgggtacct cggggaacat agctccgtct tccagcagcc cctggacatt
tgccactcct tcacatctca gggagggggc cgggaacccc tcccagcccc ctaccaacac
cagctgtcgg agccctgccc accctatccc cagcagagct ttaagcaaga ataccatgat
cccctgtatg aacaggcggg ccagccagcc gtggaccagg gtggggtcaa tgggcacagg
tacccagggg cgggggtggt gatcaaacag gaacagacgg acttcgccta cgactcagat
gtcaccgggt gcgcatcaat gtacctccac acagagggct tctctgggcc ctctccaggt
gacggggcca tgggctatgg ctatgagaaa cctctgcgac cattcccaga tgatgtctgc
gttgtccctg agaaatttga aggagacatc aagcaggaag gggtcggtgc atttcgagag
gggccgccct accagcgccg gggtgccctg cagctgtggc aatttctggt ggccttgctg
gatgacccaa caaatgccca tttcattgcc tggacgggcc ggggaatgga gttcaagctc
attgagcctg aggaggtcgc caggctctgg ggcatccaga agaaccggcc agccatgaat
tacgacaagc tgagccgctc gctccgatac tattatgaga aaggcatcat gcagaaggtg
gctggtgagc gttacgtgta caagtttgtg tgtgagcccg aggccctctt ctctttggcc
ttcccggaca atcagcgtcc agctctcaag gctgagtttg accggcctgt cagtgaggag
gacacagtcc ctttgtccca cttggatgag agccccgcct acctcccaga gctggctggc
cccgcccagc catttggccc caagggtggc tactcttact agcccccagc ggctgttccc
cctgccgcag gtgggtgctg ccctgtgtac atataaatga atctggtgtt ggggaaacct
tcatctgaaa cccacagatg tctctggggc agatccccac tgtcctacca gttgccctag
cccagactct gagctgctca ccggagtcat tgggaaggaa aagtggagaa atggcaagtc
tagagtctca gaaactcccc tgggggtttc acctgggccc tggaggaatt cagctcagct
tcttcctagg tccaagcccc ccacaccttt tccccaacca cagagaacaa gagtttgttc
tgttctgggg gacagagaag gcgcttccca acttcatact ggcaggaggg tgaggaggtt
cactgagctc cccagatctc ccactgcggg gagacagaag cctggactct gccccacgct
gtggccctgg agggtcccgg tttgtcagtt cttggtgctc tgtgttccca gaggcaggcg
gaggttgaag aaaggaacct gggatgaggg gtgctgggta taagcagaga gggatgggtt
cctgctccaa gggacccttt gcctttcttc tgccctttcc taggcccagg cctgggtttg
tacttccacc tccaccacat ctgccagacc ttaataaagg cccccacttc tccca
ETV4 protein isoform 1
(SEQ ID NO: 12)
MERRMKAGYLDQQVPYTFSSKSPGNGSLREALIGPLGKLMDPGS
LPPLDSEDLFQDLSHFQETWLAEAQVPDSDEQFVPDFHSENLAFHSPTTRIKKEPQSP
RTDPALSCSRKPPLPYHHGEQCLYSSAYDPPRQIAIKSPAPGALGQSPLQPFPRAEQR
NFLRSSGTSQPHPGHGYLGEHSSVFQQPLDICHSFTSQGGGREPLPAPYQHQLSEPCP
PYPQQSFKQEYHDPLYEQAGQPAVDQGGVNGHRYPGAGVVIKQEQTDFAYDSDVTGCA
SMYLHTEGFSGPSPGDGAMGYGYEKPLRPFPDDVCVVPEKFEGDIKQEGVGAFREGPP
YQRRGALQLWQFLVALLDDPTNAHFIAWTGRGMEFKLIEPEEVARLWGIQKNRPAMNY
DKLSRSLRYYYEKGIMQKVAGERYVYKFVCEPEALFSLAFPDNQRPALKAEFDRPVSE
EDTVPLSHLDESPAYLPELAGPAQPFGPKGGYSY
ETV4 RNA isoform 2
(SEQ ID NO: 13)
gctcacaact gtctgctgcg cccgaaaaac aagtcggtgc gctggggacc cggggccggg
gccgccttac tccggcctag ccccgcggcc ctcggtgcgg gctccagggc atgctcggga
ccccccgcgg ctccagccca gacgccccgg cctcagaaat cgcccggaaa tgggagcttg
cgcgaagcgc tgatcggccc gctggggaag ctcatggacc cgggctccct gccgcccctc
gactctgaag atctcttcca ggatctaagt cacttccagg agacgtggct cgctgaagct
caggtaccag acagtgatga gcagtttgtt cctgatttcc attcagaaaa cctagctttc
cacagcccca ccaccaggat caagaaggag ccccagagtc cccgcacaga cccggccctg
tcctgcagca ggaagccgcc actcccctac caccatggcg agcagtgcct ttactccagt
gcctatgacc cccccagaca aatcgccatc aagtcccctg cccctggtgc ccttggacag
tcgcccctac agccctttcc ccgggcagag caacggaatt tcctgagatc ctctggcacc
tcccagcccc accctggcca tgggtacctc ggggaacata gctccgtctt ccagcagccc
ctggacattt gccactcctt cacatctcag ggagggggcc gggaacccct cccagccccc
taccaacacc agctgtcgga gccctgccca ccctatcccc agcagagctt taagcaagaa
taccatgatc ccctgtatga acaggcgggc cagccagccg tggaccaggg tggggtcaat
gggcacaggt acccaggggc gggggtggtg atcaaacagg aacagacgga cttcgcctac
gactcagatg tcaccgggtg cgcatcaatg tacctccaca cagagggctt ctctgggccc
tctccaggtg acggggccat gggctatggc tatgagaaac ctctgcgacc attcccagat
gatgtctgcg ttgtccctga gaaatttgaa ggagacatca agcaggaagg ggtcggtgca
tttcgagagg ggccgcccta ccagcgccgg ggtgccctgc agctgtggca atttctggtg
gccttgctgg atgacccaac aaatgcccat ttcattgcct ggacgggccg gggaatggag
ttcaagctca ttgagcctga ggaggtcgcc aggctctggg gcatccagaa gaaccggcca
gccatgaatt acgacaagct gagccgctcg ctccgatact attatgagaa aggcatcatg
cagaaggtgg ctggtgagcg ttacgtgtac aagtttgtgt gtgagcccga ggccctcttc
tctttggcct tcccggacaa tcagcgtcca gctctcaagg ctgagtttga ccggcctgtc
agtgaggagg acacagtccc tttgtcccac ttggatgaga gccccgccta cctcccagag
ctggctggcc ccgcccagcc atttggcccc aagggtggct actcttacta gcccccagcg
gctgttcccc ctgccgcagg tgggtgctgc cctgtgtaca tataaatgaa tctggtgttg
gggaaacctt catctgaaac ccacagatgt ctctggggca gatccccact gtcctaccag
ttgccctagc ccagactctg agctgctcac cggagtcatt gggaaggaaa agtggagaaa
tggcaagtct agagtctcag aaactcccct gggggtttca cctgggccct ggaggaattc
agctcagctt cttcctaggt ccaagccccc cacacctttt ccccaaccac agagaacaag
agtttgttct gttctggggg acagagaagg cgcttcccaa cttcatactg gcaggagggt
gaggaggttc actgagctcc ccagatctcc cactgcgggg agacagaagc ctggactctg
ccccacgctg tggccctgga gggtcccggt ttgtcagttc ttggtgctct gtgttcccag
aggcaggcgg aggttgaaga aaggaacctg ggatgagggg tgctgggtat aagcagagag
ggatgggttc ctgctccaag ggaccctttg cctttcttct gccctttcct aggcccaggc
ctgggtttgt acttccacct ccaccacatc tgccagacct taataaaggc ccccacttct
ccca
ETV4 protein isoform 2
(SEQ ID NO: 14)
MDPGSLPPLDSEDLFQDLSHFQETWLAEAQVPDSDEQFVPDFHS
ENLAFHSPTTRIKKEPQSPRTDPALSCSRKPPLPYHHGEQCLYSSAYDPPRQIAIKSP
APGALGQSPLQPFPRAEQRNFLRSSGTSQPHPGHGYLGEHSSVFQQPLDICHSFISQG
GGREPLPAPYQHQLSEPCPPYPQQSFKQEYHDPLYEQAGQPAVDQGGVNGHRYPGAGV
VIKQEQTDFAYDSDVTGCASMYLHTEGFSGPSPGDGAMGYGYEKPLRPFPDDVCVVPE
KFEGDIKQEGVGAFREGPPYQRRGALQLWQFLVALLDDPTNAHFIAWTGRGMEFKLIE
PEEVARLWGIQKNRPAMNYDKLSRSLRYYYEKGIMQKVAGERYVYKFVCEPEALFSLA
FPDNQRPALKAEFDRPVSEEDTVPLSHLDESPAYLPELAGPAQPFGPKGGYSY
ETV4 RNA isoform 3
(SEQ ID NO: 15)
gcttgcccag cccccgctgc tgccttccat ggcctcagcc gcagccctca agttgaggag
gggttccagc atcacactcc ctctgggtga actttccctg ggattttgtg gttggcaggc
aacctgggca aagaacagtc accaggaagc aggctggaag gaagaaattc ttgaatgtgg
ataggacttc ctcctcccct gccctcgagc tccaccccaa gccacttctc acatcacccc
ttcttccccc acagatgtca ccgggtgcgc atcaatgtac ctccacacag agggcttctc
tgggccctct ccaggtgacg gggccatggg ctatggctat gagaaacctc tgcgaccatt
cccagatgat gtctgcgttg tccctgagaa atttgaagga gacatcaagc aggaaggggt
cggtgcattt cgagaggggc cgccctacca gcgccggggt gccctgcagc tgtggcaatt
tctggtggcc ttgctggatg acccaacaaa tgcccatttc attgcctgga cgggccgggg
aatggagttc aagctcattg agcctgagga ggtcgccagg ctctggggca tccagaagaa
ccggccagcc atgaattacg acaagctgag ccgctcgctc cgatactatt atgagaaagg
catcatgcag aaggtggctg gtgagcgtta cgtgtacaag tttgtgtgtg agcccgaggc
cctcttctct ttggccttcc cggacaatca gcgtccagct ctcaaggctg agtttgaccg
gcctgtcagt gaggaggaca cagtcccttt gtcccacttg gatgagagcc ccgcctacct
cccagagctg gctggccccg cccagccatt tggccccaag ggtggctact cttactagcc
cccagcggct gttccccctg ccgcaggtgg gtgctgccct gtgtacatat aaatgaatct
ggtgttgggg aaaccttcat ctgaaaccca cagatgtctc tggggcagat ccccactgtc
ctaccagttg ccctagccca gactctgagc tgctcaccgg agtcattggg aaggaaaagt
ggagaaatgg caagtctaga gtctcagaaa ctcccctggg ggtttcacct gggccctgga
ggaattcagc tcagcttctt cctaggtcca agccccccac accttttccc caaccacaga
gaacaagagt ttgttctgtt ctgggggaca gagaaggcgc ttcccaactt catactggca
ggagggtgag gaggttcact gagctcccca gatctcccac tgcggggaga cagaagcctg
gactctgccc cacgctgtgg ccctggaggg tcccggtttg tcagttcttg gtgctctgtg
ttcccagagg caggcggagg ttgaagaaag gaacctggga tgaggggtgc tgggtataag
cagagaggga tgggttcctg ctccaaggga ccctttgcct ttcttctgcc ctttcctagg
cccaggcctg ggtttgtact tccacctcca ccacatctgc cagaccttaa taaaggcccc
cacttctccc a
ETV4 protein isoform 3
(SEQ ID NO: 16)
MYLHTEGFSGPSPGDGAMGYGYEKPLRPFPDDVCVVPEKFEGDI
KQEGVGAFREGPPYQRRGALQLWQFLVALLDDPINAHFIAWTGRGMEFKLIEPEEVAR
LWGIQKNRPAMNYDKLSRSLRYYYEKGIMQKVAGERYVYKFVCEPEALFSLAFPDNQR
PALKAEFDRPVSEEDTVPLSHLDESPAYLPELAGPAQPFGPKGGYSY
ETV4 RNA isoform 4
(SEQ ID NO: 17)
gcagaaagca gaaacggcga gcccggctcc tgggagcagg tctcggcccc cgcttggggc
cccggccgtg cggccggagg gagcggccgg atggagcgga ggatgaaagc cggatacttg
gaccagcaag tgccctacac cttcagcagc aaatcgcccg gaaatgggag cttgcgcgaa
gcgctgatcg gcccgctggg gaagctcatg gacccgggct ccctgccgcc cctcgactct
gaagatctct tccaggatct aagtcacttc caggagacgt ggctcgctga agctcaggta
ccagacagtg atgagcagtt tgttcctgat ttccattcag aaaacccttt ccacagcccc
accaccagga tcaagaagga gccccagagt ccccgcacag acccggccct gtcctgcagc
aggaagccgc cactccccta ccaccatggc gagcagtgcc tttactccag tgcctatgac
ccccccagac aaatcgccat caagtcccct gcccctggtg cccttggaca gtcgccccta
cagccctttc cccgggcaga gcaacggaat ttcctgagat cctctggcac ctcccagccc
caccctggcc atgggtacct cggggaacat agctccgtct tccagcagcc cctggacatt
tgccactcct tcacatctca gggagggggc cgggaacccc tcccagcccc ctaccaacac
cagctgtcgg agccctgccc accctatccc cagcagagct ttaagcaaga ataccatgat
cccctgtatg aacaggcggg ccagccagcc gtggaccagg gtggggtcaa tgggcacagg
tacccagggg cgggggtggt gatcaaacag gaacagacgg acttcgccta cgactcagat
gtcaccgggt gcgcatcaat gtacctccac acagagggct tctctgggcc ctctccaggt
gacggggcca tgggctatgg ctatgagaaa cctctgcgac cattcccaga tgatgtctgc
gttgtccctg agaaatttga aggagacatc aagcaggaag gggtcggtgc atttcgagag
gggccgccct accagcgccg gggtgccctg cagctgtggc aatttctggt ggccttgctg
gatgacccaa caaatgccca tttcattgcc tggacgggcc ggggaatgga gttcaagctc
attgagcctg aggaggtcgc caggctctgg ggcatccaga agaaccggcc agccatgaat
tacgacaagc tgagccgctc gctccgatac tattatgaga aaggcatcat gcagaaggtg
gctggtgagc gttacgtgta caagtttgtg tgtgagcccg aggccctctt ctctttggcc
ttcccggaca atcagcgtcc agctctcaag gctgagtttg accggcctgt cagtgaggag
gacacagtcc ctttgtccca cttggatgag agccccgcct acctcccaga gctggctggc
cccgcccagc catttggccc caagggtggc tactcttact agcccccagc ggctgttccc
cctgccgcag gtgggtgctg ccctgtgtac atataaatga atctggtgtt ggggaaacct
tcatctgaaa cccacagatg tctctggggc agatccccac tgtcctacca gttgccctag
cccagactct gagctgctca ccggagtcat tgggaaggaa aagtggagaa atggcaagtc
tagagtctca gaaactcccc tgggggtttc acctgggccc tggaggaatt cagctcagct
tcttcctagg tccaagcccc ccacaccttt tccccaacca cagagaacaa gagtttgttc
tgttctgggg gacagagaag gcgcttccca acttcatact ggcaggaggg tgaggaggtt
cactgagctc cccagatctc ccactgcggg gagacagaag cctggactct gccccacgct
gtggccctgg agggtcccgg tttgtcagtt cttggtgctc tgtgttccca gaggcaggcg
gaggttgaag aaaggaacct gggatgaggg gtgctgggta taagcagaga gggatgggtt
cctgctccaa gggacccttt gcctttcttc tgccctttcc taggcccagg cctgggtttg
tacttccacc tccaccacat ctgccagacc ttaataaagg cccccacttc tccca
ETV4 potein isoform 4
(SEQ ID NO: 18)
MERRMKAGYLDQQVPYTFSSKSPGNGSLREALIGPLGKLMDPGS
LPPLDSEDLFQDLSHFQETWLAEAQVPDSDEQFVPDFHSENPFHSPTTRIKKEPQSPR
TDPALSCSRKPPLPYHHGEQCLYSSAYDPPRQIAIKSPAPGALGQSPLQPFPRAEQRN
FLRSSGTSQPHPGHGYLGEHSSVFQQPLDICHSFTSQGGGREPLPAPYQHQLSEPCPP
YPQQSFKQEYHDPLYEQAGQPAVDQGGVNGHRYPGAGVVIKQEQTDFAYDSDVTGCAS
MYLHTEGFSGPSPGDGAMGYGYEKPLRPFPDDVCVVPEKFEGDIKQEGVGAFREGPPY
QRRGALQLWQFLVALLDDPTNAHFIAWTGRGMEFKLIEPEEVARLWGIQKNRPAMNYD
KLSRSLRYYYEKGIMQKVAGERYVYKFVCEPEALFSLAFPDNQRPALKAEFDRPVSEE
DTVPLSHLDESPAYLPELAGPAQPFGPKGGYSY
ETV4 RNA isoform 5
(SEQ ID NO: 19)
gcagaaagca gaaacggcga gcccggctcc tgggagcagg tctcggcccc cgcttggggc
cccggccgtg cggccggagg gagcggccgg atggagcgga ggatgaaagc cggatacttg
gaccagcaag tgccctacac cttcagcagc aaatcgcccg gaaatgggag cttgcgcgaa
gcgctgatcg gcccgctggg gaagctcatg gacccgggct ccctgccgcc cctcgactct
gaagatctct tccaggatct aagtcacttc caggagacgt ggctcgctga agctcaggta
ccagacagtg atgagcagtt tgttcctgat ttccattcag aaaacctagc tttccacagc
cccaccacca ggatcaagaa ggagccccag agtccccgca cagacccggc cctgtcctgc
agcaggaagc cgccactccc ctaccaccat ggcgagcagt gcctttactc cagtgcctat
gaccccccca gacaaatcgc catcaagtcc cctgcccctg gtgcccttgg acagtcgccc
ctacagccct ttccccgggc agagcaacgg aatttcctga gatcctctgg cacctcccag
ccccaccctg gccatgggta cctcggggaa catagctccg tcttccagca gcccctggac
atttgccact ccttcacatc tcagggaggg ggccgggaac ccctcccagc cccctaccaa
caccagctgt cggagccctg cccaccctat ccccagcaga gctttaagca agaataccat
gatcccctgt atgaacaggc gggccagcca gccgtggacc agggtggggt caatgggcac
aggtacccag gggcgggggt ggtgatcaaa caggaacaga cggacttcgc ctacgactca
gatgtcaccg ggtgcgcatc aatgtacctc cacacagagg gcttctctgg gccctctcca
ggctatggct atgagaaacc tctgcgacca ttcccagatg atgtctgcgt tgtccctgag
aaatttgaag gagacatcaa gcaggaaggg gtcggtgcat ttcgagaggg gccgccctac
cagcgccggg gtgccctgca gctgtggcaa tttctggtgg ccttgctgga tgacccaaca
aatgcccatt tcattgcctg gacgggccgg ggaatggagt tcaagctcat tgagcctgag
gaggtcgcca ggctctgggg catccagaag aaccggccag ccatgaatta cgacaagctg
agccgctcgc tccgatacta ttatgagaaa ggcatcatgc agaaggtggc tggtgagcgt
tacgtgtaca agtttgtgtg tgagcccgag gccctcttct ctttggcctt cccggacaat
cagcgtccag ctctcaaggc tgagtttgac cggcctgtca gtgaggagga cacagtccct
ttgtcccact tggatgagag ccccgcctac ctcccagagc tggctggccc cgcccagcca
tttggcccca agggtggcta ctcttactag cccccagcgg ctgttccccc tgccgcaggt
gggtgctgcc ctgtgtacat ataaatgaat ctggtgttgg ggaaaccttc atctgaaacc
cacagatgtc tctggggcag atccccactg tcctaccagt tgccctagcc cagactctga
gctgctcacc ggagtcattg ggaaggaaaa gtggagaaat ggcaagtcta gagtctcaga
aactcccctg ggggtttcac ctgggccctg gaggaattca gctcagcttc ttcctaggtc
caagcccccc acaccttttc cccaaccaca gagaacaaga gtttgttctg ttctggggga
cagagaaggc gcttcccaac ttcatactgg caggagggtg aggaggttca ctgagctccc
cagatctccc actgcgggga gacagaagcc tggactctgc cccacgctgt ggccctggag
ggtcccggtt tgtcagttct tggtgctctg tgttcccaga ggcaggcgga ggttgaagaa
aggaacctgg gatgaggggt gctgggtata agcagagagg gatgggttcc tgctccaagg
gaccctttgc ctttcttctg ccctttccta ggcccaggcc tgggtttgta cttccacctc
caccacatct gccagacctt aataaaggcc cccacttctc cca
ETV4 protein isoform 5
(SEQ ID NO: 20)
MERRMKAGYLDQQVPYTFSSKSPGNGSLREALIGPLGKLMDPGS
LPPLDSEDLFQDLSHFQETWLAEAQVPDSDEQFVPDFHSENLAFHSPTTRIKKEPQSP
RTDPALSCSRKPPLPYHHGEQCLYSSAYDPPRQIAIKSPAPGALGQSPLQPFPRAEQR
NFLRSSGTSQPHPGHGYLGEHSSVFQQPLDICHSFTSQGGGREPLPAPYQHQLSEPCP
PYPQQSFKQEYHDPLYEQAGQPAVDQGGVNGHRYPGAGVVIKQEQTDFAYDSDVTGCA
SMYLHTEGFSGPSPGYGYEKPLRPFPDDVCVVPEKFEGDIKQEGVGAFREGPPYQRRG
ALQLWQFLVALLDDPTNAHFIAWTGRGMEFKLIEPEEVARLWGIQKNRPAMNYDKLSR
SLRYYYEKGIMQKVAGERYVYKFVCEPEALFSLAFPDNQRPALKAEFDRPVSEEDTVP
LSHLDESPAYLPELAGPAQPFGPKGGYSY
MEOX2 RNA
(SEQ ID NO: 21)
gaaagcagtt ctctgggacc accttctttt ggcttcaacc tctcccactc ttgacatctg
agtagctcag ggaagctctt ccaggtccga ctgttcatat gtaaaggaga ctggccgctg
gggctcagga ccgggattat ccgagctctg cagaagtgca ccgctattgc tttgggaggt
taaaaaaaaa atcacacggt ttccagtgaa aaagtgacag agggtggtgg cctttggaac
cgccgtgaag tcttctgcct ggaacccgaa acttgcatgc tatggaacac ccgctctttg
gctgcctgcg cagccctcac gccacggcgc aaggcttgca cccgttctcc caatcctctc
tcgccctcca tggaagatct gaccatatgt cttaccccga gctctctact tcttcctcat
cttgcataat cgcgggatac cccaacgaag agggcatgtt tgccagccag catcacaggg
ggcaccacca ccaccaccac caccaccacc atcaccacca tcagcagcag cagcaccagg
ctctgcaaac caactggcac ctcccgcaga tgtcttcccc accgagtgcg gctcggcaca
gcctctgcct ccagcccgac tctggagggc ccccagagtt ggggagcagc ccgcccgtcc
tgtgctccaa ctcttccagc ttgggctcca gcaccccgac tggggccgcg tgcgcgccgg
gggactacgg ccgccaggca ctgtcacctg cggaggcgga gaagcgaagc ggcggcaaga
ggaaaagcga cagctcagac tcccaggaag gaaattacaa gtcagaagtc aacagcaaac
ccaggaaaga aaggacagca tttaccaaag agcaaatcag agaacttgaa gcagaatttg
cccatcataa ttatctcacc agactgaggc gatacgagat agcagtgaat ctggatctca
ctgaaagaca ggtgaaagtc tggttccaaa acaggcggat gaagtggaag agggtaaagg
gtggacagca aggagctgcg gctcgggaaa aggaactggt gaatgtgaaa aagggaacac
ttctcccatc agagctgtcg ggaattggtg cagccaccct ccagcaaaca ggggactcta
tagcaaatga agacagtcac gacagtgacc acagctcaga gcatgcgcac ttatgatata
aacagaggac cagctccatt ctcaggaaag aaatgttgtg atggcaagcc ttacccaaat
atcgtttaca cagagagatg actatggcag tgatgtttaa tattattaaa tccaggcatt
tcgaatctgt ttttcatgat ttatagaggg tttacacaaa gtgccactta ttaaagagct
tccacagtga agatggagaa ggtgaacttg ctttgaatat tccagatgtg tttggtcgtg
cgtatggcag tgagcaggta tgtgtttgct tttgcttgca ctgaaaatta aattgctatc
aagagcaaac tatgaacggt tttttattca agatgtctcc agagtgaaga tgccgaggat
gaacttgcat tgaacattcc agatgtgtga gatcatgtgt attacagtgg gcaggtattt
gcttttgctt gcactgaaaa ttaaattgct atcaagaata aaccatgaaa cattttatcc
tgaacagcca cagtgcctga attcactcaa gtggataaaa agtgtatttt aactctgtat
atattaccct taagtcattt tcctgtcttc actaatttag caatgcattc atattagctg
atgaaaatag gcactcacaa tgacaaccag agccagtttc ttgtcttttt tatacatttt
gtcatcccag agacaatcag tatgtgctta cctgtgttca agtagagaaa aatacagtag
agtctgatag gacatattct tgtaccacag acaaaacaaa tcttatgttg catttactat
caactgctgc taatacgtta ttataaaact tacctagctc ctgaattctt cctatcttat
agcttaaaac aattaggatc ataggcaaat cagttacctt gcagaaagag ctttgtatga
cagacattgt cttattttat ttctgtaaaa tattagctgt atgaatatga tttaattaac
aagaaaacat ttcttcctga ttgacaacag tgttagacaa ggtgcaaagc gaaactggtt
gctcaagttg atagaaaaca aaattctgaa tatcttcaaa ttaaattcgg taaaaacaca
ttattttttc atatgtgatg tattcatgca gaacaactat ctttgtattt tgtttttaaa
atgtgtttaa taaatgatcc tttgtaaata a
MEOX2 Protein
(SEQ ID NO: 22)
MEHPLFGCLRSPHATAQGLHPFSQSSLALHGRSDHMSYPELSTS
SSSCIIAGYPNEEGMFASQHHRGHHHHHHHHHHHHHQQQQHQALQTNWHLPQMSSPPS
AARHSLCLQPDSGGPPELGSSPPVLCSNSSSLGSSTPTGAACAPGDYGRQALSPAEAE
KRSGGKRKSDSSDSQEGNYKSEVNSKPRKERTAFTKEQIRELEAEFAHHNYLTRLRRY
EIAVNLDLTERQVKVWFQNRRMKWKRVKGGQQGAAAREKELVNVKKGTLLPSELSGIG
AATLQQTGDSIANEDSHDSDHSSEHAHL
PRKCB RNA isoform 1
(SEQ ID NO: 23)
ggacgagcgg cagcagctgg gcgagtgaca gccccggctc cgcgcgccgc ggccgccaga
gccggcgcag gggaagcgcc cgcggccccg ggtgcagcag cggccgccgc ctcccgcgcc
tccccggccc gcagcccgcg gtcccgcggc cccggggccg gcacctctcg ggctccggct
ccccgcgcgc aagatggctg acccggctgc ggggccgccg ccgagcgagg gcgaggagag
caccgtgcgc ttcgcccgca aaggcgccct ccggcagaag aacgtgcatg aggtcaagaa
ccacaaattc accgcccgct tcttcaagca gcccaccttc tgcagccact gcaccgactt
catctggggc ttcgggaagc agggattcca gtgccaagtt tgctgctttg tggtgcacaa
gcggtgccat gaatttgtca cattctcctg ccctggcgct gacaagggtc cagcctccga
tgacccccgc agcaaacaca agtttaagat ccacacgtac tccagcccca cgttttgtga
ccactgtggg tcactgctgt atggactcat ccaccagggg atgaaatgtg acacctgcat
gatgaatgtg cacaagcgct gcgtgatgaa tgttcccagc ctgtgtggca cggaccacac
ggagcgccgc ggccgcatct acatccaggc ccacatcgac agggacgtcc tcattgtcct
cgtaagagat gctaaaaacc ttgtacctat ggaccccaat ggcctgtcag atccctacgt
aaaactgaaa ctgattcccg atcccaaaag tgagagcaaa cagaagacca aaaccatcaa
atgctccctc aaccctgagt ggaatgagac atttagattt cagctgaaag aatcggacaa
agacagaaga ctgtcagtag agatttggga ttgggatttg accagcagga atgacttcat
gggatctttg tcctttggga tttctgaact tcagaaagcc agtgttgatg gctggtttaa
gttactgagc caggaggaag gcgagtactt caatgtgcct gtgccaccag aaggaagtga
ggccaatgaa gaactgcggc agaaatttga gagggccaag atcagtcagg gaaccaaggt
cccggaagaa aagacgacca acactgtctc caaatttgac aacaatggca acagagaccg
gatgaaactg accgatttta acttcctaat ggtgctgggg aaaggcagct ttggcaaggt
catgctttca gaacgaaaag gcacagatga gctctatgct gtgaagatcc tgaagaagga
cgttgtgatc caagatgatg acgtggagtg cactatggtg gagaagcggg tgttggccct
gcctgggaag ccgcccttcc tgacccagct ccactcctgc ttccagacca tggaccgcct
gtactttgtg atggagtacg tgaatggggg cgacctcatg tatcacatcc agcaagtcgg
ccggttcaag gagccccatg ctgtatttta cgctgcagaa attgccatcg gtctgttctt
cttacagagt aagggcatca tttaccgtga cctaaaactt gacaacgtga tgctcgattc
tgagggacac atcaagattg ccgattttgg catgtgtaag gaaaacatct gggatggggt
gacaaccaag acattctgtg gcactccaga ctacatcgcc cccgagataa ttgcttatca
gccctatggg aagtccgtgg attggtgggc atttggagtc ctgctgtatg aaatgttggc
tgggcaggca ccctttgaag gggaggatga agatgaactc ttccaatcca tcatggaaca
caacgtagcc tatcccaagt ctatgtccaa ggaagctgtg gccatctgca aagggctgat
gaccaaacac ccaggcaaac gtctgggttg tggacctgaa ggcgaacgtg atatcaaaga
gcatgcattt ttccggtata ttgattggga gaaacttgaa cgcaaagaga tccagccccc
ttataagcca aaagctagag acaagagaga cacctccaac ttcgacaaag agttcaccag
acagcctgtg gaactgaccc ccactgataa actcttcatc atgaacttgg accaaaatga
atttgctggc ttctcttata ctaacccaga gtttgtcatt aatgtgtagg tgaatgcaaa
ctccatcgtt gagcctgggg tgtaagactt caagccaagc gtatgtatca attctagtct
tccaggattc acggtgcaca tgctggcatt caacatgtgg aaagcttgtc ttagagggct
tttctttgta tgtgtagctt gctagtttgt tttctacatt tgaaaatgtt tagtttagaa
taagcgcatt atccaattat agaggtacaa ttttccaaac ttccagaaac tcatcaaatg
aacagacaat gtcaaaacta ctgtgtctga taccaaaatg cttcagtatt tgtaattttt
caagtcagaa gctgatgttc ctggtaaaag tttttacagt tattctataa tatcttcttt
gaatgctaag catgagcgat atttttaaaa attgtgagta agctttgcag ttactgtgaa
ctattgtctc ttggaggaag ttttttgttt aagaattgat atgattaaac tgaattaata
tatgcaa
PRKCB protein isoform 1
(SEQ ID NO: 24)
MADPAAGPPPSEGEESTVRFARKGALRQKNVHEVKNHKFTARFF
KQPIFCSHCIDFIWGFGKQGFQCQVCCFVVHKRCHEFVTFSCPGADKGPASDDPRSKH
KFKIHTYSSPTFCDHCGSLLYGLIHQGMKCDTCMMNVHKRCVMNVPSLCGTDHTERRG
RIYIQAHIDRDVLIVLVRDAKNLVPMDPNGLSDPYVKLKLIPDPKSESKQKIKTIKCS
LNPEWNETFRFQLKESDKDRRLSVEIWDWDLTSRNDFMGSLSFGISELQKASVDGWFK
LLSQEEGEYFNVPVPPEGSEANEELRQKFERAKISQGTKVPEEKTINTVSKFDNNGNR
DRMKLIDFNFLMVLGKGSFGKVMLSERKGIDELYAVKILKKDVVIQDDDVECTMVEKR
VLALPGKPPFLTQLHSCFQTMDRLYFVMEYVNGGDLMYHIQQVGRFKEPHAVFYAAEI
AIGLFFLQSKGIIYRDLKLDNVMLDSEGHIKIADFGMCKENIWDGVTIKTFCGTPDYI
APEIIAYQPYGKSVDWWAFGVLLYEMLAGQAPFEGEDEDELFQSIMEHNVAYPKSMSK
EAVAICKGLMTKHPGKRLGCGPEGERDIKEHAFFRYIDWEKLERKEIQPPYKPKARDK
RDTSNFDKEFTRQPVELTPTDKLFIMNLDQNEFAGFSYTNPEFVINV
PRKCB RNA isoform 2
(SEQ ID NO: 25)
ggacgagcgg cagcagctgg gcgagtgaca gccccggctc cgcgcgccgc ggccgccaga
gccggcgcag gggaagcgcc cgcggccccg ggtgcagcag cggccgccgc ctcccgcgcc
tccccggccc gcagcccgcg gtcccgcggc cccggggccg gcacctctcg ggctccggct
ccccgcgcgc aagatggctg acccggctgc ggggccgccg ccgagcgagg gcgaggagag
caccgtgcgc ttcgcccgca aaggcgccct ccggcagaag aacgtgcatg aggtcaagaa
ccacaaattc accgcccgct tcttcaagca gcccaccttc tgcagccact gcaccgactt
catctggggc ttcgggaagc agggattcca gtgccaagtt tgctgctttg tggtgcacaa
gcggtgccat gaatttgtca cattctcctg ccctggcgct gacaagggtc cagcctccga
tgacccccgc agcaaacaca agtttaagat ccacacgtac tccagcccca cgttttgtga
ccactgtggg tcactgctgt atggactcat ccaccagggg atgaaatgtg acacctgcat
gatgaatgtg cacaagcgct gcgtgatgaa tgttcccagc ctgtgtggca cggaccacac
ggagcgccgc ggccgcatct acatccaggc ccacatcgac agggacgtcc tcattgtcct
cgtaagagat gctaaaaacc ttgtacctat ggaccccaat ggcctgtcag atccctacgt
aaaactgaaa ctgattcccg atcccaaaag tgagagcaaa cagaagacca aaaccatcaa
atgctccctc aaccctgagt ggaatgagac atttagattt cagctgaaag aatcggacaa
agacagaaga ctgtcagtag agatttggga ttgggatttg accagcagga atgacttcat
gggatctttg tcctttggga tttctgaact tcagaaagcc agtgttgatg gctggtttaa
gttactgagc caggaggaag gcgagtactt caatgtgcct gtgccaccag aaggaagtga
ggccaatgaa gaactgcggc agaaatttga gagggccaag atcagtcagg gaaccaaggt
cccggaagaa aagacgacca acactgtctc caaatttgac aacaatggca acagagaccg
gatgaaactg accgatttta acttcctaat ggtgctgggg aaaggcagct ttggcaaggt
catgctttca gaacgaaaag gcacagatga gctctatgct gtgaagatcc tgaagaagga
cgttgtgatc caagatgatg acgtggagtg cactatggtg gagaagcggg tgttggccct
gcctgggaag ccgcccttcc tgacccagct ccactcctgc ttccagacca tggaccgcct
gtactttgtg atggagtacg tgaatggggg cgacctcatg tatcacatcc agcaagtcgg
ccggttcaag gagccccatg ctgtatttta cgctgcagaa attgccatcg gtctgttctt
cttacagagt aagggcatca tttaccgtga cctaaaactt gacaacgtga tgctcgattc
tgagggacac atcaagattg ccgattttgg catgtgtaag gaaaacatct gggatggggt
gacaaccaag acattctgtg gcactccaga ctacatcgcc cccgagataa ttgcttatca
gccctatggg aagtccgtgg attggtgggc atttggagtc ctgctgtatg aaatgttggc
tgggcaggca ccctttgaag gggaggatga agatgaactc ttccaatcca tcatggaaca
caacgtagcc tatcccaagt ctatgtccaa ggaagctgtg gccatctgca aagggctgat
gaccaaacac ccaggcaaac gtctgggttg tggacctgaa ggcgaacgtg atatcaaaga
gcatgcattt ttccggtata ttgattggga gaaacttgaa cgcaaagaga tccagccccc
ttataagcca aaagcttgtg ggcgaaatgc tgaaaacttc gaccgatttt tcacccgcca
tccaccagtc ctaacacctc ccgaccagga agtcatcagg aatattgacc aatcagaatt
cgaaggattt tcctttgtta actctgaatt tttaaaaccc gaagtcaaga gctaagtaga
tgtgtagatc tccgtccttc atttctgtca ttcaagctca acggctattg tggtgacatt
tttatgtttt tcattgccaa gttgcatcca tgtttgattt tctgatgaga ctagagtgac
agtgtttcag aacccaaatg tcctcaggta gtttggagca tctctatgag atgggattat
gcagatggcc tatggaaaat gcagctgcat aattaacaca ttatcaaagt cctcttacaa
tttattttcc gcagcatgtc agctaagtag acccaatggg gagagaaaat gcctgctttc
tttccctctt tttctgcact gccatattca cccccaacca tccaatctgt ggataattgg
atgttagcgg tactcttcca cttccgggcc tggagcttgg cttgtatcca agtgtatggt
tgctttgcct aagaggaatc cctctatttc acctgttctg gaggcaccag accttgaaaa
gaacatgctc aaaataaaat gttatctgtt atttttgtaa actcaaagtt aagatgatca
aagttctaaa attccaagaa tgtgctttta gacggtctca atctaaaagc acttcaaggg
gtcaaagggc aaccagcttg ggtgctacct cagtgttgta gtttctgata ctttatgtct
ttgctcaccc tcatccccaa actacttgaa aagggcattt ggcaccactc tctgaaacaa
cacagtcact ctagcaaggc ccccaaaggg ccctggtttt acattacatt tcaaacttta
tttgctttgg ggttttgttt ctgttgttgt tcaaatgcaa aaaaaagaaa aaaaaagaaa
aaaaaaggtg actcacattg ttacacatgc tttaaaatat gtattcaaat gttattaacc
acaatgacga cctgctttga tttaaccaag aagacggctg cggagcctag cagactcagg
cctgtgggaa tgggatttgt tacaaatcta ggtttgttac tggcttcaga aagctaatta
agtgctctga aaaagacacc gtttcttgaa acaaagatgg ttgtattcct cactttgatg
ttgttttgca agatgtttgt ggaaatgttc atttgtatct ggatctctgt tatgtgccat
ttttcttcta gcatcgagat acaataaaaa aaaaaaaaaa gaaaagaaga agaaatacta
tttcaaggaa aactgctctt tttgagaaac gtggacctaa actacaaagt gggaactgag
gagggaactc aggagaaagg aactaactgc ggagctttaa tcttggcccc agtgttcagc
cactcggagg ggcgggggct gtggcccatt caggggctgc tggtgggctg tagtggggtg
ggatgacctg gccagagcca acgaggatac tggagcccaa agtcaagttt agagaccagc
tgggaacgtg aatggggctc ttgattttct tatcaaaatc accactcctc ccagcttgga
ctaaatattc tttctagcaa gcagctttgt gagctccctg aagcccaagg aaacccttcg
gtgggagaaa tttcatttct gtctgagagg attaaggcag caggtgactc cccctcctcg
cctgccgtgt cctgctattc tcaggcagct ctaaggagaa ttcttatcac agttcaagtg
atttccagaa gttccagggc ttctgagaga ccatcaaggg aactttaaca acttgacaaa
tgtccttgaa gtaagatgcc tcatctttag ggaaaaatgg ggtttggatt tctgcttagg
caaagtctcc tgcagttcat ccttctctgt cctcttcttg cttcaggctt ggggaccgtc
cctgctgtcc ccactgtggt ggcaatcagg acctaaggtg aagcaaactt gaagttctat
ctgacaagtt taggcagtaa gagaaggagg gaaatcggag caaagctccc tcactttatt
gttgagaaac tggcatctgg aaagaggaag gaatttgccc aaagtcagtc agctgggata
aaaacctggg tgtcctgtcc agaaagtgca gggtgctttc tgctctgtag caaggcagca
gacatctctg agccaggccc accaacaggc ccttatctgg tggttggatc atgatcccat
tttgcttgga catgctctca ggaagataaa aaccatggag aaacactagg ccattgacaa
atgatctgag acaactttag aaaacaatgt aggatgaatg gaaagagaaa gaaaggaaag
aaagaagaaa aagaaagaag gaaagaaaga aagagaaagg aaggaaggaa agaaggaagg
aaaagaagga aggaaggaag gaatatagtg ttataaatac tgcactcaac attttccaaa
ttcttgccat tatttttcaa aagtttaata gtttgcagaa atagatactc aagccaaagt
ctgttttaga gaaactttcc atggaaagtc agaatttcta ccacttcctt ttctatccac
atttccagtg cagaagaaac tgagaaacag agctttttga agagaggaca gggccatagc
aacaaggacc ttcttggggg attaatggga ggtcagtaga attaataacc ctccttggat
gagtgctact gttttcacat ggcttcagat gctatcaacc tcaaagaaat gatctcaaca
gagaagctta ttctctccca acttctacgg taaaatccag gagtattttc tctggggatc
tgcccacagg acaaagtcca taaaagcaag tcctgtctgg accatgtggt tatctgaagc
attagccatc accagcacaa caaacggggc agggctttcc aaggtggggc tggtcagaag
ggaatctttg ataagaggcc cacaggcagg gaaagcgaaa tagggttgat gagaccaggg
gagacctaaa aaaaaggcag ctttgtgtct tctagctcca aatatacctg ccttttagct
cacacactgt cctggagttc tcagaccttt aggggcccta acacagttca gttcatacag
gggttcaaaa gggacagtgg cccatttggg agacctttag gatcaatggg aatcaattcc
attgttttgc ctcagagtaa agtttctggc tcggggacaa ttataagttg caaaaaggat
agaggcatat cccaagtctt ccttcattcc acaaataatt acaaacaacc tactgtgtgc
caggcactat tcttagcact ggaaatacac tagtgaagaa gcagatgagg accctgttta
ttgtttctct ccaagaaatt ctccaagaat attgtttctt ggagagaaat aataaataaa
caagacaatt tctgaaagca ataagtgcaa tcaagataat taaaggatgc taaagtgtga
cttgtgggga ttgggagaga gatgcacaga caatattaaa gaggaggcat tcgagctttg
ttgtgaacac cggaagtaac atgccgagcg cctgggggat ggaaactcct atagcacccc
acaggctaac agcaagcagg acaagacaaa aagggcaggt gggacatggt agagatggac
cctacccagg aaacagctcc atcagcatct tagcctgccc cactctagcc acacataccc
acgtgtgctc ctgagttcag tgtgcccacc tcactcccac accctcacat agacttggca
agagtaagga gggaactcca tagagacatt ttacctatct caggggagca gccacaaaga
agcaagtctt gtaaaaggtc ttttgcaaag gagagtgaac ccagcaatga gagatcctta
acagctagtg cccattaggg ggctaaacct aaagcctggg tggtgatggc tcaaacgcta
atgagtcagt gaatccttac cgaccccctg gcctttataa tctgaggcaa ctttggctgc
agcccgggaa tgtgcagggc actagggaat acaaggcctt cttccctggt tgtcttgtaa
taaaacagcc atggggttgt ccctccagtc cgagagactg tgatgaggcc tacatagcag
cgatgtggtc aggtaaaaat caggaaccca ctgaaatctt gggcaagcca ccctgcctgc
ttgtgcctcg gttctctcat atgtcatata taggaggtga ggactccagc tccacctgcc
ccaggtgggt gtggtgatga tgaggaaaga caagaggctt gcaaggaccc tgaagaggtc
ggagcatcat acagattcct ttattagccc acattctgat gttccctggt gagacttgcc
ccaagcaatt gctagtaaat gggggttaat ttcttctcca cctccctact gaacaaaaaa
agaaatgcca gacttactag gagaatcgag ttgctttgag tttcttttgt tttgttttgt
tttgttttgt tttaaggctc cccttacaca ccctccttta agctttgggt tttctctctt
atagtttgtt gacacatgct aaaaatgtct ttggagagaa cttctgcctg ataaacaccc
aattctagac tgtgggtgga ttttcgagct gacggtggtc aattcctttc attaagcagt
gatctgattt ctccacatgg ccattctgcc ttcttggggg cagagtagat gggcagcagt
tcaccttttc agagaaagag gtcttctagc cacctgggct gctactgaat ggttttctcc
aggacgctct acctaatgat tatttctata acattaagca tggtaataag tagcttccaa
ttcaattcat cctaaagcca aagaaaatac agcaacacac acacacacac acacacacac
acacacacac acacacacac accactttat ggcaattctt aactgacatt caatgactta
cttcttttct tagaaaattt ccaccacatt tctatcccca agccaacata caatgtgaaa
tgaaagccag tgcgtggagt gcagctgcta aaaattttca gcacagggct ctttctgact
ctgctcatga gatggtatca gccacccaat gactggcgta tcttggtcct gtgtctttct
tcttacgctg tgttaatgtg tttactttcc atttggcaga gagacaagag agacacctcc
aacttcgaca aagagttcac cagacagcct gtggaactga cccccactga taaactcttc
atcatgaact tggaccaaaa tgaatttgct ggcttctctt atactaaccc agagtttgtc
attaatgtgt aggtgaatgc aaactccatc gttgagcctg gggtgtaaga cttcaagcca
agcgtatgta tcaattctag tcttccagga ttcacggtgc acatgctggc attcaacatg
tggaaagctt gtcttagagg gcttttcttt gtatgtgtag cttgctagtt tgttttctac
atttgaaaat gtttagttta gaataagcgc attatccaat tatagaggta caattttcca
aacttccaga aactcatcaa atgaacagac aatgtcaaaa ctactgtgtc tgataccaaa
atgcttcagt atttgtaatt tttcaagtca gaagctgatg ttcctggtaa aagtttttac
agttattcta taatatcttc tttgaatgct aagcatgagc gatattttta aaaattgtga
gtaagctttg cagttactgt gaactattgt ctcttggagg aagttttttg tttaagaatt
gatatgatta aactgaatta atatatgcaa
PRKCB Protein isoform 2
(SEQ ID NO: 26)
MADPAAGPPPSEGEESTVRFARKGALRQKNVHEVKNHKFTARFF
KQPIFCSHCIDFIWGFGKQGFQCQVCCFVVHKRCHEFVTFSCPGADKGPASDDPRSKH
KFKIHTYSSPTFCDHCGSLLYGLIHQGMKCDTCMMNVHKRCVMNVPSLCGTDHTERRG
RIYIQAHIDRDVLIVLVRDAKNLVPMDPNGLSDPYVKLKLIPDPKSESKQKIKTIKCS
LNPEWNETFRFQLKESDKDRRLSVEIWDWDLTSRNDFMGSLSFGISELQKASVDGWFK
LLSQEEGEYFNVPVPPEGSEANEELRQKFERAKISQGTKVPEEKTINTVSKFDNNGNR
DRMKLIDFNFLMVLGKGSFGKVMLSERKGIDELYAVKILKKDVVIQDDDVECTMVEKR
VLALPGKPPFLTQLHSCFQTMDRLYFVMEYVNGGDLMYHIQQVGRFKEPHAVFYAAEI
AIGLFFLQSKGIIYRDLKLDNVMLDSEGHIKIADFGMCKENIWDGVTIKTFCGTPDYI
APEIIAYQPYGKSVDWWAFGVLLYEMLAGQAPFEGEDEDELFQSIMEHNVAYPKSMSK
EAVAICKGLMTKHPGKRLGCGPEGERDIKEHAFFRYIDWEKLERKEIQPPYKPKACGR
NAENFDRFFTRHPPVLIPPDQEVIRNIDQSEFEGFSFVNSEFLKPEVKS
DDN RNA
(SEQ ID NO: 27)
ggctctgcag tgggcgccgg ctccctgggc tgggaggggg ctcctggggc gggtgggagg
gtggggggcc ggggtggggt ggggcaggat gctggatggc ccactgttct ccgaggggcc
tgacagcccc cgggagctcc aggatgagga gtctggcagc tgcctctggg tgcagaagtc
caagctattg gtgatagaag tgaagactat ttcctgtcat tatagtcgcc gcgccccttc
tcgacagccc atggacttcc aggccagcca ctgggctcgc gggttccaga accgcacgtg
tgggccgcgc ccgggatccc cacagccgcc gccccgccgg ccctgggcct ccagggtgct
gcaggaggcg accaactggc gggcggggcc cctggccgag gtccgagctc gggagcaaga
gaaaaggaaa gcggcgtcgc aggagcggga ggccaaggag accgagcgaa aaaggcgcaa
ggctggtggg gcccgacgga gccccccggg tcgaccccgc ccggagcccc gcaacgcccc
tcgggtggcc cagctggcag ggctccctgc tcccttgcgg ccggagcgcc tggcgcctgt
ggggcgagcg ccccgtccat ccgcgcagcc gcagagcgac ccagggtcgg cgtgggcggg
gccctgggga ggtcggcggc ccgggccccc aagctacgag gctcacctgc tgctgagagg
ttctgccggg accgccccac gacgccgctg ggaccggccg ccaccctacg tggctccacc
ttcttacgaa ggcccccata ggaccttggg gactaagaga ggccccggga actctcaggt
gcccacttca tcagccccag ctgcgactcc agccaggaca gacggagggc gcacaaagaa
gaggctggat cctcggatct accgggacgt cctcggggct tggggtctcc gacaggggca
aggtctcttg gggggatccc caggctgtgg agcggccaga gcaaggccag agcccggcaa
gggggtcgtg gagaaaagcc tggggctggc tgctgctgac ctgaacagtg gtagcgacag
ccatccccaa gccaaagcta cagggagcgc aggcaccgag atagctcctg cggggtctgc
aactgcggct ccctgtgccc cgcatcccgc tcccagatcc aggcaccacc tcaagggctc
gagggaaggg aaagaaggag aacagatctg gtttcccaaa tgctggattc cctcccctaa
aaagcagccg ccccgccata gccagacact ccccagaccc tgggctcccg gaggcaccgg
atggagagaa tctctgggtc ttggagaggg ggcaggaccg gagaccctgg agggttggaa
ggcgacccgc cgtgcccaca ccttgccccg cagttcccag ggcctgtccc gtggggaagg
cgtctttgtc attgacgcca cgtgcgtagt gatacgatcc caatatgttc caaccccccg
aacccagcag gtgcagcttt tgccctctgg ggtgacacgc gtggtggggg attcccccag
ccaatcgaag cccggcaagg aggagggtga aggggccacg gtctttcctt ccccttgtca
aaagcggctg tcgagcagtc gccttttaca ccagcccggc gggggccgcg ggggcgaagc
tgagggcggg aggccggggg actccacact ggaggagcgc actttccgca tcttggggct
cccggccccc gaagtaaacc tgcgggacgc ccccacgcag ccaggtagcc cagagcacca
agccttaggc ccagcagctt cgggagccca gggcagagcc gaggggtcgg aagtggcggt
ggtccagcgg cgcgccggcc ggggctgggc gcggacccca gggccctacg ccggggccct
gcgagaagcc gtgtcccgta tccgccgcca cacagcccct gactcggaca cggacgaagc
tgaggagctc agcgtccata gcggctcctc tgatggaagc gacacagaag ccccgggcgc
ctcctggcgg aatgagagga ccctgcccga ggttggaaac agttcgccag aggaagatgg
gaagacagcg gaactgagcg acagtgtcgg ggagatccta gatgtcataa gccaaaccga
ggaggtcctc ttcggggtga gggacatcag agggacccaa cagggaaata ggaagaggca
gtgagaggcc ccttcttgta tttgtgtccc caacgcatcc atccttgggt ccactggtcc
ccattcttcc ccacagactt cctttgcttc tcttttcctt gtatctttac ccatacctgt
tctcatcctt gaaatataaa tgaaaggaag ggaagcatat gcccattaat gattttgttt
caggagaggt gagaatgagc agatttaatt aatgtctgtt atgttcaggg cacaagggtg
agctcttcgc aggggctgat gcactgggtg tggagctgag cagagaggcc taaccaggat
caggcaggag ggcagggatg gtggcagcca taggagggca gggtagggta gggcctctga
ggaggaggga aaaagtgaag gagaggcttt ggacctggtg acagagtgat cagatgacag
aggggttctt gggagaagag gcataggtcc agcaacaacc aacaaagcag aaggagggct
caccttggtg tcacaagtct tggatttcaa tcccaactct gccactgagt tgctggttga
ctgaggccag tcactttccc tctccaggcc tccaggcctc ctggtatata aaatgatggt
attctaaggt ccatccttcc gtctctgaca ttttgagatc tttggaaagg actctatctc
atcctcccct cgacaagcca agaatgagaa ttgggaataa gtgaacagag tttgagggtt
tctgggcggc ctccgtgtca cccaaagtca tgatcaattc aggagactgc ccaaggcttg
cagaagaggt aagggagtga ggcactccta tcccagtctc ccaggtttgg ttgagggctc
cccaaggcag ggcaagatag cggccctgtc actgaccctg gcctgtggtg gtctgagctg
gggagggaag gacaccaatg aatcagcttg ggacctcttt aggccttccc cttttcctcc
accccgatgc tccttagtga tgctctgagg cgtggccacg atctccctcc caggtggtat
cgcccacctg aaaaaatcct gagaatttct cccatcttgg cctcttccag aaaccggcca
ggcaaggaaa gaggccggtc accagaagcc agcaggcgtg gggtgtgata ctctctatag
ccactacagg gcgcgcgcag gtcgcggatc tccccagttg ctaatcccgg ctctgccact
caatcctatc cctagttccc gagcgcgggt cccccgcctt gcagtctcca gccgtgcggg
gccgggagca ggcctccggc ctcccagact tctagagccc gccgggccca tctttgtact
catccacccc agccggcttg ggactcagac accgaagtct tttttttttt ctctccgatc
cttggacacc tcctctgtct gccatttatt agccatgtga acttggccac atcacttcac
ctccctgagc ctcagtttcc tcatctgtca aatgggggtt tataaacacc tacctcgcag
ggttgttgtg aggatttaat gcgataatgt atgtaaagcg ccttgcacac tgcctggcac
acagtaggcg ctcaataaat ctaagcttcc cttta
DDN Protein
(SEQ ID NO: 28)
MLDGPLFSEGPDSPRELQDEESGSCLWVQKSKLLVIEVKTISCH
YSRRAPSRQPMDFQASHWARGFQNRICGPRPGSPQPPPRRPWASRVLQEATNWRAGPL
AEVRAREQEKRKAASQEREAKETERKRRKAGGARRSPPGRPRPEPRNAPRVAQLAGLP
APLRPERLAPVGRAPRPSAQPQSDPGSAWAGPWGGRRPGPPSYEAHLLLRGSAGTAPR
RRWDRPPPYVAPPSYEGPHRTLGTKRGPGNSQVPTSSAPAATPARTDGGRTKKRLDPR
IYRDVLGAWGLRQGQGLLGGSPGCGAARARPEPGKGVVEKSLGLAAADLNSGSDSHPQ
AKATGSAGTEIAPAGSATAAPCAPHPAPRSRHHLKGSREGKEGEQIWFPKCWIPSPKK
QPPRHSQTLPRPWAPGGTGWRESLGLGEGAGPETLEGWKATRRAHTLPRSSQGLSRGE
GVFVIDATCVVIRSQYVPTPRTQQVQLLPSGVTRVVGDSPSQSKPGKEEGEGATVFPS
PCQKRLSSSRLLHQPGGGRGGEAEGGRPGDSTLEERTFRILGLPAPEVNLRDAPTQPG
SPEHQALGPAASGAQGRAEGSEVAVVQRRAGRGWARTPGPYAGALREAVSRIRRHTAP
DSDTDEAEELSVHSGSSDGSDTEAPGASWRNERTLPEVGNSSPEEDGKTAELSDSVGE
ILDVISQTEEVLFGVRDIRGTQQGNRKR
OTP mRNA
(SEQ ID NO: 29)
attataatgc aagaagcccc ctttttaacc acaaaccgaa ttttctttca tttaggtgat
ctatatatat ctatatcgta tagcttatag cttatatcta ttttaaataa cttaaagccg
ctaaaatttg ggggggaaca gctttcgccc tggagcggtg cgcgatgctg tctcatgccg
acctcctgga cgccaggcta ggtatgaaag atgccgccga gcttctgggc caccgggagg
cggtgaagtg taggctgggc gtggggggct ccgaccccgg gggccatccg ggggacctgg
cgcccaactc tgacccagtg gagggagcca ctctgctgcc cggggaggac atcaccacag
tgggctctac tccggcctcg ctggcggtga gcgccaaaga cccggacaag cagcccgggc
cccagggcgg cccgaacccc agccaagccg gccagcagca gggccaacag aagcagaagc
gccaccggac gcgcttcacc cccgcacagc tcaacgagtt ggagaggagc ttcgccaaga
ctcactaccc cgacatcttt atgcgtgagg agctggcact gcgtatcggg ctgaccgagt
cccgagtgca ggtctggttc cagaaccgac gcgccaagtg gaagaagcgc aaaaagacga
ccaacgtgtt ccgtgcgccc ggcacactgc tgcccacgcc aggcctgcct cagttcccgt
cggctgccgc cgccgctgcc gccgccatgg gcgacagcct gtgctctttc cacgccaacg
acacccgctg ggcggcggcc gccatgcctg gcgtgtcaca gctgcctctg ccgccggcgc
tgggcaggca gcaggccatg gcgcagtcgc tgtcccagtg cagcctggcg gccggtccgc
cgcccaactc catgggcctg tccaacagcc tggcgggttc caacggcgcg gggctgcagt
cgcacctcta ccagcccgcc ttccccggca tggtgcccgc ctccctcccc ggccccagca
acgtctccgg ttcgccccag ctctgcagct ccccggacag cagcgacgtg tggcggggca
ccagcatcgc ctccctccgc cgcaaggcgc tagagcacac agtctctatg agcttcactt
aatgcagccg cgccccggcc cgctccgccc ccagcaccgc cccgggggcc gccccgaggc
ccttccggcg cgcacccgga ccccggcgcc ctgccccgtc ccgccccggc cttcgccccg
tctcgtttcg tcctcgcctc tctcctccac tcgctcgggc tcaccccaag ccccagcccg
cgaggcctcc cctccgcctg atttcgatcg cccgcggtcc cccgtctccc ggccgcccct
cttcccttcc cacccagctg cgccctcggc tcggtctcca gcgcctcagc ccacccttcc
cgccaccctg gcctccctgc ttgcgctggc cgtgctcgcg ccctcctcct ggccttctga
cgggcggcgt tcccacccac accttcgacg cgacgcctac gacccccctc gcccgccgcc
tcccctccgg tcccctcttt ccccacactt cgcgaccctc ctcccgcgcc cggcaaaaag
tatccttccc gccattttac gtaccaggga gtcgactcag gatctgaaat cagacaccaa
tggactggtt tgtgggcaga aacacacaca ctcgcactct cgctcacgct cagacgctac
acacgcgcgc gcacagacac ggtgcaccta ggtcacacac ggacgtgttc aagggacagc
acaatgttag ggatttttgt cttaaaggag gacaagcatt gctaccaacc gcctcatctg
agggcccaac tgatatgatt tgatttatcc ttgtactctc caagctcctg tctttctttc
ctctcccacc acgctaccct tgcccagtcc acccagtcac atccgtgcag ccctctcttg
gcttgcaaga taacgctttt atttttattt tatcttattt tcattttctt aagcacaact
gtgtgagagt gtagaaggga aggcttctca ggaggaacgt gacagtggat tgggtggctg
gagtagacta aagcagtcat gtgacgagga agaggtgatc tgacccattt tgataagtct
ttataaggaa gaataaaata aacgtgtaag caaaattttc ttttgtaaaa gcaaaagcca
catctctttt ctggatcctt caggactggg gtttgtttgc ttccttttct gtttctgtct
tctcgctgct ctgtgccctt ggttgttttg tggtggtcct gtcgtccctc gtgcccctcg
gccacctgct ggcagccgat gggggcactc ggacatctac aaccctgcaa ctttgtacag
agaaacacaa tcagctcttt ctgcatgtgc tggtcaaatc caaacccaga gaacagaagc
gctttctaag aatgaacaaa tatgtgaaat aggatgtttt gtgtagataa agcattcttg
ttacatactg gtcaatttgt gatatgtttt aacttaatgt ctgtgtttat ttatggaatt
cggttttctt aataaatgtt tgagctaata taaagcatat tatttgactt ttccggacaa
gtttatatca agttaaatgt aaatggataa aataaaatca ttttcagtat gtga
OTP Protein
(SEQ ID NO: 30)
MLSHADLLDARLGMKDAAELLGHREAVKCRLGVGGSDPGGHPGD
LAPNSDPVEGATLLPGEDITTVGSTPASLAVSAKDPDKQPGPQGGPNPSQAGQQQGQQ
KQKRHRTRFTPAQLNELERSFAKTHYPDIFMREELALRIGLTESRVQVWFQNRRAKWK
KRKKTTNVFRAPGTLLPTPGLPQFPSAAAAAAAAMGDSLCSFHANDTRWAAAAMPGVS
QLPLPPALGRQQAMAQSLSQCSLAAGPPPNSMGLSNSLAGSNGAGLQSHLYQPAFPGM
VPASLPGPSNVSGSPQLCSSPDSSDVWRGTSIASLRRKALEHTVSMSFT

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method of reprogramming an astrocyte to a glioblastoma stem-like cell (GSC) by introducing at least one master regulator selected from the group consisting of: NKX2-2, ETV4, MLXIPL, MEOX2, PRKCB, DDN, or OTP into a cell.

2. The method of claim 1, wherein the master regulator is MEOX2, PRKCB, or ETV4.

3. The method of claim 1, wherein the master regulator is MEOX2, and further comprises introducing NKX6-2.

4. The method of claim 1, wherein the master regulators are MEOX2 and PRKCB, MEOX2 and ETV4.

5. The method of claim 1, wherein the master regulators are NKX2-2, ETV4, MLXIPL, MEOX2, PRKCB, DDN, and OTP.

6. The method of claim 1, further comprising introducing at least one master regulator selected from the group consisting of: BASP1, NKX6.2, STOX2, MYCN, SOX8, OLIG2, HES6, and ASCL1.

7. A method of inhibiting a glioblastoma stem-like cell (GSC) by administering an immunotherapy composition that inhibits or reduces the expression of at least one master regulator selected from the group consisting of: NKX2-2, ETV4, MLXIPL, MEOX2, PRKCB, DDN, or OTP.

8. A method of treating a subject for glioblastoma by administering an immunotherapy composition that inhibits or reduces the expression of at least one master regulator selected from the group consisting of: NKX2-2, ETV4, MLXIPL, MEOX2, PRKCB, DDN, or OTP.

9. The method of any one of claims 7-8, wherein the master regulator is MEOX2, PRKCB, or ETV4.

10. The method of any one of claims 7-8, wherein the immunotherapy composition targets at least two master regulators.

11. The method of claim 10, wherein the master regulators are MEOX2 and PRKCB or MEOX2 and ETV4.

12. The method of claim 9, wherein the master regulator is MEOX2 and the immunotherapy composition further comprises an inhibitor of NKX6-2.

13. The method of any one of claim 10, wherein the immunotherapy composition targets NKX2-2, ETV4, MLXIPL, MEOX2, PRKCB, DDN, and OTP.

14. The method of any one of claims 7-8, wherein the immunotherapy composition comprises a peptide formulation derived from at least one master regulator, nanoparticles containing peptides derived from at least one master regulator, dendritic cells containing peptides derived from at least one master regulator, RNA coding at least one master regulator, nanoparticles containing RNA coding at least one master regulator, or dendritic cells containing RNA coding at least one master regulator.

15. The method of any one of claims 7-8, wherein, the inhibitor is a RNA interference agent or a small molecule.

16. An immunotherapy composition for treating a subject with a glioblastoma, comprising an inhibitor of at least one of NKX2-2, ETV4, MLXIPL, MEOX2, PRKCB, DDN, or OTP.

17. The immunotherapy composition of claim 16, wherein the composition comprises an inhibitor of MEOX2, PRKCB, or ETV4.

18. The immunotherapy composition of claim 17, wherein the composition comprises an inhibitor of MEOX2, and further comprises an inhibitor of NKX6-2.

19. The immunotherapy composition of claim 18, wherein the composition comprises inhibitors of MEOX2 and PRKCB, MEOX2 and ETV4, or MEOX2 and NKX6-2.

20. The immunotherapy composition of claim 18, where the composition comprises inhibitors of NKX2-2, ETV4, MLXIPL, MEOX2, PRKCB, DDN, and OTP.

21. A kit, comprising a first container and a second container, wherein the first container comprises at least one dose of a composition comprising an inhibitor of at least one master regulator selected from the group consisting of: NKX2-2, ETV4, MLXIPL, MEOX2, PRKCB, DDN, or OTP.


SEQUENCE LISTING (30 sequences)

SEQ ID NO: 1 - DNA, 2123 bp, Homo sapiens
ccattttttcctcgccaccagccgccaccgcgcgccgagcggccgccggagcccgagctgacgccgccttggcacccctcctggagttagaaactaaggccggggcccgcggcgctcggcgcgcaggccgcccggcttcctgcgtccatttccgcgtgctttcaaagaagacagagagaggcactgggttgggcttcatttttttcctccccatccccagtttctttctctttttaaaaataataattatcccaataattaaagccaattcccccctcccctcccccagtccctccccccaactcccccctcccccgcccgccggggcaggggagcgccacgaattgaccaagtgaagctacaactttgcgacataaattttggggtctcgaaccatgtcgctgaccaacacaaagacggggttttcggtcaaggacatcttagacctgccggacaccaacgatgaggagggctctgtggccgaaggtccggaggaagagaacgaggggcccgagccagccaagagggccgggccgctggggcagggcgccctggacgcggtgcagagcctgcccctgaagaaccccttctacgacagcagcgacaacccgtacacgcgctggctggccagcaccgagggccttcagtactccctgcacggtctggctgccggggcgccccctcaggactcaagctccaagtccccggagccctcggccgacgagtcaccggacaatgacaaggagaccccgggcggcgggggggacgccggcaagaagcgaaagcggcgagtgcttttctccaaggcgcagacctacgagctggagcggcgctttcggcagcagcggtacctgtcggcgcccgagcgcgaacacctggccagcctcatccgcctcacgcccacgcaggtcaagatctggttccagaaccaccgctacaagatgaagcgcgcccgggccgagaaaggtatggaggtgacgcccctgccctcgccgcgccgggtggccgtgcccgtcttggtcagggacggcaaaccatgtcacgcgctcaaagcccaggacctggcagccgccaccttccaggcgggcattcccttttctgcctacagcgcgcagtcgctgcagcacatgcagtacaacgcccagtacagctcggccagcaccccccagtacccgacagcacaccccctggtccaggcccagcagtggacttggtgagcgccgccccaacgagactcgcggccccaggcccaggccccaccccggcggcggtggcggcgaggaggcctcggtccttatggtggttattattattattataattattattatggagtcgagttgactctcggctccactagggaggcgccgggaggttgcctgcgtctccttggagtggcagattccacccacccagctctgcccatgcctctccttctgaaccttgggagagggctgaactctacgccgtgtttacagaatgtttgcgcagcttcgcttctttgcctctccccggggggaccaaaccgtcccagcgttaatgtcgtcacttgaaaacgagaaaaagaccgaccccccacccctgctttcgtgcattttgtaaaatatgtttgtgtgagtagcgatattgtcagccgtcttctaaagcaagtggagaacactttaaaaatacagagaatttcttcctttttttaaaaaaaaataagaaaatgctaaatatttatggccatgtaaacgttctgacaactggtggcagatttcgcttttcgttgtaaatatcggtggtgattgttgccaaaatgaccttcaggaccggcctgtttcccgtctgggtccaactcctttctttgtggcttgtttgggtttgttttttgttttgtttttgtttttgcgttttcccctgctttcttcctttctctttttattttattgtgcaaacatttctcaaatatggaaaagaaaaccctgtaggcagggagccctctgccctgtcctccgggccttcagccccgaacttggagctcagctattcggcgcggttccccaacagcgccgggcgcagaaagctttcgattttttaaataagaattttaataaaaatcctgtgtttaaaaaagaaaaaaa

SEQ ID NO: 2 - protein, 273 aa, Homo sapiens
MSLTNTKTGFSVKDILDLPDTNDEEGSVAEGPEEENEGPEPAKRAGPLGQGALDAVQSLPLKNPFYDSSDNPYTRWLASTEGLQYSLHGLAAGAPPQDSSSKSPEPSADESPDNDKETPGGGGDAGKKRKRRVLFSKAQTYELERRFRQQRYLSAPEREHLASLIRLTPTQVKIWFQNHRYKMKRARAEKGMEVTPLPSPRRVAVPVLVRDGKPCHALKAQDLAAATFQAGIPFSAYSAQSLQHMQYNAQYSSASTPQYPTAHPLVQAQQWTW

SEQ ID NO: 3 - DNA, 3252 bp, Homo sapiens
agggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactcagtgcccagcccagcctcaaggtgagcaaagctaccacgctgcagaagacagctgagtacatccttatgctacagcaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttctctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtc

SEQ ID NO: 4 - protein, 852 aa, Homo sapiens
MAGALAGLAAGLQVPRVAPSPDSDSDTDSEDPSLRRSAGGLLRSQVIHSGHFMVSSPHSDSLPRRRDQEGSVGPSDFGPRSIDPTLTRLFECLSLAYSGKLVSPKWKNFKGLKLLCRDKIRLNNAIWRAWYIQYVKRRKSPVCGFVTPLQGPEADAHRKPEAVVLEGNYWKRRIEVVMREYHKWRIYYKKRLRKPSREDDLLAPKQAEGRWPPPEQWCKQLFSSVVPVLLGDPEEEPGGRQLLDLNCFLSDISDTLFTMTQSGPSPLQLPPEDAYVGNADMIQPDLTPLQPSLDDFMDISDFFTNSRLPQPPMPSNFPEPPSFSPVVDSLFSSGTLGPEVPPASSAMTHLSGHSRLQARNSCPGPLDSSAFLSSDFLLPEDPKPRLPPPPVPPPLLHYPPPAKVPGLEPCPPPPFPPMAPPTALLQEEPLFSPRFPFPTVPPAPGVSPLPAPAAFPPTPQSVPSPAPTPFPIELLPLGYSEPAFGPCFSMPRGKPPAPSPRGQKASPPTLAPATASPPTTAGSNNPCLTQLLTAAKPEQALEPPLVSSTLLRSPGSPQETVPEFPCTFLPPTPAPTPPRPPPGPATLAPSRPLLVPKAERLSPPAPSGSERRLSGDLSSMPGPGTLSVRVSPPQPILSRGRPDSNKTENRRITHISAEQKRRFNIKLGFDTLHGLVSTLSAQPSLKVSKATTLQKTAEYILMLQQERAGLQEEAQQLRDEIEELNAAINLCQQQLPATGVPITHQRFDQMRDMFDDYVRTRTLHNWKFWVFSILIRPLFESFNGMVSTASVHTLRQTSLAWLDQYCSLPALRPTVLNSLRQLGTSTSILTDPGRIPEQATRAVTEGTLGKPL

SEQ ID NO: 5 - DNA, 3195 bp, Homo sapiens
agggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaccgagaaccggcgtatcacacacatctccgcggagcagaagcggcgcttcaacatcaagctggggtttgacacccttcatgggctcgtgagcacactcagtgcccagcccagcctcaaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttctctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtc

SEQ ID NO: 6 - protein, 833 aa, Homo sapiens
MAGALAGLAAGLQVPRVAPSPDSDSDTDSEDPSLRRSAGGLLRSQVIHSGHFMVSSPHSDSLPRRRDQEGSVGPSDFGPRSIDPTLTRLFECLSLAYSGKLVSPKWKNFKGLKLLCRDKIRLNNAIWRAWYIQYVKRRKSPVCGFVTPLQGPEADAHRKPEAVVLEGNYWKRRIEVVMREYHKWRIYYKKRLRKPSREDDLLAPKQAEGRWPPPEQWCKQLFSSVVPVLLGDPEEEPGGRQLLDLNCFLSDISDTLFTMTQSGPSPLQLPPEDAYVGNADMIQPDLTPLQPSLDDFMDISDFFTNSRLPQPPMPSNFPEPPSFSPVVDSLFSSGTLGPEVPPASSAMTHLSGHSRLQARNSCPGPLDSSAFLSSDFLLPEDPKPRLPPPPVPPPLLHYPPPAKVPGLEPCPPPPFPPMAPPTALLQEEPLFSPRFPFPTVPPAPGVSPLPAPAAFPPTPQSVPSPAPTPFPIELLPLGYSEPAFGPCFSMPRGKPPAPSPRGQKASPPTLAPATASPPTTAGSNNPCLTQLLTAAKPEQALEPPLVSSTLLRSPGSPQETVPEFPCTFLPPTPAPTPPRPPPGPATLAPSRPLLVPKAERLSPPAPSGSERRLSGDLSSMPGPGTLSVRVSPPQPILSRGRPDSNKTENRRITHISAEQKRRFNIKLGFDTLHGLVSTLSAQPSLKERAGLQEEAQQLRDEIEELNAAINLCQQQLPATGVPITHQRFDQMRDMFDDYVRTRTLHNWKFWVFSILIRPLFESFNGMVSTASVHTLRQTSLAWLDQYCSLPALRPTVLNSLRQLGTSTSILTDPGRIPEQATRAVTEGTLGKPL

SEQ ID NO: 7 - DNA, 3246 bp, Homo sapiens
agggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaaccggcgtatcacacacatctccgcggagcagaagcggcgcttcaacatcaagctggggtttgacacccttcatgggctcgtgagcacactcagtgcccagcccagcctcaaggtgagcaaagctaccacgctgcagaagacagctgagtacatccttatgctacagcaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttctctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtc

SEQ ID NO: 8 - protein, 850 aa, Homo sapiens
MAGALAGLAAGLQVPRVAPSPDSDSDTDSEDPSLRRSAGGLLRSQVIHSGHFMVSSPHSDSLPRRRDQEGSVGPSDFGPRSIDPTLTRLFECLSLAYSGKLVSPKWKNFKGLKLLCRDKIRLNNAIWRAWYIQYVKRRKSPVCGFVTPLQGPEADAHRKPEAVVLEGNYWKRRIEVVMREYHKWRIYYKKRLRKPSREDDLLAPKQAEGRWPPPEQWCKQLFSSVVPVLLGDPEEEPGGRQLLDLNCFLSDISDTLFTMTQSGPSPLQLPPEDAYVGNADMIQPDLTPLQPSLDDFMDISDFFTNSRLPQPPMPSNFPEPPSFSPVVDSLFSSGTLGPEVPPASSAMTHLSGHSRLQARNSCPGPLDSSAFLSSDFLLPEDPKPRLPPPPVPPPLLHYPPPAKVPGLEPCPPPPFPPMAPPTALLQEEPLFSPRFPFPTVPPAPGVSPLPAPAAFPPTPQSVPSPAPTPFPIELLPLGYSEPAFGPCFSMPRGKPPAPSPRGQKASPPTLAPATASPPTTAGSNNPCLTQLLTAAKPEQALEPPLVSSTLLRSPGSPQETVPEFPCTFLPPTPAPTPPRPPPGPATLAPSRPLLVPKAERLSPPAPSGSERRLSGDLSSMPGPGTLSVRVSPPQPILSRGRPDSNKNRRITHISAEQKRRFNIKLGFDTLHGLVSTLSAQPSLKVSKATTLQKTAEYILMLQQERAGLQEEAQQLRDEIEELNAAINLCQQQLPATGVPITHQRFDQMRDMFDDYVRTRTLHNWKFWVFSILIRPLFESFNGMVSTASVHTLRQTSLAWLDQYCSLPALRPTVLNSLRQLGTSTSILTDPGRIPEQATRAVTEGTLGKPL

SEQ ID NO: 9 - DNA, 3189 bp, Homo sapiens
agggaccaggcggttgcggcggcgacagccatggccggcgcgctggcaggtctggccgcgggcttgcaggtcccgcgggtcgcgcccagcccagactcggactcggacacagactcggaggacccgagtctccggcgcagcgcgggcggcttgctccgctcgcaggtcatccacagcggtcacttcatggtgtcgtcgccgcacagcgactcgctgccccggcggcgcgaccaggaggggtccgtggggccctccgacttcgggccgcgcagtatcgaccccacactcacacgcctcttcgagtgcttgagcctggcctacagtggcaagctggtgtctcccaagtggaagaatttcaaaggcctcaagctgctctgcagagacaagatccgcctgaacaacgccatctggagggcctggtatatccagtatgtgaagcggaggaagagccccgtgtgtggcttcgtgacccccctgcaggggcctgaggctgatgcgcaccggaagccggaggccgtggtcctggaggggaactactggaagcggcgcatcgaggtggtgatgcgggaataccacaagtggcgcatctactacaagaagcggctccgtaagcccagcagggaagatgacctcctggcccctaagcaggcggaaggcaggtggccgccgccggagcaatggtgcaaacagctcttctccagtgtggtccccgtgctgctgggggacccagaggaggagccgggtgggcggcagctcctggacctcaattgctttttgtccgacatctcagacactctcttcaccatgactcagtccggcccttcgcccctgcagctgccgcctgaggatgcctacgtcggcaatgctgacatgatccagccggacctgacgccactgcagccaagcctggatgacttcatggacatctcagatttctttaccaactcccgcctcccacagccgcccatgccttcaaacttcccagagccccccagcttcagccccgtggttgactccctcttcagcagtgggaccctgggcccagaggtgcccccggcttcctcggccatgacccacctctctggacacagccgtctgcaggctcggaacagctgccctggccccttggactccagcgccttcctgagttctgatttcctccttcctgaagaccccaagccccggctcccaccccctcctgtacccccacctctgctgcattaccctccccctgccaaggtgccaggcctggagccctgccccccacctcccttccctcccatggcaccacccactgctttgctgcaggaagagcctctcttctctcccaggtttcccttccccaccgtccctcctgccccaggagtgtctccgctgcctgctcctgcagccttcccacccaccccacagtctgtccccagcccagcccccacccccttccccatagagcttctacccttggggtattcggagcctgcctttgggccttgcttctccatgcccagaggcaagccccccgccccatcccctaggggacagaaagccagcccccctaccttagcccctgccactgccagtccccccaccactgcggggagcaacaacccctgcctcacacagctgctcacagcagctaagccggagcaagccctggagccaccacttgtatccagcaccctcctccggtccccagggtccccgcaggagacagtccctgaattcccctgcacattccttcccccgaccccggcccctacaccgccccggccacctccaggcccggccacattggccccttccaggcccctgcttgtccccaaagcggagcggctctcacccccagcgcccagcggcagtgaacggcggctgtcaggggacctcagctccatgccaggccctgggactctgagcgtccgtgtctctcccccgcaacccatcctcagccggggccgtccagacagcaacaagaaccggcgtatcacacacatctccgcggagcagaagcggcgcttcaacatcaagctggggtttgacacccttcatgggctcgtgagcacactcagtgcccagcccagcctcaaggagcgtgcgggcttgcaggaggaggcccagcagctgcgggatgagattgaggagctcaatgccgccattaacctgtgccagcagcagctgcccgccacaggggtacccatcacacaccagcgttttgaccagatgcgagacatgtttgatgactacgtccgaacccgtacgctgcacaactggaagttctgggtgttcagcatcctcatccggcctctgtttgagtccttcaacgggatggtgtccacggcaagtgtgcacaccctccgccagacctcactggcctggctggaccagtactgctctctgcccgctctccggccaactgtcctgaactccctacgccagctgggcacatctaccagtatcctgaccgacccgggccgcatccctgagcaagccacacgggcagtcacagagggcacccttggcaaacctttatagtcctggccagaccctgctgctcactcagctgccctgggggctgctttccctgggcacgggctccagggatcatctctgggcactcccttcctgccccaggccctggctctgcccttccctggggggtggagcagggtccaggtttcacacttgccacctcctggaggtcaagaagagcagagtccccgtccctgctctgccactgtgctccagcaccgtgaccttgggtgactcgtccgctgtctttggaccgctgtgtttcaatctgcaaaatggggatggggaaggttcaatcagcagatgacccccaggccttggcagctgtgacattgggggcctaggctggcaactccgggggctcaacggtggaaagaggaggatgctgtttctctgtcacctccacttgctccccgacaggtggggcacagacctctgttcctgagcagagaagcagaaaaggaggttccctctctctgctccttcactgctgacccagaggggctgcaggatggtttcccctgggagaggccaggagggcctgatcccaggagacaccagggccagagtgaccacagcagggcaggcatcatgtgtgtgtgtgtgtgtggatgtgtgtgtgtgggttttgtaaagaattcttgaccaataaaagcaaaaactgtc

SEQ ID NO: 10 - protein, 831 aa, Homo sapiens
MAGALAGLAAGLQVPRVAPSPDSDSDTDSEDPSLRRSAGGLLRSQVIHSGHFMVSSPHSDSLPRRRDQEGSVGPSDFGPRSIDPTLTRLFECLSLAYSGKLVSPKWKNFKGLKLLCRDKIRLNNAIWRAWYIQYVKRRKSPVCGFVTPLQGPEADAHRKPEAVVLEGNYWKRRIEVVMREYHKWRIYYKKRLRKPSREDDLLAPKQAEGRWPPPEQWCKQLFSSVVPVLLGDPEEEPGGRQLLDLNCFLSDISDTLFTMTQSGPSPLQLPPEDAYVGNADMIQPDLTPLQPSLDDFMDISDFFTNSRLPQPPMPSNFPEPPSFSPVVDSLFSSGTLGPEVPPASSAMTHLSGHSRLQARNSCPGPLDSSAFLSSDFLLPEDPKPRLPPPPVPPPLLHYPPPAKVPGLEPCPPPPFPPMAPPTALLQEEPLFSPRFPFPTVPPAPGVSPLPAPAAFPPTPQSVPSPAPTPFPIELLPLGYSEPAFGPCFSMPRGKPPAPSPRGQKASPPTLAPATASPPTTAGSNNPCLTQLLTAAKPEQALEPPLVSSTLLRSPGSPQETVPEFPCTFLPPTPAPTPPRPPPGPATLAPSRPLLVPKAERLSPPAPSGSERRLSGDLSSMPGPGTLSVRVSPPQPILSRGRPDSNKNRRITHISAEQKRRFNIKLGFDTLHGLVSTLSAQPSLKERAGLQEEAQQLRDEIEELNAAINLCQQQLPATGVPITHQRFDQMRDMFDDYVRTRTLHNWKFWVFSILIRPLFESFNGMVSTASVHTLRQTSLAWLDQYCSLPALRPTVLNSLRQLGTSTSILTDPGRIPEQATRAVTEGTLGKPL

SEQ ID NO: 11 - DNA, 2335 bp, Homo sapiens
gctcacaactgtctgctgcgcccgaaaaacaagtcggtgcgctggggacccggggccggggccgccttactccggcctagccccgcggccctcggtgcgggctccagggcatgctcgggaccccccgcggctccagcccagacgccccggcctcaggtctcggcccccgcttggggccccggccgtgcggccggagggagcggccggatggagcggaggatgaaagccggatacttggaccagcaagtgccctacaccttcagcagcaaatcgcccggaaatgggagcttgcgcgaagcgctgatcggcccgctggggaagctcatggacccgggctccctgccgcccctcgactctgaagatctcttccaggatctaagtcacttccaggagacgtggctcgctgaagctcaggtaccagacagtgatgagcagtttgttcctgatttccattcagaaaacctagctttccacagccccaccaccaggatcaagaaggagccccagagtccccgcacagacccggccctgtcctgcagcaggaagccgccactcccctaccaccatggcgagcagtgcctttactccagtgcctatgacccccccagacaaatcgccatcaagtcccctgcccctggtgcccttggacagtcgcccctacagccctttccccgggcagagcaacggaatttcctgagatcctctggcacctcccagccccaccctggccatgggtacctcggggaacatagctccgtcttccagcagcccctggacatttgccactccttcacatctcagggagggggccgggaacccctcccagccccctaccaacaccagctgtcggagccctgcccaccctatccccagcagagctttaagcaagaataccatgatcccctgtatgaacaggcgggccagccagccgtggaccagggtggggtcaatgggcacaggtacccaggggcgggggtggtgatcaaacaggaacagacggacttcgcctacgactcagatgtcaccgggtgcgcatcaatgtacctccacacagagggcttctctgggccctctccaggtgacggggccatgggctatggctatgagaaacctctgcgaccattcccagatgatgtctgcgttgtccctgagaaatttgaaggagacatcaagcaggaaggggtcggtgcatttcgagaggggccgccctaccagcgccggggtgccctgcagctgtggcaatttctggtggccttgctggatgacccaacaaatgcccatttcattgcctggacgggccggggaatggagttcaagctcattgagcctgaggaggtcgccaggctctggggcatccagaagaaccggccagccatgaattacgacaagctgagccgctcgctccgatactattatgagaaaggcatcatgcagaaggtggctggtgagcgttacgtgtacaagtttgtgtgtgagcccgaggccctcttctctttggccttcccggacaatcagcgtccagctctcaaggctgagtttgaccggcctgtcagtgaggaggacacagtccctttgtcccacttggatgagagccccgcctacctcccagagctggctggccccgcccagccatttggccccaagggtggctactcttactagcccccagcggctgttccccctgccgcaggtgggtgctgccctgtgtacatataaatgaatctggtgttggggaaaccttcatctgaaacccacagatgtctctggggcagatccccactgtcctaccagttgccctagcccagactctgagctgctcaccggagtcattgggaaggaaaagtggagaaatggcaagtctagagtctcagaaactcccctgggggtttcacctgggccctggaggaattcagctcagcttcttcctaggtccaagccccccacaccttttccccaaccacagagaacaagagtttgttctgttctgggggacagagaaggcgcttcccaacttcatactggcaggagggtgaggaggttcactgagctccccagatctcccactgcggggagacagaagcctggactctgccccacgctgtggccctggagggtcccggtttgtcagttcttggtgctctgtgttcccagaggcaggcggaggttgaagaaaggaacctgggatgaggggtgctgggtataagcagagagggatgggttcctgctccaagggaccctttgcctttcttctgccctttcctaggcccaggcctgggtttgtacttccacctccaccacatctgccagaccttaataaaggcccccacttctccca

SEQ ID NO: 12 - protein, 484 aa, Homo sapiens
MERRMKAGYLDQQVPYTFSSKSPGNGSLREALIGPLGKLMDPGSLPPLDSEDLFQDLSHFQETWLAEAQVPDSDEQFVPDFHSENLAFHSPTTRIKKEPQSPRTDPALSCSRKPPLPYHHGEQCLYSSAYDPPRQIAIKSPAPGALGQSPLQPFPRAEQRNFLRSSGTSQPHPGHGYLGEHSSVFQQPLDICHSFTSQGGGREPLPAPYQHQLSEPCPPYPQQSFKQEYHDPLYEQAGQPAVDQGGVNGHRYPGAGVVIKQEQTDFAYDSDVTGCASMYLHTEGFSGPSPGDGAMGYGYEKPLRPFPDDVCVVPEKFEGDIKQEGVGAFREGPPYQRRGALQLWQFLVALLDDPTNAHFIAWTGRGMEFKLIEPEEVARLWGIQKNRPAMNYDKLSRSLRYYYEKGIMQKVAGERYVYKFVCEPEALFSLAFPDNQRPALKAEFDRPVSEEDTVPLSHLDESPAYLPELAGPAQPFGPKGGYSY

SEQ ID NO: 13 - DNA, 2224 bp, Homo sapiens
gctcacaactgtctgctgcgcccgaaaaacaagtcggtgcgctggggacccggggccggggccgccttactccggcctagccccgcggccctcggtgcgggctccagggcatgctcgggaccccccgcggctccagcccagacgccccggcctcagaaatcgcccggaaatgggagcttgcgcgaagcgctgatcggcccgctggggaagctcatggacccgggctccctgccgcccctcgactctgaagatctcttccaggatctaagtcacttccaggagacgtggctcgctgaagctcaggtaccagacagtgatgagcagtttgttcctgatttccattcagaaaacctagctttccacagccccaccaccaggatcaagaaggagccccagagtccccgcacagacccggccctgtcctgcagcaggaagccgccactcccctaccaccatggcgagcagtgcctttactccagtgcctatgacccccccagacaaatcgccatcaagtcccctgcccctggtgcccttggacagtcgcccctacagccctttccccgggcagagcaacggaatttcctgagatcctctggcacctcccagccccaccctggccatgggtacctcggggaacatagctccgtcttccagcagcccctggacatttgccactccttcacatctcagggagggggccgggaacccctcccagccccctaccaacaccagctgtcggagccctgcccaccctatccccagcagagctttaagcaagaataccatgatcccctgtatgaacaggcgggccagccagccgtggaccagggtggggtcaatgggcacaggtacccaggggcgggggtggtgatcaaacaggaacagacggacttcgcctacgactcagatgtcaccgggtgcgcatcaatgtacctccacacagagggcttctctgggccctctccaggtgacggggccatgggctatggctatgagaaacctctgcgaccattcccagatgatgtctgcgttgtccctgagaaatttgaaggagacatcaagcaggaaggggtcggtgcatttcgagaggggccgccctaccagcgccggggtgccctgcagctgtggcaatttctggtggccttgctggatgacccaacaaatgcccatttcattgcctggacgggccggggaatggagttcaagctcattgagcctgaggaggtcgccaggctctggggcatccagaagaaccggccagccatgaattacgacaagctgagccgctcgctccgatactattatgagaaaggcatcatgcagaaggtggctggtgagcgttacgtgtacaagtttgtgtgtgagcccgaggccctcttctctttggccttcccggacaatcagcgtccagctctcaaggctgagtttgaccggcctgtcagtgaggaggacacagtccctttgtcccacttggatgagagccccgcctacctcccagagctggctggccccgcccagccatttggccccaagggtggctactcttactagcccccagcggctgttccccctgccgcaggtgggtgctgccctgtgtacatataaatgaatctggtgttggggaaaccttcatctgaaacccacagatgtctctggggcagatccccactgtcctaccagttgccctagcccagactctgagctgctcaccggagtcattgggaaggaaaagtggagaaatggcaagtctagagtctcagaaactcccctgggggtttcacctgggccctggaggaattcagctcagcttcttcctaggtccaagccccccacaccttttccccaaccacagagaacaagagtttgttctgttctgggggacagagaaggcgcttcccaacttcatactggcaggagggtgaggaggttcactgagctccccagatctcccactgcggggagacagaagcctggactctgccccacgctgtggccctggagggtcccggtttgtcagttcttggtgctctgtgttcccagaggcaggcggaggttgaagaaaggaacctgggatgaggggtgctgggtataagcagagagggatgggttcctgctccaagggaccctttgcctttcttctgccctttcctaggcccaggcctgggtttgtacttccacctccaccacatctgccagaccttaataaaggcccccacttctccca

SEQ ID NO: 14 - protein, 445 aa, Homo sapiens
MDPGSLPPLDSEDLFQDLSHFQETWLAEAQVPDSDEQFVPDFHSENLAFHSPTTRIKKEPQSPRTDPALSCSRKPPLPYHHGEQCLYSSAYDPPRQIAIKSPAPGALGQSPLQPFPRAEQRNFLRSSGTSQPHPGHGYLGEHSSVFQQPLDICHSFTSQGGGREPLPAPYQHQLSEPCPPYPQQSFKQEYHDPLYEQAGQPAVDQGGVNGHRYPGAGVVIKQEQTDFAYDSDVTGCASMYLHTEGFSGPSPGDGAMGYGYEKPLRPFPDDVCVVPEKFEGDIKQEGVGAFREGPPYQRRGALQLWQFLVALLDDPTNAHFIAWTGRGMEFKLIEPEEVARLWGIQKNRPAMNYDKLSRSLRYYYEKGIMQKVAGERYVYKFVCEPEALFSLAFPDNQRPALKAEFDRPVSEEDTVPLSHLDESPAYLPELAGPAQPFGPKGGYSY

SEQ ID NO: 15 - DNA, 1571 bp, Homo sapiens
gcttgcccagcccccgctgctgccttccatggcctcagccgcagccctcaagttgaggaggggttccagcatcacactccctctgggtgaactttccctgggattttgtggttggcaggcaacctgggcaaagaacagtcaccaggaagcaggctggaaggaagaaattcttgaatgtggataggacttcctcctcccctgccctcgagctccaccccaagccacttctcacatcaccccttcttcccccacagatgtcaccgggtgcgcatcaatgtacctccacacagagggcttctctgggccctctccaggtgacggggccatgggctatggctatgagaaacctctgcgaccattcccagatgatgtctgcgttgtccctgagaaatttgaaggagacatcaagcaggaaggggtcggtgcatttcgagaggggccgccctaccagcgccggggtgccctgcagctgtggcaatttctggtggccttgctggatgacccaacaaatgcccatttcattgcctggacgggccggggaatggagttcaagctcattgagcctgaggaggtcgccaggctctggggcatccagaagaaccggccagccatgaattacgacaagctgagccgctcgctccgatactattatgagaaaggcatcatgcagaaggtggctggtgagcgttacgtgtacaagtttgtgtgtgagcccgaggccctcttctctttggccttcccggacaatcagcgtccagctctcaaggctgagtttgaccggcctgtcagtgaggaggacacagtccctttgtcccacttggatgagagccccgcctacctcccagagctggctggccccgcccagccatttggccccaagggtggctactcttactagcccccagcggctgttccccctgccgcaggtgggtgctgccctgtgtacatataaatgaatctggtgttggggaaaccttcatctgaaacccacagatgtctctggggcagatccccactgtcctaccagttgccctagcccagactctgagctgctcaccggagtcattgggaaggaaaagtggagaaatggcaagtctagagtctcagaaactcccctgggggtttcacctgggccctggaggaattcagctcagcttcttcctaggtccaagccccccacaccttttccccaaccacagagaacaagagtttgttctgttctgggggacagagaaggcgcttcccaacttcatactggcaggagggtgaggaggttcactgagctccccagatctcccactgcggggagacagaagcctggactctgccccacgctgtggccctggagggtcccggtttgtcagttcttggtgctctgtgttcccagaggcaggcggaggttgaagaaaggaacctgggatgaggggtgctgggtataagcagagagggatgggttcctgctccaagggaccctttgcctttcttctgccctttcctaggcccaggcctgggtttgtacttccacctccaccacatctgccagaccttaataaaggcccccacttctccca

SEQ ID NO: 16 - protein, 207 aa, Homo sapiens
MYLHTEGFSGPSPGDGAMGYGYEKPLRPFPDDVCVVPEKFEGDIKQEGVGAFREGPPYQRRGALQLWQFLVALLDDPTNAHFIAWTGRGMEFKLIEPEEVARLWGIQKNRPAMNYDKLSRSLRYYYEKGIMQKVAGERYVYKFVCEPEALFSLAFPDNQRPALKAEFDRPVSEEDTVPLSHLDESPAYLPELAGPAQPFGPKGGYSY

SEQ ID NO: 17 - DNA, 2215 bp, Homo sapiens
gcagaaagcagaaacggcgagcccggctcctgggagcaggtctcggcccccgcttggggccccggccgtgcggccggagggagcggccggatggagcggaggatgaaagccggatacttggaccagcaagtgccctacaccttcagcagcaaatcgcccggaaatgggagcttgcgcgaagcgctgatcggcccgctggggaagctcatggacccgggctccctgccgcccctcgactctgaagatctcttccaggatctaagtcacttccaggagacgtggctcgctgaagctcaggtaccagacagtgatgagcagtttgttcctgatttccattcagaaaaccctttccacagccccaccaccaggatcaagaaggagccccagagtccccgcacagacccggccctgtcctgcagcaggaagccgccactcccctaccaccatggcgagcagtgcctttactccagtgcctatgacccccccagacaaatcgccatcaagtcccctgcccctggtgcccttggacagtcgcccctacagccctttccccgggcagagcaacggaatttcctgagatcctctggcacctcccagccccaccctggccatgggtacctcggggaacatagctccgtcttccagcagcccctggacatttgccactccttcacatctcagggagggggccgggaacccctcccagccccctaccaacaccagctgtcggagccctgcccaccctatccccagcagagctttaagcaagaataccatgatcccctgtatgaacaggcgggccagccagccgtggaccagggtggggtcaatgggcacaggtacccaggggcgggggtggtgatcaaacaggaacagacggacttcgcctacgactcagatgtcaccgggtgcgcatcaatgtacctccacacagagggcttctctgggccctctccaggtgacggggccatgggctatggctatgagaaacctctgcgaccattcccagatgatgtctgcgttgtccctgagaaatttgaaggagacatcaagcaggaaggggtcggtgcatttcgagaggggccgccctaccagcgccggggtgccctgcagctgtggcaatttctggtggccttgctggatgacccaacaaatgcccatttcattgcctggacgggccggggaatggagttcaagctcattgagcctgaggaggtcgccaggctctggggcatccagaagaaccggccagccatgaattacgacaagctgagccgctcgctccgatactattatgagaaaggcatcatgcagaaggtggctggtgagcgttacgtgtacaagtttgtgtgtgagcccgaggccctcttctctttggccttcccggacaatcagcgtccagctctcaaggctgagtttgaccggcctgtcagtgaggaggacacagtccctttgtcccacttggatgagagccccgcctacctcccagagctggctggccccgcccagccatttggccccaagggtggctactcttactagcccccagcggctgttccccctgccgcaggtgggtgctgccctgtgtacatataaatgaatctggtgttggggaaaccttcatctgaaacccacagatgtctctggggcagatccccactgtcctaccagttgccctagcccagactctgagctgctcaccggagtcattgggaaggaaaagtggagaaatggcaagtctagagtctcagaaactcccctgggggtttcacctgggccctggaggaattcagctcagcttcttcctaggtccaagccccccacaccttttccccaaccacagagaacaagagtttgttctgttctgggggacagagaaggcgcttcccaacttcatactggcaggagggtgaggaggttcactgagctccccagatctcccactgcggggagacagaagcctggactctgccccacgctgtggccctggagggtcccggtttgtcagttcttggtgctctgtgttcccagaggcaggcggaggttgaagaaaggaacctgggatgaggggtgctgggtataagcagagagggatgggttcctgctccaagggaccctttgcctttcttctgccctttcctaggcccaggcctgggtttgtacttccacctccaccacatctgccagaccttaataaaggcccccacttctccca

SEQ ID NO: 18 - protein, 483 aa, Homo sapiens
MERRMKAGYLDQQVPYTFSSKSPGNGSLREALIGPLGKLMDPGSLPPLDSEDLFQDLSHFQETWLAEAQVPDSDEQFVPDFHSENPFHSPTTRIKKEPQSPRTDPALSCSRKPPLPYHHGEQCLYSSAYDPPRQIAIKSPAPGALGQSPLQPFPRAEQRNFLRSSGTSQPHPGHGYLGEHSSVFQQPLDICHSFTSQGGGREPLPAPYQHQLSEPCPPYPQQSFKQEYHDPLYEQAGQPAVDQGGVNGHRYPGAGVVIKQEQTDFAYDSDVTGCASMYLHTEGFSGPSPGDGAMGYGYEKPLRPFPDDVCVVPEKFEGDIKQEGVGAFREGPPYQRRGALQLWQFLVALLDDPTNAHFIAWTGRGMEFKLIEPEEVARLWGIQKNRPAMNYDKLSRSLRYYYEKGIMQKVAGERYVYKFVCEPEALFSLAFPDNQRPALKAEFDRPVSEEDTVPLSHLDESPAYLPELAGPAQPFGPKGGYSY

SEQ ID NO: 19 - DNA, 2203 bp, Homo sapiens
gcagaaagcagaaacggcgagcccggctcctgggagcaggtctcggcccccgcttggggccccggccgtgcggccggagggagcggccggatggagcggaggatgaaagccggatacttggaccagcaagtgccctacaccttcagcagcaaatcgcccggaaatgggagcttgcgcgaagcgctgatcggcccgctggggaagctcatggacccgggctccctgccgcccctcgactctgaagatctcttccaggatctaagtcacttccaggagacgtggctcgctgaagctcaggtaccagacagtgatgagcagtttgttcctgatttccattcagaaaacctagctttccacagccccaccaccaggatcaagaaggagccccagagtccccgcacagacccggccctgtcctgcagcaggaagccgccactcccctaccaccatggcgagcagtgcctttactccagtgcctatgacccccccagacaaatcgccatcaagtcccctgcccctggtgcccttggacagtcgcccctacagccctttccccgggcagagcaacggaatttcctgagatcctctggcacctcccagccccaccctggccatgggtacctcggggaacatagctccgtcttccagcagcccctggacatttgccactccttcacatctcagggagggggccgggaacccctcccagccccctaccaacaccagctgtcggagccctgcccaccctatccccagcagagctttaagcaagaataccatgatcccctgtatgaacaggcgggccagccagccgtggaccagggtggggtcaatgggcacaggtacccaggggcgggggtggtgatcaaacaggaacagacggacttcgcctacgactcagatgtcaccgggtgcgcatcaatgtacctccacacagagggcttctctgggccctctccaggctatggctatgagaaacctctgcgaccattcccagatgatgtctgcgttgtccctgagaaatttgaaggagacatcaagcaggaaggggtcggtgcatttcgagaggggccgccctaccagcgccggggtgccctgcagctgtggcaatttctggtggccttgctggatgacccaacaaatgcccatttcattgcctggacgggccggggaatggagttcaagctcattgagcctgaggaggtcgccaggctctggggcatccagaagaaccggccagccatgaattacgacaagctgagccgctcgctccgatactattatgagaaaggcatcatgcagaaggtggctggtgagcgttacgtgtacaagtttgtgtgtgagcccgaggccctcttctctttggccttcccggacaatcagcgtccagctctcaaggctgagtttgaccggcctgtcagtgaggaggacacagtccctttgtcccacttggatgagagccccgcctacctcccagagctggctggccccgcccagccatttggccccaagggtggctactcttactagcccccagcggctgttccccctgccgcaggtgggtgctgccctgtgtacatataaatgaatctggtgttggggaaaccttcatctgaaacccacagatgtctctggggcagatccccactgtcctaccagttgccctagcccagactctgagctgctcaccggagtcattgggaaggaaaagtggagaaatggcaagtctagagtctcagaaactcccctgggggtttcacctgggccctggaggaattcagctcagcttcttcctaggtccaagccccccacaccttttccccaaccacagagaacaagagtttgttctgttctgggggacagagaaggcgcttcccaacttcatactggcaggagggtgaggaggttcactgagctccccagatctcccactgcggggagacagaagcctggactctgccccacgctgtggccctggagggtcccggtttgtcagttcttggtgctctgtgttcccagaggcaggcggaggttgaagaaaggaacctgggatgaggggtgctgggtataagcagagagggatgggttcctgctccaagggaccctttgcctttcttctgccctttcctaggcccaggcctgggtttgtacttccacctccaccacatctgccagaccttaataaaggcccccacttctccca

SEQ ID NO: 20 - protein, 479 aa, Homo sapiens
MERRMKAGYLDQQVPYTFSSKSPGNGSLREALIGPLGKLMDPGSLPPLDSEDLFQDLSHFQETWLAEAQVPDSDEQFVPDFHSENLAFHSPTTRIKKEPQSPRTDPALSCSRKPPLPYHHGEQCLYSSAYDPPRQIAIKSPAPGALGQSPLQPFPRAEQRNFLRSSGTSQPHPGHGYLGEHSSVFQQPLDICHSFTSQGGGREPLPAPYQHQLSEPCPPYPQQSFKQEYHDPLYEQAGQPAVDQGGVNGHRYPGAGVVIKQEQTDFAYDSDVTGCASMYLHTEGFSGPSPGYGYEKPLRPFPDDVCVVPEKFEGDIKQEGVGAFREGPPYQRRGALQLWQFLVALLDDPTNAHFIAWTGRGMEFKLIEPEEVARLWGIQKNRPAMNYDKLSRSLRYYYEKGIMQKVAGERYVYKFVCEPEALFSLAFPDNQRPALKAEFDRPVSEEDTVPLSHLDESPAYLPELAGPAQPFGPKGGYSY

SEQ ID NO: 21 - DNA, 2371 bp, Homo sapiens
gaaagcagttctctgggaccaccttcttttggcttcaacctctcccactcttgacatctgagtagctcagggaagctcttccaggtccgactgttcatatgtaaaggagactggccgctggggctcaggaccgggattatccgagctctgcagaagtgcaccgctattgctttgggaggttaaaaaaaaaatcacacggtttccagtgaaaaagtgacagagggtggtggcctttggaaccgccgtgaagtcttctgcctggaacccgaaacttgcatgctatggaacacccgctctttggctgcctgcgcagccctcacgccacggcgcaaggcttgcacccgttctcccaatcctctctcgccctccatggaagatctgaccatatgtcttaccccgagctctctacttcttcctcatcttgcataatcgcgggataccccaacgaagagggcatgtttgccagccagcatcacagggggcaccaccaccaccaccaccaccaccaccatcaccaccatcagcagcagcagcaccaggctctgcaaaccaactggcacctcccgcagatgtcttccccaccgagtgcggctcggcacagcctctgcctccagcccgactctggagggcccccagagttggggagcagcccgcccgtcctgtgctccaactcttccagcttgggctccagcaccccgactggggccgcgtgcgcgccgggggactacggccgccaggcactgtcacctgcggaggcggagaagcgaagcggcggcaagaggaaaagcgacagctcagactcccaggaaggaaattacaagtcagaagtcaacagcaaacccaggaaagaaaggacagcatttaccaaagagcaaatcagagaacttgaagcagaatttgcccatcataattatctcaccagactgaggcgatacgagatagcagtgaatctggatctcactgaaagacaggtgaaagtctggttccaaaacaggcggatgaagtggaagagggtaaagggtggacagcaaggagctgcggctcgggaaaaggaactggtgaatgtgaaaaagggaacacttctcccatcagagctgtcgggaattggtgcagccaccctccagcaaacaggggactctatagcaaatgaagacagtcacgacagtgaccacagctcagagcatgcgcacttatgatataaacagaggaccagctccattctcaggaaagaaatgttgtgatggcaagccttacccaaatatcgtttacacagagagatgactatggcagtgatgtttaatattattaaatccaggcatttcgaatctgtttttcatgatttatagagggtttacacaaagtgccacttattaaagagcttccacagtgaagatggagaaggtgaacttgctttgaatattccagatgtgtttggtcgtgcgtatggcagtgagcaggtatgtgtttgcttttgcttgcactgaaaattaaattgctatcaagagcaaactatgaacggttttttattcaagatgtctccagagtgaagatgccgaggatgaacttgcattgaacattccagatgtgtgagatcatgtgtattacagtgggcaggtatttgcttttgcttgcactgaaaattaaattgctatcaagaataaaccatgaaacattttatcctgaacagccacagtgcctgaattcactcaagtggataaaaagtgtattttaactctgtatatattacccttaagtcattttcctgtcttcactaatttagcaatgcattcatattagctgatgaaaataggcactcacaatgacaaccagagccagtttcttgtcttttttatacattttgtcatcccagagacaatcagtatgtgcttacctgtgttcaagtagagaaaaatacagtagagtctgataggacatattcttgtaccacagacaaaacaaatcttatgttgcatttactatcaactgctgctaatacgttattataaaacttacctagctcctgaattcttcctatcttatagcttaaaacaattaggatcataggcaaatcagttaccttgcagaaagagctttgtatgacagacattgtcttattttatttctgtaaaatattagctgtatgaatatgatttaattaacaagaaaacatttcttcctgattgacaacagtgttagacaaggtgcaaagcgaaactggttgctcaagttgatagaaaacaaaattctgaatatcttcaaattaaattcggtaaaaacacattattttttcatatgtgatgtattcatgcagaacaactatctttgtattttgtttttaaaatgtgtttaataaatgatcctttgtaaataa

SEQ ID NO: 22 - protein, 304 aa, Homo sapiens
MEHPLFGCLRSPHATAQGLHPFSQSSLALHGRSDHMSYPELSTSSSSCIIAGYPNEEGMFASQHHRGHHHHHHHHHHHHHQQQQHQALQTNWHLPQMSSPPSAARHSLCLQPDSGGPPELGSSPPVLCSNSSSLGSSTPTGAACAPGDYGRQALSPAEAEKRSGGKRKSDSSDSQEGNYKSEVNSKPRKERTAFTKEQIRELEAEFAHHNYLTRLRRYEIAVNLDLTERQVKVWFQNRRMKWKRVKGGQQGAAAREKELVNVKKGTLLPSELSGIGAATLQQTGDSIANEDSHDSDHSSEHAHL

SEQ ID NO: 23 - DNA, 2707 bp, Homo sapiens
ggacgagcggcagcagctgggcgagtgacagccccggctccgcgcgccgcggccgccagagccggcgcaggggaagcgcccgcggccccgggtgcagcagcggccgccgcctcccgcgcctccccggcccgcagcccgcggtcccgcggccccggggccggcacctctcgggctccggctccccgcgcgcaagatggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagctagagacaagagagacacctccaacttcgacaaagagttcaccagacagcctgtggaactgacccccactgataaactcttcatcatgaacttggaccaaaatgaatttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagagggcttttctttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtttttacagttattctataatatcttctttgaatgctaagcatgagcgatatttttaaaaattgtgagtaagctttgcagttactgtgaactattgtctcttggaggaagttttttgtttaagaattgatatgattaaactgaattaatatatgcaa

SEQ ID NO: 24 - protein, 671 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKARDKRDTSNFDKEFTRQPVELTPTDKLFIMNLDQNEFAGFSYTNPEFVINV

SEQ ID NO: 25 - DNA, 8010 bp, Homo sapiens
ggacgagcggcagcagctgggcgagtgacagccccggctccgcgcgccgcggccgccagagccggcgcaggggaagcgcccgcggccccgggtgcagcagcggccgccgcctcccgcgcctccccggcccgcagcccgcggtcccgcggccccggggccggcacctctcgggctccggctccccgcgcgcaagatggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggggcttcgggaagcagggattccagtgccaagtttgctgctttgtggtgcacaagcggtgccatgaatttgtcacattctcctgccctggcgctgacaagggtccagcctccgatgacccccgcagcaaacacaagtttaagatccacacgtactccagccccacgttttgtgaccactgtgggtcactgctgtatggactcatccaccaggggatgaaatgtgacacctgcatgatgaatgtgcacaagcgctgcgtgatgaatgttcccagcctgtgtggcacggaccacacggagcgccgcggccgcatctacatccaggcccacatcgacagggacgtcctcattgtcctcgtaagagatgctaaaaaccttgtacctatggaccccaatggcctgtcagatccctacgtaaaactgaaactgattcccgatcccaaaagtgagagcaaacagaagaccaaaaccatcaaatgctccctcaaccctgagtggaatgagacatttagatttcagctgaaagaatcggacaaagacagaagactgtcagtagagatttgggattgggatttgaccagcaggaatgacttcatgggatctttgtcctttgggatttctgaacttcagaaagccagtgttgatggctggtttaagttactgagccaggaggaaggcgagtacttcaatgtgcctgtgccaccagaaggaagtgaggccaatgaagaactgcggcagaaatttgagagggccaagatcagtcagggaaccaaggtcccggaagaaaagacgaccaacactgtctccaaatttgacaacaatggcaacagagaccggatgaaactgaccgattttaacttcctaatggtgctggggaaaggcagctttggcaaggtcatgctttcagaacgaaaaggcacagatgagctctatgctgtgaagatcctgaagaaggacgttgtgatccaagatgatgacgtggagtgcactatggtggagaagcgggtgttggccctgcctgggaagccgcccttcctgacccagctccactcctgcttccagaccatggaccgcctgtactttgtgatggagtacgtgaatgggggcgacctcatgtatcacatccagcaagtcggccggttcaaggagccccatgctgtattttacgctgcagaaattgccatcggtctgttcttcttacagagtaagggcatcatttaccgtgacctaaaacttgacaacgtgatgctcgattctgagggacacatcaagattgccgattttggcatgtgtaaggaaaacatctgggatggggtgacaaccaagacattctgtggcactccagactacatcgcccccgagataattgcttatcagccctatgggaagtccgtggattggtgggcatttggagtcctgctgtatgaaatgttggctgggcaggcaccctttgaaggggaggatgaagatgaactcttccaatccatcatggaacacaacgtagcctatcccaagtctatgtccaaggaagctgtggccatctgcaaagggctgatgaccaaacacccaggcaaacgtctgggttgtggacctgaaggcgaacgtgatatcaaagagcatgcatttttccggtatattgattgggagaaacttgaacgcaaagagatccagcccccttataagccaaaagcttgtgggcgaaatgctgaaaacttcgaccgatttttcacccgccatccaccagtcctaacacctcccgaccaggaagtcatcaggaatattgaccaatcagaattcgaaggattttcctttgttaactctgaatttttaaaacccgaagtcaagagctaagtagatgtgtagatctccgtccttcatttctgtcattcaagctcaacggctattgtggtgacatttttatgtttttcattgccaagttgcatccatgtttgattttctgatgagactagagtgacagtgtttcagaacccaaatgtcctcaggtagtttggagcatctctatgagatgggattatgcagatggcctatggaaaatgcagctgcataattaacacattatcaaagtcctcttacaatttattttccgcagcatgtcagctaagtagacccaatggggagagaaaatgcctgctttctttccctctttttctgcactgccatattcacccccaaccatccaatctgtggataattggatgttagcggtactcttccacttccgggcctggagcttggcttgtatccaagtgtatggttgctttgcctaagaggaatccctctatttcacctgttctggaggcaccagaccttgaaaagaacatgctcaaaataaaatgttatctgttatttttgtaaactcaaagttaagatgatcaaagttctaaaattccaagaatgtgcttttagacggtctcaatctaaaagcacttcaaggggtcaaagggcaaccagcttgggtgctacctcagtgttgtagtttctgatactttatgtctttgctcaccctcatccccaaactacttgaaaagggcatttggcaccactctctgaaacaacacagtcactctagcaaggcccccaaagggccctggttttacattacatttcaaactttatttgctttggggttttgtttctgttgttgttcaaatgcaaaaaaaagaaaaaaaaagaaaaaaaaaggtgactcacattgttacacatgctttaaaatatgtattcaaatgttattaaccacaatgacgacctgctttgatttaaccaagaagacggctgcggagcctagcagactcaggcctgtgggaatgggatttgttacaaatctaggtttgttactggcttcagaaagctaattaagtgctctgaaaaagacaccgtttcttgaaacaaagatggttgtattcctcactttgatgttgttttgcaagatgtttgtggaaatgttcatttgtatctggatctctgttatgtgccatttttcttctagcatcgagatacaataaaaaaaaaaaaaaagaaaagaagaagaaatactatttcaaggaaaactgctctttttgagaaacgtggacctaaactacaaagtgggaactgaggagggaactcaggagaaaggaactaactgcggagctttaatcttggccccagtgttcagccactcggaggggcgggggctgtggcccattcaggggctgctggtgggctgtagtggggtgggatgacctggccagagccaacgaggatactggagcccaaagtcaagtttagagaccagctgggaacgtgaatggggctcttgattttcttatcaaaatcaccactcctcccagcttggactaaatattctttctagcaagcagctttgtgagctccctgaagcccaaggaaacccttcggtgggagaaatttcatttctgtctgagaggattaaggcagcaggtgactccccctcctcgcctgccgtgtcctgctattctcaggcagctctaaggagaattcttatcacagttcaagtgatttccagaagttccagggcttctgagagaccatcaagggaactttaacaacttgacaaatgtccttgaagtaagatgcctcatctttagggaaaaatggggtttggatttctgcttaggcaaagtctcctgcagttcatccttctctgtcctcttcttgcttcaggcttggggaccgtccctgctgtccccactgtggtggcaatcaggacctaaggtgaagcaaacttgaagttctatctgacaagtttaggcagtaagagaaggagggaaatcggagcaaagctccctcactttattgttgagaaactggcatctggaaagaggaaggaatttgcccaaagtcagtcagctgggataaaaacctgggtgtcctgtccagaaagtgcagggtgctttctgctctgtagcaaggcagcagacatctctgagccaggcccaccaacaggcccttatctggtggttggatcatgatcccattttgcttggacatgctctcaggaagataaaaaccatggagaaacactaggccattgacaaatgatctgagacaactttagaaaacaatgtaggatgaatggaaagagaaagaaaggaaagaaagaagaaaaagaaagaaggaaagaaagaaagagaaaggaaggaaggaaagaaggaaggaaaagaaggaaggaaggaaggaatatagtgttataaatactgcactcaacattttccaaattcttgccattatttttcaaaagtttaatagtttgcagaaatagatactcaagccaaagtctgttttagagaaactttccatggaaagtcagaatttctaccacttccttttctatccacatttccagtgcagaagaaactgagaaacagagctttttgaagagaggacagggccatagcaacaaggaccttcttgggggattaatgggaggtcagtagaattaataaccctccttggatgagtgctactgttttcacatggcttcagatgctatcaacctcaaagaaatgatctcaacagagaagcttattctctcccaacttctacggtaaaatccaggagtattttctctggggatctgcccacaggacaaagtccataaaagcaagtcctgtctggaccatgtggttatctgaagcattagccatcaccagcacaacaaacggggcagggctttccaaggtggggctggtcagaagggaatctttgataagaggcccacaggcagggaaagcgaaatagggttgatgagaccaggggagacctaaaaaaaaggcagctttgtgtcttctagctccaaatatacctgccttttagctcacacactgtcctggagttctcagacctttaggggccctaacacagttcagttcatacaggggttcaaaagggacagtggcccatttgggagacctttaggatcaatgggaatcaattccattgttttgcctcagagtaaagtttctggctcggggacaattataagttgcaaaaaggatagaggcatatcccaagtcttccttcattccacaaataattacaaacaacctactgtgtgccaggcactattcttagcactggaaatacactagtgaagaagcagatgaggaccctgtttattgtttctctccaagaaattctccaagaatattgtttcttggagagaaataataaataaacaagacaatttctgaaagcaataagtgcaatcaagataattaaaggatgctaaagtgtgacttgtggggattgggagagagatgcacagacaatattaaagaggaggcattcgagctttgttgtgaacaccggaagtaacatgccgagcgcctgggggatggaaactcctatagcaccccacaggctaacagcaagcaggacaagacaaaaagggcaggtgggacatggtagagatggaccctacccaggaaacagctccatcagcatcttagcctgccccactctagccacacatacccacgtgtgctcctgagttcagtgtgcccacctcactcccacaccctcacatagacttggcaagagtaaggagggaactccatagagacattttacctatctcaggggagcagccacaaagaagcaagtcttgtaaaaggtcttttgcaaaggagagtgaacccagcaatgagagatccttaacagctagtgcccattagggggctaaacctaaagcctgggtggtgatggctcaaacgctaatgagtcagtgaatccttaccgaccccctggcctttataatctgaggcaactttggctgcagcccgggaatgtgcagggcactagggaatacaaggccttcttccctggttgtcttgtaataaaacagccatggggttgtccctccagtccgagagactgtgatgaggcctacatagcagcgatgtggtcaggtaaaaatcaggaacccactgaaatcttgggcaagccaccctgcctgcttgtgcctcggttctctcatatgtcatatataggaggtgaggactccagctccacctgccccaggtgggtgtggtgatgatgaggaaagacaagaggcttgcaaggaccctgaagaggtcggagcatcatacagattcctttattagcccacattctgatgttccctggtgagacttgccccaagcaattgctagtaaatgggggttaatttcttctccacctccctactgaacaaaaaaagaaatgccagacttactaggagaatcgagttgctttgagtttcttttgttttgttttgttttgttttgttttaaggctccccttacacaccctcctttaagctttgggttttctctcttatagtttgttgacacatgctaaaaatgtctttggagagaacttctgcctgataaacacccaattctagactgtgggtggattttcgagctgacggtggtcaattcctttcattaagcagtgatctgatttctccacatggccattctgccttcttgggggcagagtagatgggcagcagttcaccttttcagagaaagaggtcttctagccacctgggctgctactgaatggttttctccaggacgctctacctaatgattatttctataacattaagcatggtaataagtagcttccaattcaattcatcctaaagccaaagaaaatacagcaacacacacacacacacacacacacacacacacacacacacacacacaccactttatggcaattcttaactgacattcaatgacttacttcttttcttagaaaatttccaccacatttctatccccaagccaacatacaatgtgaaatgaaagccagtgcgtggagtgcagctgctaaaaattttcagcacagggctctttctgactctgctcatgagatggtatcagccacccaatgactggcgtatcttggtcctgtgtctttcttcttacgctgtgttaatgtgtttactttccatttggcagagagacaagagagacacctccaacttcgacaaagagttcaccagacagcctgtggaactgacccccactgataaactcttcatcatgaacttggaccaaaatgaatttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagagggcttttctttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtttttacagttattctataatatcttctttgaatgctaagcatgagcgatatttttaaaaattgtgagtaagctttgcagttactgtgaactattgtctcttggaggaagttttttgtttaagaattgatatgattaaactgaattaatatatgcaa

SEQ ID NO: 26 - protein, 673 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKACGRNAENFDRFFTRHPPVLTPPDQEVIRNIDQSEFEGFSFVNSEFLKPEVKS

SEQ ID NO: 27 - DNA, 3815 bp, Homo sapiens
ggctctgcagtgggcgccggctccctgggctgggagggggctcctggggcgggtgggagggtggggggccggggtggggtggggcaggatgctggatggcccactgttctccgaggggcctgacagcccccgggagctccaggatgaggagtctggcagctgcctctgggtgcagaagtccaagctattggtgatagaagtgaagactatttcctgtcattatagtcgccgcgccccttctcgacagcccatggacttccaggccagccactgggctcgcgggttccagaaccgcacgtgtgggccgcgcccgggatccccacagccgccgccccgccggccctgggcctccagggtgctgcaggaggcgaccaactggcgggcggggcccctggccgaggtccgagctcgggagcaagagaaaaggaaagcggcgtcgcaggagcgggaggccaaggagaccgagcgaaaaaggcgcaaggctggtggggcccgacggagccccccgggtcgaccccgcccggagccccgcaacgcccctcgggtggcccagctggcagggctccctgctcccttgcggccggagcgcctggcgcctgtggggcgagcgccccgtccatccgcgcagccgcagagcgacccagggtcggcgtgggcggggccctggggaggtcggcggcccgggcccccaagctacgaggctcacctgctgctgagaggttctgccgggaccgccccacgacgccgctgggaccggccgccaccctacgtggctccaccttcttacgaaggcccccataggaccttggggactaagagaggccccgggaactctcaggtgcccacttcatcagccccagctgcgactccagccaggacagacggagggcgcacaaagaagaggctggatcctcggatctaccgggacgtcctcggggcttggggtctccgacaggggcaaggtctcttggggggatccccaggctgtggagcggccagagcaaggccagagcccggcaagggggtcgtggagaaaagcctggggctggctgctgctgacctgaacagtggtagcgacagccatccccaagccaaagctacagggagcgcaggcaccgagatagctcctgcggggtctgcaactgcggctccctgtgccccgcatcccgctcccagatccaggcaccacctcaagggctcgagggaagggaaagaaggagaacagatctggtttcccaaatgctggattccctcccctaaaaagcagccgccccgccatagccagacactccccagaccctgggctcccggaggcaccggatggagagaatctctgggtcttggagagggggcaggaccggagaccctggagggttggaaggcgacccgccgtgcccacaccttgccccgcagttcccagggcctgtcccgtggggaaggcgtctttgtcattgacgccacgtgcgtagtgatacgatcccaatatgttccaaccccccgaacccagcaggtgcagcttttgccctctggggtgacacgcgtggtgggggattcccccagccaatcgaagcccggcaaggaggagggtgaaggggccacggtctttccttccccttgtcaaaagcggctgtcgagcagtcgccttttacaccagcccggcgggggccgcgggggcgaagctgagggcgggaggccgggggactccacactggaggagcgcactttccgcatcttggggctcccggcccccgaagtaaacctgcgggacgcccccacgcagccaggtagcccagagcaccaagccttaggcccagcagcttcgggagcccagggcagagccgaggggtcggaagtggcggtggtccagcggcgcgccggccggggctgggcgcggaccccagggccctacgccggggccctgcgagaagccgtgtcccgtatccgccgccacacagcccctgactcggacacggacgaagctgaggagctcagcgtccatagcggctcctctgatggaagcgacacagaagccccgggcgcctcctggcggaatgagaggaccctgcccgaggttggaaacagttcgccagaggaagatgggaagacagcggaactgagcgacagtgtcggggagatcctagatgtcataagccaaaccgaggaggtcctcttcggggtgagggacatcagagggacccaacagggaaataggaagaggcagtgagaggccccttcttgtatttgtgtccccaacgcatccatccttgggtccactggtccccattcttccccacagacttcctttgcttctcttttccttgtatctttacccatacctgttctcatccttgaaatataaatgaaaggaagggaagcatatgcccattaatgattttgtttcaggagaggtgagaatgagcagatttaattaatgtctgttatgttcagggcacaagggtgagctcttcgcaggggctgatgcactgggtgtggagctgagcagagaggcctaaccaggatcaggcaggagggcagggatggtggcagccataggagggcagggtagggtagggcctctgaggaggagggaaaaagtgaaggagaggctttggacctggtgacagagtgatcagatgacagaggggttcttgggagaagaggcataggtccagcaacaaccaacaaagcagaaggagggctcaccttggtgtcacaagtcttggatttcaatcccaactctgccactgagttgctggttgactgaggccagtcactttccctctccaggcctccaggcctcctggtatataaaatgatggtattctaaggtccatccttccgtctctgacattttgagatctttggaaaggactctatctcatcctcccctcgacaagccaagaatgagaattgggaataagtgaacagagtttgagggtttctgggcggcctccgtgtcacccaaagtcatgatcaattcaggagactgcccaaggcttgcagaagaggtaagggagtgaggcactcctatcccagtctcccaggtttggttgagggctccccaaggcagggcaagatagcggccctgtcactgaccctggcctgtggtggtctgagctggggagggaaggacaccaatgaatcagcttgggacctctttaggccttccccttttcctccaccccgatgctccttagtgatgctctgaggcgtggccacgatctccctcccaggtggtatcgcccacctgaaaaaatcctgagaatttctcccatcttggcctcttccagaaaccggccaggcaaggaaagaggccggtcaccagaagccagcaggcgtggggtgtgatactctctatagccactacagggcgcgcgcaggtcgcggatctccccagttgctaatcccggctctgccactcaatcctatccctagttcccgagcgcgggtcccccgccttgcagtctccagccgtgcggggccgggagcaggcctccggcctcccagacttctagagcccgccgggcccatctttgtactcatccaccccagccggcttgggactcagacaccgaagtctttttttttttctctccgatccttggacacctcctctgtctgccatttattagccatgtgaacttggccacatcacttcacctccctgagcctcagtttcctcatctgtcaaatgggggtttataaacacctacctcgcagggttgttgtgaggatttaatgcgataatgtatgtaaagcgccttgcacactgcctggcacacagtaggcgctcaataaatctaagcttcccttta

SEQ ID NO: 28 - protein, 710 aa, Homo sapiens
MLDGPLFSEGPDSPRELQDEESGSCLWVQKSKLLVIEVKTISCHYSRRAPSRQPMDFQASHWARGFQNRTCGPRPGSPQPPPRRPWASRVLQEATNWRAGPLAEVRAREQEKRKAASQEREAKETERKRRKAGGARRSPPGRPRPEPRNAPRVAQLAGLPAPLRPERLAPVGRAPRPSAQPQSDPGSAWAGPWGGRRPGPPSYEAHLLLRGSAGTAPRRRWDRPPPYVAPPSYEGPHRTLGTKRGPGNSQVPTSSAPAATPARTDGGRTKKRLDPRIYRDVLGAWGLRQGQGLLGGSPGCGAARARPEPGKGVVEKSLGLAAADLNSGSDSHPQAKATGSAGTEIAPAGSATAAPCAPHPAPRSRHHLKGSREGKEGEQIWFPKCWIPSPKKQPPRHSQTLPRPWAPGGTGWRESLGLGEGAGPETLEGWKATRRAHTLPRSSQGLSRGEGVFVIDATCVVIRSQYVPTPRTQQVQLLPSGVTRVVGDSPSQSKPGKEEGEGATVFPSPCQKRLSSSRLLHQPGGGRGGEAEGGRPGDSTLEERTFRILGLPAPEVNLRDAPTQPGSPEHQALGPAASGAQGRAEGSEVAVVQRRAGRGWARTPGPYAGALREAVSRIRRHTAPDSDTDEAEELSVHSGSSDGSDTEAPGASWRNERTLPEVGNSSPEEDGKTAELSDSVGEILDVISQTEEVLFGVRDIRGTQQGNRKR

SEQ ID NO: 29 - DNA, 2694 bp, Homo sapiens
attataatgcaagaagccccctttttaaccacaaaccgaattttctttcatttaggtgatctatatatatctatatcgtatagcttatagcttatatctattttaaataacttaaagccgctaaaatttgggggggaacagctttcgccctggagcggtgcgcgatgctgtctcatgccgacctcctggacgccaggctaggtatgaaagatgccgccgagcttctgggccaccgggaggcggtgaagtgtaggctgggcgtggggggctccgaccccgggggccatccgggggacctggcgcccaactctgacccagtggagggagccactctgctgcccggggaggacatcaccacagtgggctctactccggcctcgctggcggtgagcgccaaagacccggacaagcagcccgggccccagggcggcccgaaccccagccaagccggccagcagcagggccaacagaagcagaagcgccaccggacgcgcttcacccccgcacagctcaacgagttggagaggagcttcgccaagactcactaccccgacatctttatgcgtgaggagctggcactgcgtatcgggctgaccgagtcccgagtgcaggtctggttccagaaccgacgcgccaagtggaagaagcgcaaaaagacgaccaacgtgttccgtgcgcccggcacactgctgcccacgccaggcctgcctcagttcccgtcggctgccgccgccgctgccgccgccatgggcgacagcctgtgctctttccacgccaacgacacccgctgggcggcggccgccatgcctggcgtgtcacagctgcctctgccgccggcgctgggcaggcagcaggccatggcgcagtcgctgtcccagtgcagcctggcggccggtccgccgcccaactccatgggcctgtccaacagcctggcgggttccaacggcgcggggctgcagtcgcacctctaccagcccgccttccccggcatggtgcccgcctccctccccggccccagcaacgtctccggttcgccccagctctgcagctccccggacagcagcgacgtgtggcggggcaccagcatcgcctccctccgccgcaaggcgctagagcacacagtctctatgagcttcacttaatgcagccgcgccccggcccgctccgcccccagcaccgccccgggggccgccccgaggcccttccggcgcgcacccggaccccggcgccctgccccgtcccgccccggccttcgccccgtctcgtttcgtcctcgcctctctcctccactcgctcgggctcaccccaagccccagcccgcgaggcctcccctccgcctgatttcgatcgcccgcggtcccccgtctcccggccgcccctcttcccttcccacccagctgcgccctcggctcggtctccagcgcctcagcccacccttcccgccaccctggcctccctgcttgcgctggccgtgctcgcgccctcctcctggccttctgacgggcggcgttcccacccacaccttcgacgcgacgcctacgacccccctcgcccgccgcctcccctccggtcccctctttccccacacttcgcgaccctcctcccgcgcccggcaaaaagtatccttcccgccattttacgtaccagggagtcgactcaggatctgaaatcagacaccaatggactggtttgtgggcagaaacacacacactcgcactctcgctcacgctcagacgctacacacgcgcgcgcacagacacggtgcacctaggtcacacacggacgtgttcaagggacagcacaatgttagggatttttgtcttaaaggaggacaagcattgctaccaaccgcctcatctgagggcccaactgatatgatttgatttatccttgtactctccaagctcctgtctttctttcctctcccaccacgctacccttgcccagtccacccagtcacatccgtgcagccctctcttggcttgcaagataacgcttttatttttattttatcttattttcattttcttaagcacaactgtgtgagagtgtagaagggaaggcttctcaggaggaacgtgacagtggattgggtggctggagtagactaaagcagtcatgtgacgaggaagaggtgatctgacccattttgataagtctttataaggaagaataaaataaacgtgtaagcaaaattttcttttgtaaaagcaaaagccacatctcttttctggatccttcaggactggggtttgtttgcttccttttctgtttctgtcttctcgctgctctgtgcccttggttgttttgtggtggtcctgtcgtccctcgtgcccctcggccacctgctggcagccgatgggggcactcggacatctacaaccctgcaactttgtacagagaaacacaatcagctctttctgcatgtgctggtcaaatccaaacccagagaacagaagcgctttctaagaatgaacaaatatgtgaaataggatgttttgtgtagataaagcattcttgttacatactggtcaatttgtgatatgttttaacttaatgtctgtgtttatttatggaattcggttttcttaataaatgtttgagctaatataaagcatattatttgacttttccggacaagtttatatcaagttaaatgtaaatggataaaataaaatcattttcagtatgtga

SEQ ID NO: 30 - protein, 325 aa, Homo sapiens
MLSHADLLDARLGMKDAAELLGHREAVKCRLGVGGSDPGGHPGDLAPNSDPVEGATLLPGEDITTVGSTPASLAVSAKDPDKQPGPQGGPNPSQAGQQQGQQKQKRHRTRFTPAQLNELERSFAKTHYPDIFMREELALRIGLTESRVQVWFQNRRAKWKKRKKTTNVFRAPGTLLPTPGLPQFPSAAAAAAAAMGDSLCSFHANDTRWAAAAMPGVSQLPLPPALGRQQAMAQSLSQCSLAAGPPPNSMGLSNSLAGSNGAGLQSHLYQPAFPGMVPASLPGPSNVSGSPQLCSSPDSSDVWRGTSIASLRRKALEHTVSMSFT